(19) United States
(12) Patent Application Publication
(10) Pub. No.: US 2016/0013423 A1
(43) Pub. Date: Jan. 14, 2016

(54) CONDENSED CYCLIC COMPOUND AND ORGANIC LIGHT-EMITTING DEVICE INCLUDING THE SAME

(71) Applicants: Samsung Electronics Co., Ltd., Suwon-si (KR); Samsung SDI Co., Ltd., Yongin-si (KR)

(72) Inventors: Dalho HUH, Suwon-si (KR); Miyoung CHAE, Suwon-si (KR); Hyunjung KIM, Suwon-si (KR); Soonok JEON, Seoul (KR); Yeonsook CHUNG, Seoul (KR); Yongsik JUNG, Yongin-si (KR); Wook KIM, Suwon-si (KR); Jhunmo SON, Suwon-si (KR); Namheon LEE, Suwon-si (KR); Sangmo KIM, Hwaseong-si (KR)

(21) Appl. No.: 14/795,075

(22) Filed: Jul. 9, 2015

(30) Foreign Application Priority Data
Jul. 9, 2014 (KR) 10-2014-0086329
Jul. 8, 2015 (KR) 10-2015-0097108

(51) Int. Cl. H01L 51/00; C07D 401/14; C09K 11/02; C07D 409/14; C09K 11/06; C07D 405/14; C07D 209/88

(52) U.S. Cl. CPC H01L 51/0072; C07D 405/14; C07D 401/14; C07D 209/88; C07D 409/14; H01L 51/0085; C09K 11/06; C09K 11/02; H01L 51/0073; H01L 51/0067; H01L 51/0074; H01L 51/5016

(57) ABSTRACT

A condensed cyclic compound represented by Formula 1:

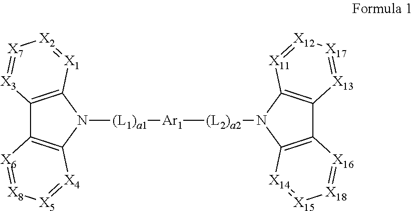

wherein in Formula 1, Ar₁, L₁, L₂, X₁ to X₁₆, a1, and a2 are the same as described in the specification.

### CROSS-REFERENCE TO RELATED APPLICATION

This application claims priority to and the benefit of Korean Patent Application Nos. 10-2014-0086329, filed on Jul. 9, 2014, and 10-2015-0097108, filed on Jul. 8, 2015, in the Korean Intellectual Property Office, the disclosure of which is incorporated herein in its entirety by reference.

### BACKGROUND

1. Field

The present disclosure relates to a condensed cyclic compound and an organic light-emitting device including the same.

2. Description of the Related Art

Organic light-emitting devices (OLEDs) are self-emission devices that have wide viewing angles, high contrast ratios, and short response times. In addition, OLEDs exhibit excellent brightness, driving voltage, and response speed characteristics, and produce full-color images, compared to electronic devices of the related art.

In an example, an organic light-emitting device includes an anode, a cathode, and an organic layer that is disposed between the anode and the cathode and includes an emission layer. A hole transport region may be disposed between the anode and the emission layer, and an electron transport region may be disposed between the emission layer and the cathode. Holes provided from the anode may move toward the emission layer through the hole transport region, and electrons provided from the cathode may move toward the emission layer through the electron transport region. Carriers, such as holes and electrons, are recombined in the emission layer to produce excitons. These excitons change from an excited state to a ground state, thereby generating light.

Various types of organic light emitting devices are known. However, there still remains a need in OLEDs having low driving voltage, high efficiency, high brightness, and long lifespan.

### SUMMARY

Provided are novel condensed cyclic compounds and organic light-emitting devices including the same.

Additional aspects will be set forth in part in the description which follows and, in part, will be apparent from the description, or may be learned by practice of the presented exemplary embodiments.

According to an aspect of an exemplary embodiment, a condensed cyclic compound is represented by Formula 1:

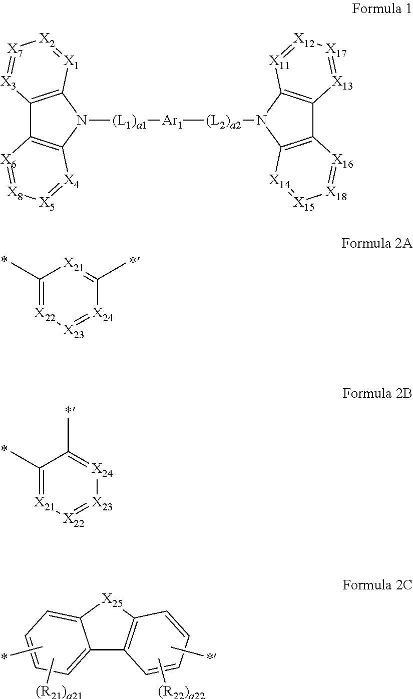

wherein in the formulae above,

X₁ may be N or C(R₁), X₂ may be N or C(R₂), X₃ may be N or C(R₃), X₄ may be N or C(R₄), X₅ may be N or C(R₅), X₆ may be N or C(R₆), X₇ may be N or C(R₇), X₈ may be N or C(R₃), X₁₁ may be N or C(R₁₁), X₁₂ may be N or C(R₁₂), X₁₃ may be N or C(R₁₃), X₁₄ may be N or C(R₁₄), X₁₅ may be N or C(R₁₅), X₁₆ may be N or C(R₁₆), X₁₇ may be N or C(R₁₇), X₁₈ may be N or C(R₁₈),

R₁ to R₈ and R₁₁ to R₁₈ may be each independently selected from a hydrogen, a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₇-C₆₀ arylalkyl group, a substituted or unsubstituted C₁-C₆₀ heteroaryl group, a substituted or unsubstituted C₂-C₆₀ heteroaryloxy group, a substituted or unsubstituted C₂-C₆₀ heteroarylthio group, a substituted or unsubstituted C₃-C₆₀ heteroarylalkyl group, a substituted or unsubstituted monovalent non-aromatic condensed polycyclic group, a substituted or unsubstituted monovalent non-aromatic condensed heteropolycyclic group (provided that the substituted or unsubstituted monovalent non-aromatic condensed heteropolycyclic group is not a substituted or unsubstituted carbazolyl group), and —Si(Q₁)(Q₂)(Q₃),

at least one selected from X₇, X₃, X₁₇ and X₁₈ is C(CN),

Ar₁ is represented by one of Formulae 2A to 2C,

X₂₁ may be N or C(R₂₁), X₂₂ may be N or C(R₂₂), X₂₃ may be N or C(R₂₃), X₂₄ may be N or C(R₂₄), X₂₅ may be O, S, P(═O)(R₂₅), Se, or Si(R₂₅)(R₂₆),

R₂₁ to R₂₆ may be each independently selected from a hydrogen, a deuterium, C₁-C₄ alkyl group, a phenyl group, a pyridinyl group, a pyrimidinyl group, a pyrazinyl group, a pyridazinyl group, a triazinyl group, and —Si(Q₁₁)(Q₁₂)(Q₁₃),

a21 and a22 may be each independently an integer selected from 0 to 3, a21 is 2 or greater, two or more groups R₂₁ may be identical to or different from each other, a22 is 2 or greater, two or more groups R₂₂ may be identical to or different from each other;

L₁ and L₂ may be each independently selected from

a phenylene group, a pyridinylene group, a pyrimidinylene group, a pyrazinylene group, a pyridazinylene group, a triazinylene group, a dibenzofuranylene group, and a dibenzothiophenylene group; and

a phenylene group, a pyridinylene group, a pyrimidinylene group, a pyrazinylene group, a pyridazinylene group, a triazinylene group, a dibenzofuranylene group, and a dibenzothiophenylene group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, a phenyl group, a naphthyl group, a pyridinyl group, a pyrimidinyl group, a pyrazinyl group, a pyridazinyl group, a triazinyl group and —Si(Q₂₁)(Q₂₂)(Q₂₃),

a1 and a2 may be each independently an integer selected from 0 to 5, and when a1 is 2 or greater, two or more groups L₁ may be identical to or different from each other, and when a2 is 2 or greater, two or more groups L₂ may be identical to or different from each other;

each of * and *′ indicates a binding site to a neighboring atom; and

at least one of the substituted C₁-C₆₀ alkyl group, substituted C₂-C₆₀ alkenyl group, substituted C₂-C₆₀ alkynyl group, substituted C₁-C₆₀ alkoxy group, substituted C₃-C₁₀ cycloalkyl group, substituted C₁-C₁₀ heterocycloalkyl group, substituted C₃-C₁₀ cycloalkenyl group, substituted C₁-C₁₀ heterocycloalkenyl group, substituted C₆-C₆₀ aryl group, substituted C₆-C₆₀ aryloxy group, substituted C₆-C₆₀ arylthio group, a substituted or unsubstituted C₇-C₆₀ arylalkyl group, substituted C₁-C₆₀ heteroaryl group, a substituted or unsubstituted C₂-C₆₀ heteroaryloxy group, a substituted or unsubstituted C₂-C₆₀ heteroarylthio group, a substituted or unsubstituted C₃-C₆₀ heteroarylalkyl group, substituted monovalent non-aromatic condensed polycyclic group, and substituted monovalent non-aromatic condensed heteropolycyclic group may be selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, a monovalent non-aromatic condensed heteropolycyclic group (provided that the monovalent non-aromatic condensed heteropolycyclic group is not a carbazolyl group), and —Si(Q₃₁)(Q₃₂)(Q₃₃),

wherein Q₁ to Q₃, Q₁₁ to Q₁₃, Q₂₁ to Q₂₃, and Q₃₁ to Q₃₃ may be each independently selected from a hydrogen, a C₁-C₆₀ alkyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group (provided that the monovalent non-aromatic condensed heteropolycyclic group is not a carbazolyl group), provided that the condensed cyclic compound is not the following compound:

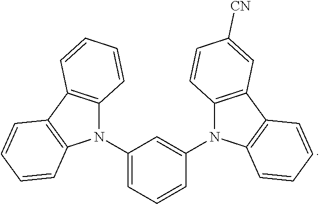 [SMILES: N#Cc1ccc2c(c1)c1ccccc1n2-c1cccc(-n2c3ccccc3c3ccccc32)c1]

According to another aspect of an exemplary embodiment, an organic light-emitting device includes:

a first electrode,

a second electrode, and

an organic layer disposed between the first electrode and the second electrode,

wherein the organic layer including an emission layer and at least one of the condensed cyclic compounds represented by Formula 1 described above.

### BRIEF DESCRIPTION OF THE DRAWINGS

These and/or other aspects will become apparent and more readily appreciated from the following description of the exemplary embodiments, taken in conjunction with the accompanying drawings in which:

FIG. 1 shows a schematic cross-sectional view of an organic light-emitting device according to an exemplary embodiment;

FIG. 2 shows heat-resistance evaluation results of a mCP-thin film, a Compound A-thin film, and a Compound 4-thin film;

FIG. 3 is a graph of efficiency (arbitrary units, a. u.) versus luminance (candelas per square meter, cd/m²) showing luminance-efficiency of organic light-emitting devices manufactured according to Examples 1 to 3 and Comparative Examples 1 and 2;

FIG. 4 is a graph of efficiency (arbitrary units, a. u.) versus luminance (candelas per square meter, cd/m²) showing a voltage-current density graph of organic light-emitting devices manufactured according to Examples 1 to 3 and Comparative Examples 1 and 2; and

FIG. 5 is a graph of luminance (percent, %) versus time (hours, hr) showing time-luminance of organic light-emitting devices manufactured according to Examples 1 to 3 and Comparative Examples 1 and 2.

### DETAILED DESCRIPTION

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the figures, to explain aspects. As used herein, the term “and/or includes any and all combinations of one or more of the associated listed items. Expressions such as at least one of,” when preceding a list of elements, modify the entire list of elements and do not modify the individual elements of the list.

It will be understood that when an element is referred to as being “on” another element, it can be directly in contact with the other element or intervening elements may be present therebetween. In contrast, when an element is referred to as being “directly on” another element, there are no intervening elements present.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

The terminology used herein is for the purpose of describing particular embodiments only and is not intended to be limiting. As used herein, the singular forms “a,” “an,” and “the” are intended to include the plural forms as well, unless the context clearly indicates otherwise.

It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Spatially relative terms, such as “beneath,” “below,” “lower,” “above,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10%, 5% of the stated value.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

A condensed cyclic compound according to an embodiment is represented by Formula 1:

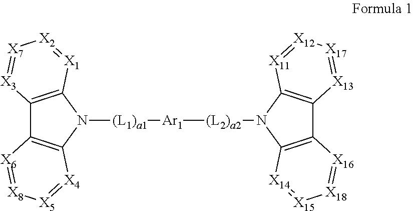

X₁ in Formula 1 may be N or C(R₁), X₂ may be N or C(R₂), X₃ may be N or C(R₃), X₄ may be N or C(R₄), X₅ may be N or C(R₅), X₆ may be N or C(R₆), X₇ may be N or C(R₇), X₈ may be N or C(R₅), X₁₁ may be N or C(R₁₁), X₁₂ may be N or C(R₁₂), X₁₃ may be N or C(R₁₃), X₁₄ may be N or C(R₁₄), X₁₅ may be N or C(R₁₅), X₁₆ may be N or C(R₁₆), X₁₇ may be N or C(R₁₇), and X₁₈ may be N or C(R₁₈).

In some embodiments, each of X₁ to X₈ and X₁₁ to X₁₈ in Formula 1 may not be N.

In some embodiments, one selected from X₁ to X₈ and X₁₁ to X₁₈ in Formula 1 may be N, and the others may not be N.

In some embodiments, X₁ may be N, and each of X₂ to X₈ and X₁₁ to X₁₈ may not be N, but they are not limited thereto.

In some embodiments, X₇ may be N, and each of X₁ to X₆, X₈ and X₁₁ to X₁₈ may not be N, but they are not limited thereto.

R₁ to R₈ and R₁₁ to R₁₈ may be each independently selected from a hydrogen, a deuterium, —F, —Cl, —Br, —I, a hydroxyl group, a cyano group (CN), a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₇-C₆₀ arylalkyl group, a substituted or unsubstituted C₁-C₆₀ heteroaryl group, a substituted or unsubstituted C₂-C₆₀ heteroaryloxy group, a substituted or unsubstituted C₂-C₆₀ heteroarylthio group, a substituted or unsubstituted C₃-C₆₀ heteroarylalkyl group, a substituted or unsubstituted monovalent non-aromatic condensed polycyclic group, a substituted or unsubstituted monovalent non-aromatic condensed heteropolycyclic group (provided that the substituted or unsubstituted monovalent non-aromatic condensed heteropolycyclic group is not a substituted or unsubstituted carbazolyl group), and —Si(Q₁)(Q₂)(Q₃).

For example, R₁ to R₈ and R₁₁ to R₁₆ may be each independently selected from

a hydrogen, a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, and a C₁-C₂₀ alkoxy group;

a C₁-C₂₀ alkyl group and a C₁-C₂₀ alkoxy group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a phenyl group, a naphthyl group, a pyridinyl group, a pyrimidinyl group, a pyrazinyl group, a pyridazinyl group, and a triazinyl group;

a cyclopentyl group, a cyclohexyl group, a cyclopentenyl group, a cyclohexenyl group, a cycloheptenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a phenalenyl group, a phenanthrenyl group, an anthracenyl group, a fluoranthrenyl group, a triphenylenyl group, a pyrenyl group, a chrysenyl group, a naphthacenyl group, a picenyl group, a perylenyl group, a pentaphenyl group, a hexacenyl group, a pyrrolyl group, an imidazolyl group, a pyrazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzoxazolyl group, a benzoimidazolyl group, a furanyl group, a benzofuranyl group, a thiophenyl group, a benzothiophenyl group, a thiazolyl group, an isothiazolyl group, a benzothiazolyl group, an isoxazolyl group, an oxazolyl group, a triazolyl group, a tetrazolyl group, an oxadiazolyl group, a triazinyl group, a dibenzofuranyl group, a dibenzothiophenyl group, an imidazopyrimidinyl group and an imidazopyridinyl group;

a cyclopentyl group, a cyclohexyl group, a cyclopentenyl group, a cyclohexenyl group, a cycloheptenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a phenalenyl group, a phenanthrenyl group, an anthracenyl group, a fluoranthrenyl group, a triphenylenyl group, a pyrenyl group, a chrysenyl group, a naphthacenyl group, a picenyl group, a perylenyl group, a pentaphenyl group, a hexacenyl group, a pyrrolyl group, an imidazolyl group, a pyrazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzoxazolyl group, a benzoimidazolyl group, a furanyl group, a benzofuranyl group, a thiophenyl group, a benzothiophenyl group, a thiazolyl group, an isothiazolyl group, a benzothiazolyl group, an isoxazolyl group, an oxazolyl group, a triazolyl group, a tetrazolyl group, an oxadiazolyl group, a triazinyl group, a dibenzofuranyl group, a dibenzothiophenyl group, an imidazopyrimidinyl group, and an imidazopyridinyl group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₂-C₂₀ alkenyl group, a C₂-C₂₀ alkynyl group, a C₁-C₂₀ alkoxy group, a phenyl group, a naphthyl group, an anthracenyl group, a pyrenyl group, a phenanthrenyl group, a fluorenyl group, a pyridinyl group, a pyrimidinyl group, a pyrazinyl group, a pyridazinyl group, a triazinyl group, a quinolinyl group, an isoquinolinyl group, a phthalazinyl group, a quinoxalinyl group, a cinnolinyl group, a quinazolinyl group, and —Si(Q₃₁)(Q₃₂)(Q₃₃); and —Si(Q₁)(Q₂)(Q₃), wherein Q₁ to Q₃ and Q₃₁ to Q₃₃ may be each independently selected from a hydrogen, a C₁-C₂₀ alkyl group, a C₁-C₂₀ alkoxy group, a phenyl group, a naphthyl group, an anthracenyl group, a pyrenyl group, a phenanthrenyl group, a fluorenyl group, a pyridinyl group, a pyrimidinyl group, a pyrazinyl group, a pyridazinyl group, a triazinyl group, a quinolinyl group, an isoquinolinyl group a phthalazinyl group, a quinoxalinyl group, a cinnolinyl group, and a quinazolinyl group.

In some embodiments, R₁ to R₆ and R₁₁ to R₁₆ may be each independently selected from

a hydrogen, a deuterium, —F, —Cl, —Br, —I, a hydroxyl group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, and a C₁-C₁₀ alkoxy group;

a phenyl group, a naphthyl group, a pyridinyl group, a pyrimidinyl group, a pyrazinyl group, a pyridazinyl group, and a triazinyl group;

a phenyl group, a naphthyl group, a pyridinyl group, a pyrimidinyl group, a pyrazinyl group, a pyridazinyl group, and a triazinyl group, each substituted with at least one selected from a deuterium, —F, —Cl, —Br, —I, a hydroxyl group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, a phenyl group, a naphthyl group, a pyridinyl group, a pyrimidinyl group, a pyrazinyl group, a pyridazinyl group, a triazinyl group, and —Si(Q₃₁)(Q₃₂)(Q₃₃), and

—Si(Q₁)(Q₂)(Q₃),

wherein R₇, R₈, R₁₇, and R₁₈ may be each independently selected from

a hydrogen, a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, and a C₁-C₁₀ alkoxy group;

a phenyl group, a naphthyl group, a pyridinyl group, a pyrimidinyl group, a pyrazinyl group, a pyridazinyl group, and a triazinyl group;

a phenyl group, a naphthyl group, a pyridinyl group, a pyrimidinyl group, a pyrazinyl group, a pyridazinyl group, and a triazinyl group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, a phenyl group, a naphthyl group, a pyridinyl group, a pyrimidinyl group, a pyrazinyl group, a pyridazinyl group, a triazinyl group, and —Si(Q₃₁)(Q₃₂)(Q₃₃); and

—Si(Q₁)(Q₂)(Q₃),

wherein Q₁ to Q₃ and O₃₁ to Q₃₃ may be each independently selected from a hydrogen, a C₁-C₁₀ alkyl group, a C₁-C₁₀ alkoxy group, a phenyl group, a naphthyl group, a pyridinyl group, a pyrimidinyl group, a pyrazinyl group, a pyridazinyl group, and a triazinyl group, but are not limited thereto.

In some embodiments, R₁ to R₈ and R₁₁ to R₁₈ may be each independently selected from

a hydrogen, a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a methyl group, an ethyl group, an n-propyl group, an isopropyl group, an n-butyl group, an isobutyl group, a sec-butyl group, a tert-butyl group, an n-pentyl group, an isopentyl group, a sec-pentyl group, a tert-pentyl group, an n-hexyl group, an iso-hexyl group, a sec-hexyl group, a tert-hexyl group, an n-heptyl group, an iso-heptyl group, a sec-heptyl group, a tert-heptyl group, an n-octyl group, an iso-octyl group, a sec-octyl group, a tert-octyl group, an n-nonyl group, an iso-nonyl group, a sec-nonyl group, a tert-nonyl group, an n-decyl group, an isodecyl group, a sec-decyl group, a tert-decyl group, a methoxy group, an ethoxy group, a propoxy group, a butoxy group, a pentoxy group, a phenyl group, a naphthyl group, a pyridinyl group, a pyrimidinyl group, and a triazinyl group;

a methyl group, an ethyl group, an n-propyl group, an isopropyl group, an n-butyl group, an isobutyl group, a sec-butyl group, a tert-butyl group, an n-pentyl group, an isopentyl group, a sec-pentyl group, a tert-pentyl group, an n-hexyl group, an iso-hexyl group, a sec-hexyl group, a tert-hexyl group, an n-heptyl group, an iso-heptyl group, a sec-heptyl group, a tert-heptyl group, an n-octyl group, an iso-octyl group, a sec-octyl group, a tert-octyl group, an n-nonyl group, an iso-nonyl group, a sec-nonyl group, a tert-nonyl group, an n-decyl group, an isodecyl group, a sec-decyl group, a tert-decyl group, a methoxy group, an ethoxy group, a propoxy group, a butoxy group, a pentoxy group, a phenyl group, a naphthyl group, a pyridinyl group, a pyrimidinyl group, and a triazinyl group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof and a phosphoric acid group or a salt thereof; and

—Si(Q₁)(Q₂)(Q₃),

wherein Q₁ to Q₃ may be each independently selected from a hydrogen, a C₁-C₁₀ alkyl group, a C₁-C₁₀ alkoxy group and a phenyl group.

In some embodiments, R₁ to R₈ and R₁₁ to R₁₈ may be each independently selected from a hydrogen, a deuterium, a cyano group, a methyl group, an ethyl group, an n-propyl group, an isopropyl group, an n-butyl group, an isobutyl group, a sec-butyl group, a tert-butyl group, an n-pentyl group, an isopentyl group, a sec-pentyl group, a tert-pentyl group, an n-hexyl group, an iso-hexyl group, a sec-hexyl group, a tert-hexyl group, and —Si(Q₁)(Q₂)(Q₃),

wherein Q₁ to Q₃ may be each independently a hydrogen, a C₁-C₁₀ alkyl group, a C₁-C₁₀ alkoxy group, and a phenyl group.

At least one selected from X₇, X₈, X₁₇, and X₁₈ in Formula 1 may be C(CN).

For example, at least two selected from X₇, X₈, X₁₇ and X₁₈ in Formula 1 are each C(CN).

In some embodiments, X₁ may be C(R₁), X₂ may be C(R₂), X₃ may be C(R₃), X₄ may be C(R₄), X₅ may be C(R₅), X₆ may be C(R₆), X₇ may be C(R₇), X₈ may be C(R₅), X₁₁ may be C(R₁₁), X₁₂ may be C(R₁₂), X₁₃ may be C(R₁₃), X₁₄ may be C(R₁₄), X₁₅ may be C(R₁₅), X₁₆ may be C(R₁₆), X₁₇ may be C(R₁₇), X₁₈ may be C(R₁₈), and at least one selected from X₇, X₈, X₁₇, and X₁₈ may be C(CN). However, these are not limited thereto.

In some embodiments, X₁ may be N, X₂ may be C(R₂), X₃ may be C(R₃), X₄ may be C(R₄), X₅ may be C(R₅), X₆ may be C(R₆), X₇ may be C(R₇), X₈ may be C(R₃), X₁₁ may be C(R₁₁), X₁₂ may be C(R₁₂), X₁₃ may be C(R₁₃), X₁₄ may be C(R₁₄), X₁₅ may be C(R₁₅), X₁₆ may be C(R₁₆), X₁₇ may be C(R₁₇), X₁₈ may be C(R₁₈), and at least one selected from X₇, X₈, X₁₇, and X₁₈ may be C(CN). However, these are not limited thereto.

In some embodiments, X₁ may be C(R₁), X₂ may be C(R₂), X₃ may be C(R₃), X₄ may be C(R₄), X₅ may be C(R₅), X₆ may be C(R₆), X₇ may be N, X₈ may be C(R₈), X₁₁ may be C(R₁₁), X₁₂ may be C(R₁₂), X₁₃ may be C(R₁₃), X₁₄ may be C(R₁₄), X₁₅ may be C(R₁₅), X₁₆ may be C(R₁₆), X₁₇ may be C(R₁₇), X₁₈ may be C(R₁₈), and at least one selected from X₈, X₁₇, and X₁₈ may be C(CN). However, these are not limited thereto.

In an embodiment, R₁ to R₆ and R₁₁ to R₁₆ may each be not a cyano group.

Ar₁ in Formula 1 may be represented by one of Formulae 2A to 2C:

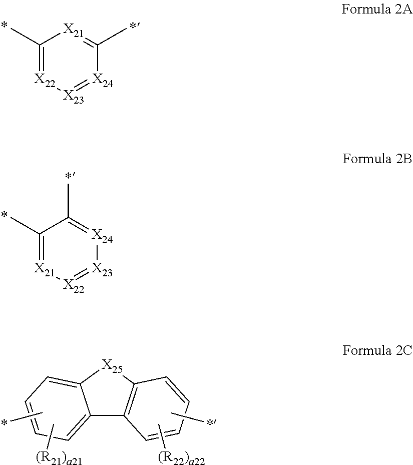

In Formulae 2A to 2C,

X₂₁ may be N or C(R₂₁), X₂₂ may be N or C(R₂₂), X₂₃ may be N or C(R₂₃), X₂₄ may be N or C(R₂₄), and X₂₅ may be O, S, P(═O)(R₂₅), Se, or Si(R₂₅)(R₂₆),

R₂₁ to R₂₆ may be each independently selected from a hydrogen, a deuterium, C₁-C₄ alkyl group, a phenyl group, a pyridinyl group, a pyrimidinyl group, a pyrazinyl group, a pyridazinyl group, a triazinyl group, and —Si(Q₁₁)(Q₁₂)(Q₁₃),

a21 and a22 may be each independently an integer selected from 0 to 3.

a21 indicates the number of groups R₂₁, and when a21 is 2 or greater, two or more groups R₂₁ may be identical to or different from each other. a2 indicates the number of groups R₂₂, and when a22 is 2 or greater, two or more R₂₂ may be identical to or different from each other.

In an embodiment, a1 and a2 may be each independently 0, 1, or 2.

In some embodiments, Ar₁ in Formula 1 may be represented by one of Formulae 2B and 2C.

For example, Ar₁ in Formula 1 may be represented by one of Formulae 2A-1 to 2A-10, 2B-1 to 2B-8, and 2C-1 to 2C-9:

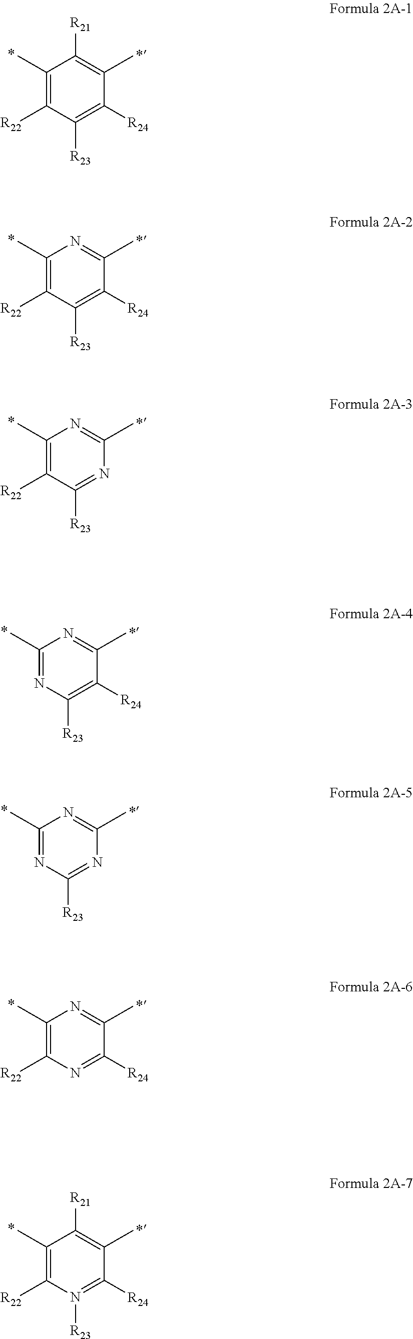

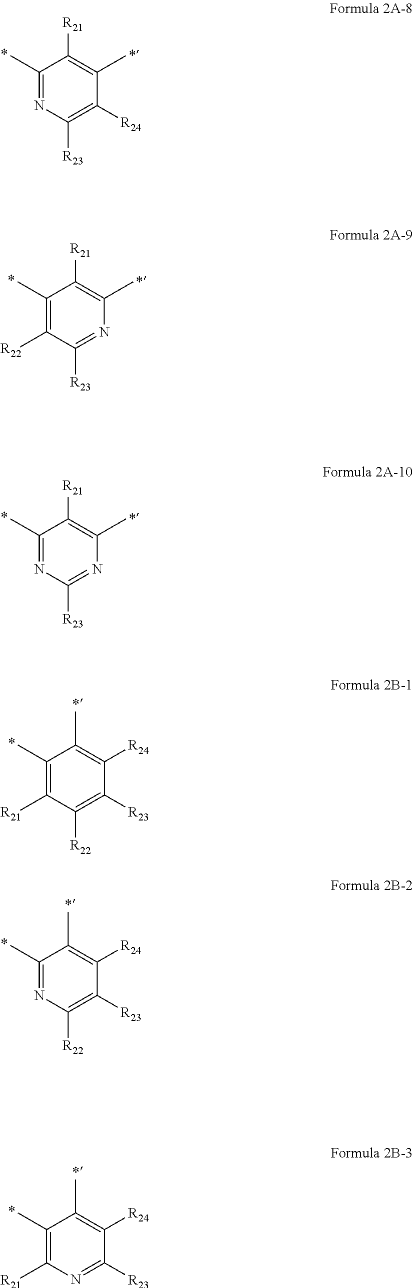

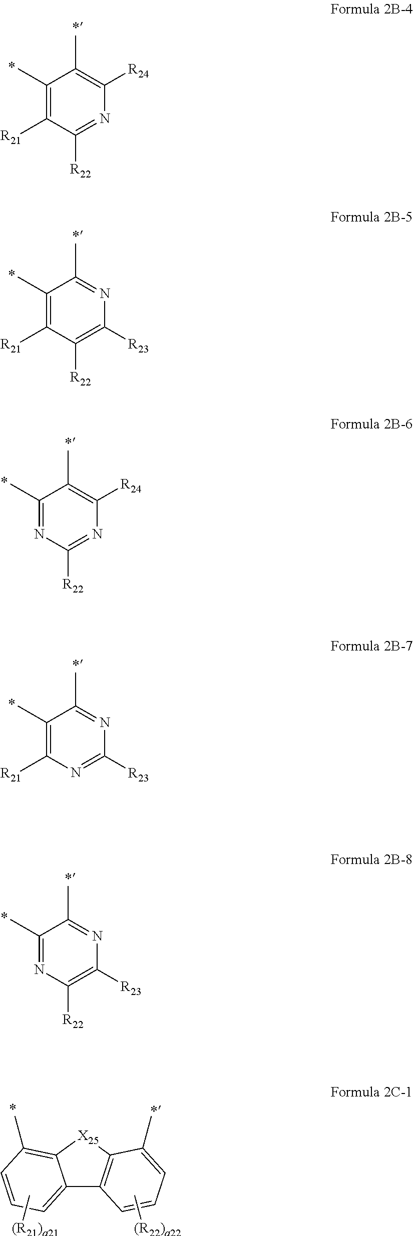

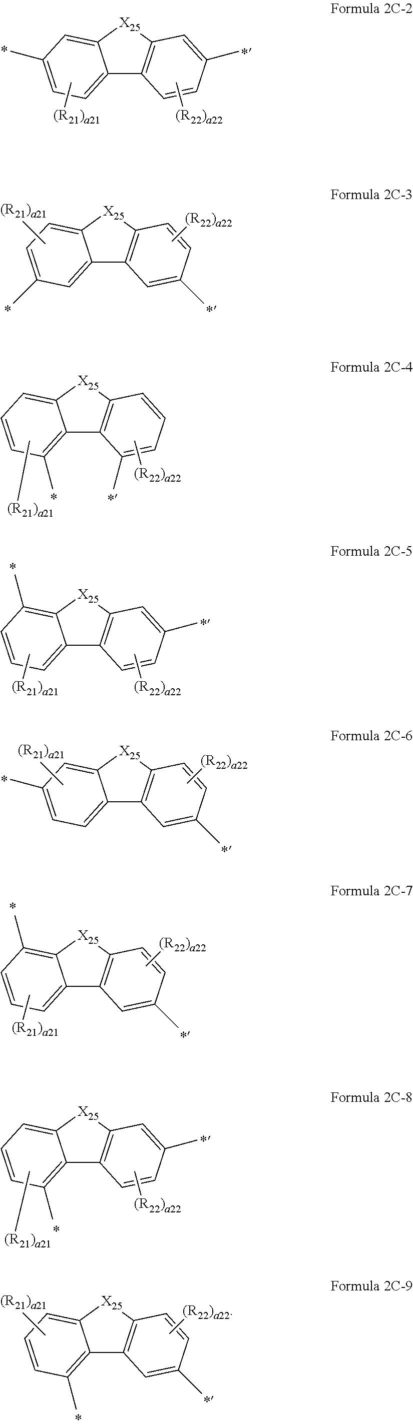

In Formulae 2A-1 to 2A-10, 2B-1 to 2B-8, and 2C-1 to 2C-9,

X₂₅ may be O, S, P(═O)(R₂₅), Se, or Si(R₂₅)(R₂₆),

R₂₁ to R₂₄ may be each independently selected from a hydrogen, a deuterium, a methyl group, an ethyl group, an n-propyl group, an isopropyl group, an n-butyl group, an isobutyl group, a sec-butyl group, a tert-butyl group, a phenyl group, a pyridinyl group, a pyrimidinyl group, a pyrazinyl group, a pyridazinyl group, a triazinyl group, and —Si(Q₁₁)(Q₁₂)(Q₁₃),

R₂₅ and R₂₆ may be each independently selected from a hydrogen, a deuterium, a methyl group, an ethyl group, an n-propyl group, an isopropyl group, an n-butyl group, an isobutyl group, a sec-butyl group, a tert-butyl group, a phenyl group, a pyridinyl group, a pyrimidinyl group, a pyrazinyl group, a pyridazinyl group, and a triazinyl group,

Q₁₁ to Q₁₃ may be each independently selected from a hydrogen, a C₁-C₁₀ alkyl group, a C₁-C₁₀ alkoxy group, and a phenyl group,

a21 and a22 may be each independently 0 or 1, and

each of * and *′ indicates a binding site to a neighboring atom.

In an embodiment, when Ar₁ in Formula 1 is represented by Formula 2A-1, the sum of a1 and a2 in Formula 1 may not be 0.

For example,

i) in Formula 1, one selected from X₇, X₈, X₁₇, and X₁₈ may be C(CN), the remainder may not be C(CN), and Ar₁ may be represented by one of Formulae 2A-2 to 2A-10, 2B-1 to 2B-8, and 2C-1 to 2C-9;

ii) in Formula 1, one selected from X₇, X₈, X₁₇, and X₁₈ may be C(CN), the remainder may not be C(CN), and the sum of a1 and a2 may not be 0; or

iii) in Formula 1, at least two selected from X₇, X₈, X₁₇ and X₁₈ may be each C(CN), are not limited thereto.

L₁ and L₂ in Formula 1 may be each independently selected from

a phenylene group, a pyridinylene group, a pyrimidinylene group, a pyrazinylene group, a pyridazinylene group, a triazinylene group, a dibenzofuranylene group, and a dibenzothiophenylene group; and

a phenylene group, a pyridinylene group, a pyrimidinylene group, a pyrazinylene group, a pyridazinylene group, a triazinylene group, a dibenzofuranylene group, and a dibenzothiophenylene group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, a phenyl group, a naphthyl group, a pyridinyl group, a pyrimidinyl group, a pyrazinyl group, a pyridazinyl group, a triazinyl group, and —Si(Q₂₁)(Q₂₂)(Q₂₃)

For example, L₁ and L₂ in Formula 1 may be each independently selected from

a phenylene group, a pyridinylene group, a pyrimidinylene group, and a triazinylene group; and

a phenylene group, a pyridinylene group, a pyrimidinylene group, and a triazinylene group, each substituted with at least one selected from a deuterium, a methyl group, an ethyl group, an n-propyl group, an isopropyl group, an n-butyl group, an isobutyl group, a sec-butyl group, a tert-butyl group, a phenyl group, a pyridinyl group, a pyrimidinyl group, a pyrazinyl group, a pyridazinyl group, a triazinyl group, and —Si(Q₂₁)(Q₂₂)(Q₂₃), but are not limited thereto.

a1 and a2 in Formula 1 may be each independently an integer selected from 0 to 5. a1 indicates the number of groups L₁, and when a1 is 0, *-(L₁)ₐ₁-*′ indicates a single bond. When a1 is 2 or greater, two or more groups L₁ may be identical to or different from each other. a2 indicates the number of groups L₂, when a2 is 0, *-(L₂)ₐ₂-*′ indicates a single bond. When a2 is 2 or greater, two or more groups L₂ may be identical to or different from each other.

In an embodiment, a1 and a2 may be each independently 0, 1, or 2.

In some embodiments, a1 and a2 may be each independently 0 or 1.

For example, L₁ and L₂ in Formula 1 may be each independently selected from

a phenylene group; and

a phenylene group, substituted with at least one selected from a deuterium, a methyl group, an ethyl group, an n-propyl group, an isopropyl group, an n-butyl group, an isobutyl group, a sec-butyl group, a tert-butyl group, a phenyl group, a pyridinyl group, a pyrimidinyl group, a pyrazinyl group, a pyridazinyl group, a triazinyl group, and —Si(Q₂₁)(Q₂₂)(Q₂₃),

wherein Q₂₁ to Q₂₃ may be each independently selected from a hydrogen, a C₁-C₁₀ alkyl group, a C₁-C₁₀ alkoxy group, and a phenyl group,

a1 and a2 may be each independently 0 or 1, but are not limited thereto.

In some embodiments, in Formula 1, a1 and a2 may each be 0, and Ar₁ may be represented by one of Formulae 2A-2 to 2A-10, 2B-1 to 2B-8, and 2C-1 to 2C-9.

In some embodiments, in Formula 1, a1 and a2 may each be 0, and Ar₁ may be represented by one of Formulae 2A-1, 2A-2, 2A-10, 2B-1, 2B-8, and 2C-3.

In some embodiments, the sum of a1 and a2 in Formula 1 is 1 or more, and *-(L₁)ₐ₁-Ar₁-(L₂)ₐ₂-*′ in Formula 1 may be represented by one of Formulae 3-1 to 3-57, but these are not limited thereto:

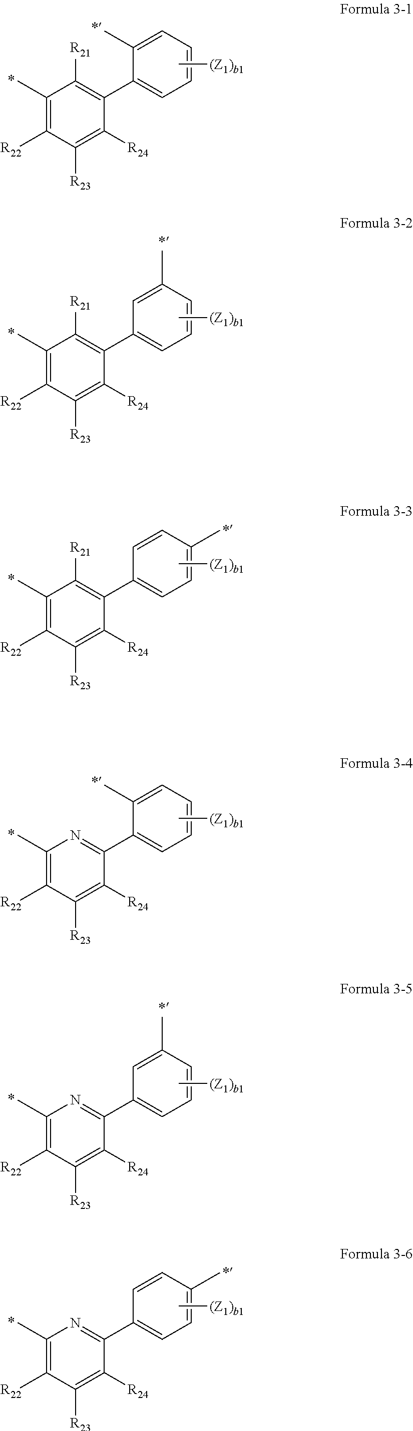

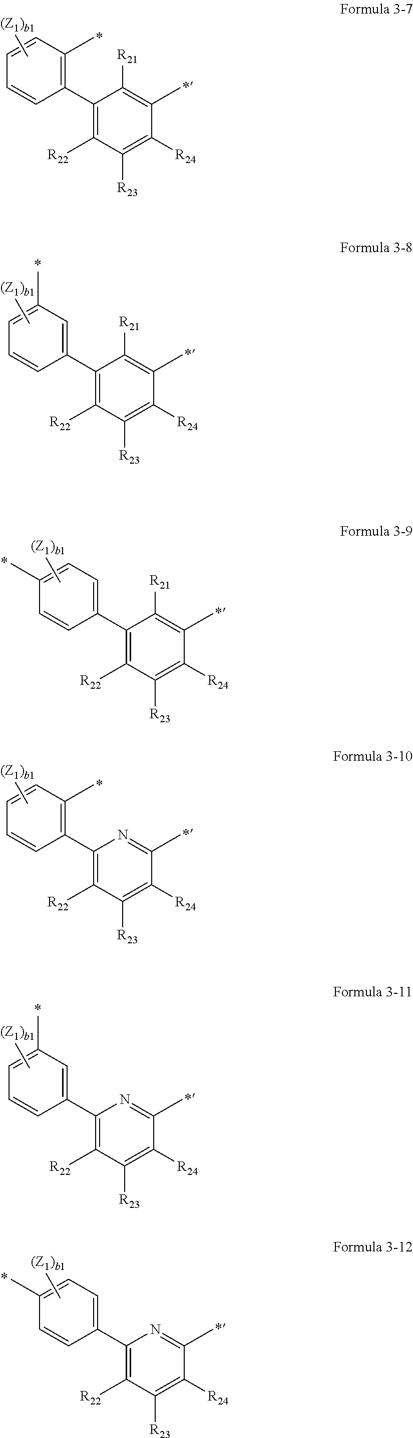

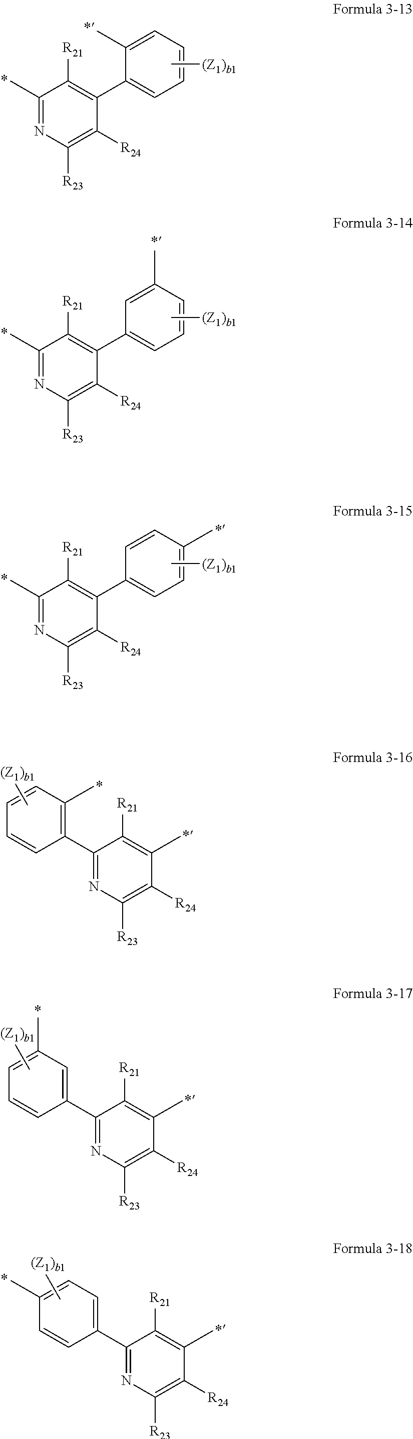

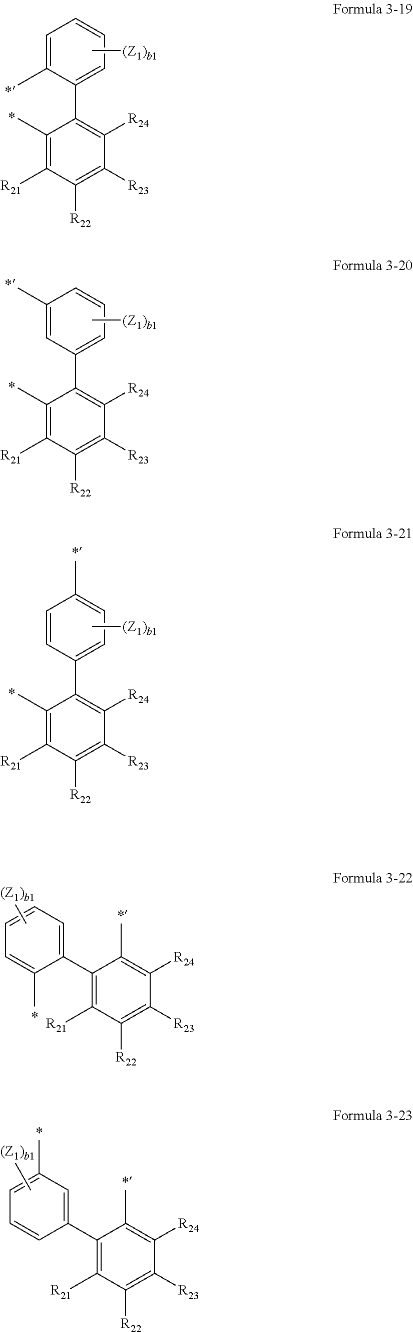

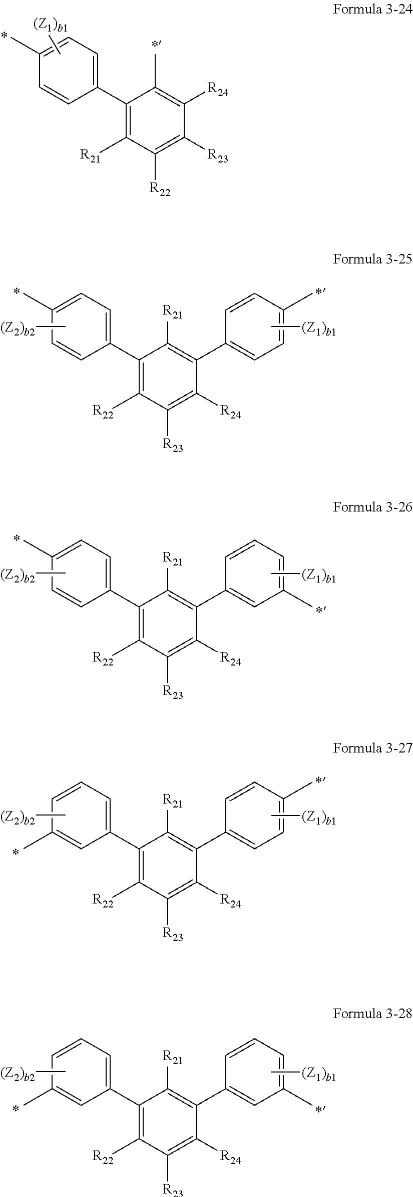

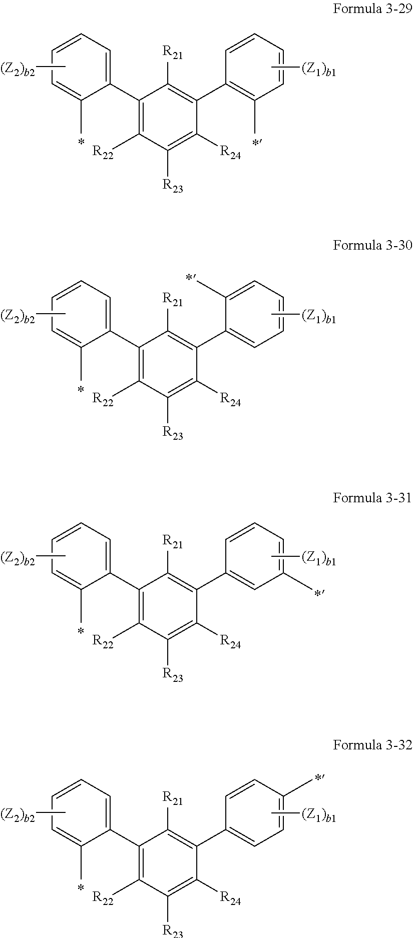

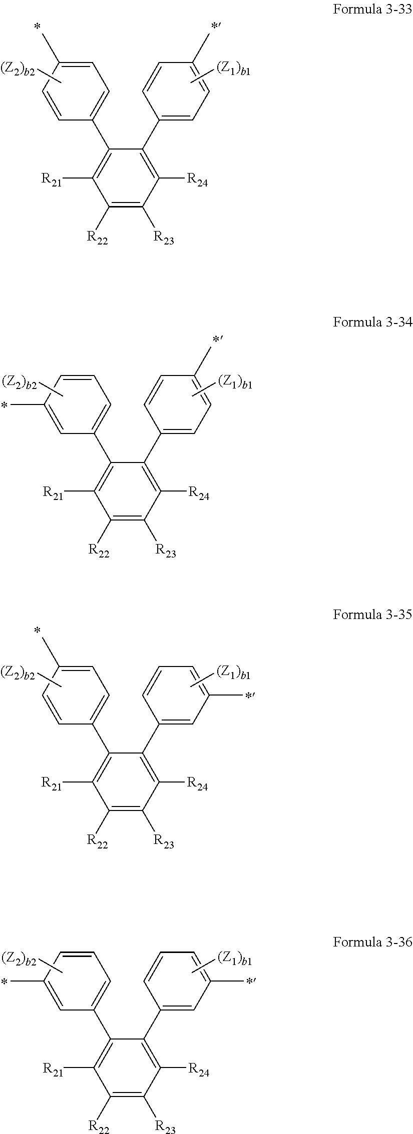

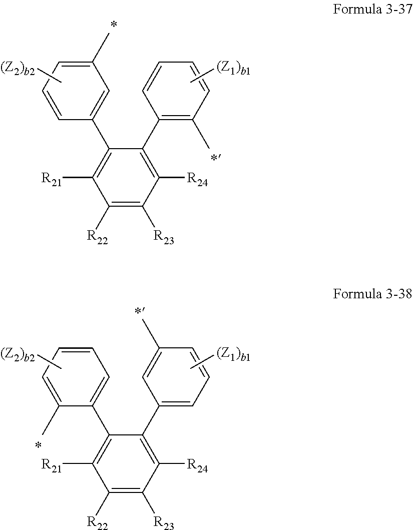

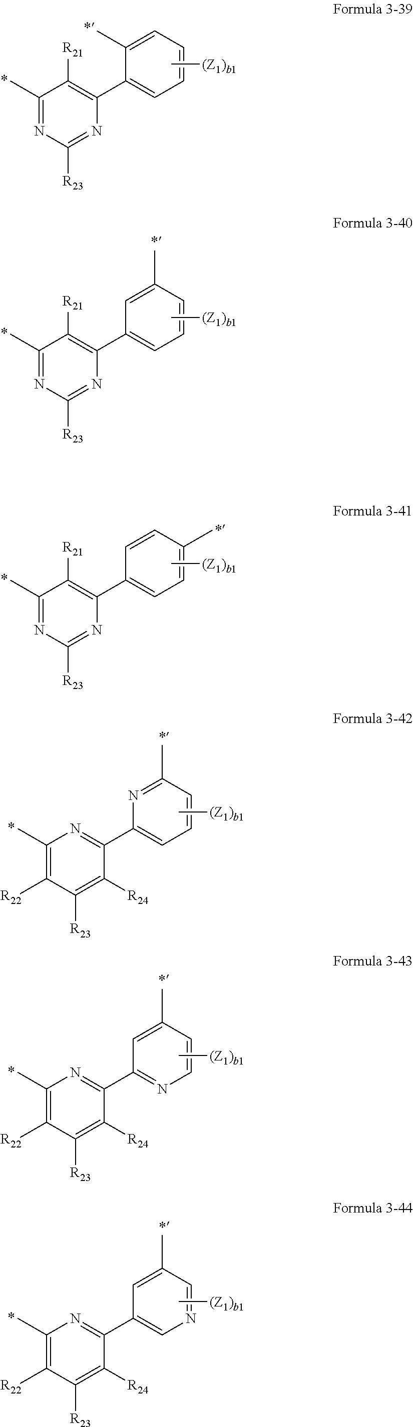

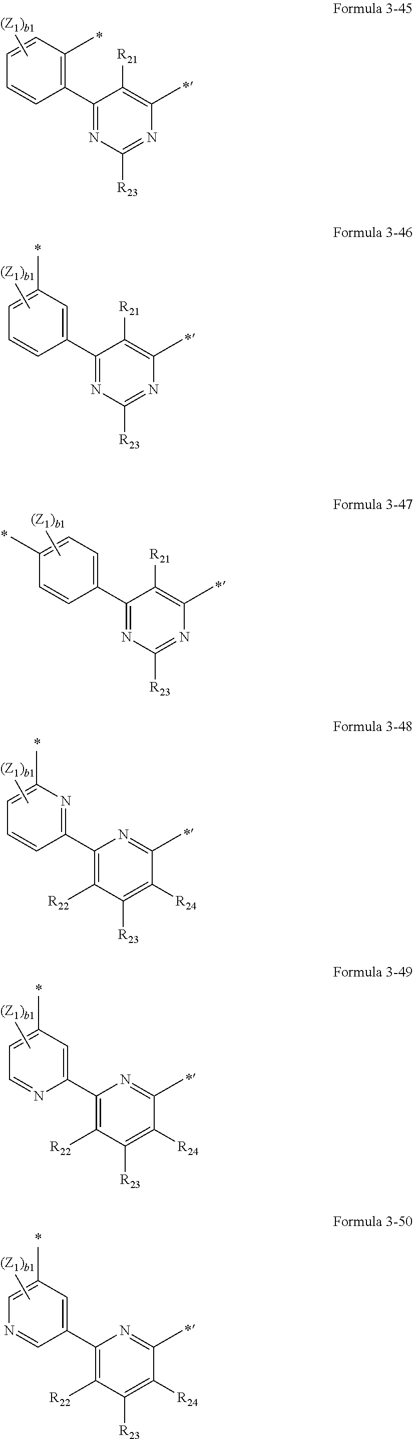

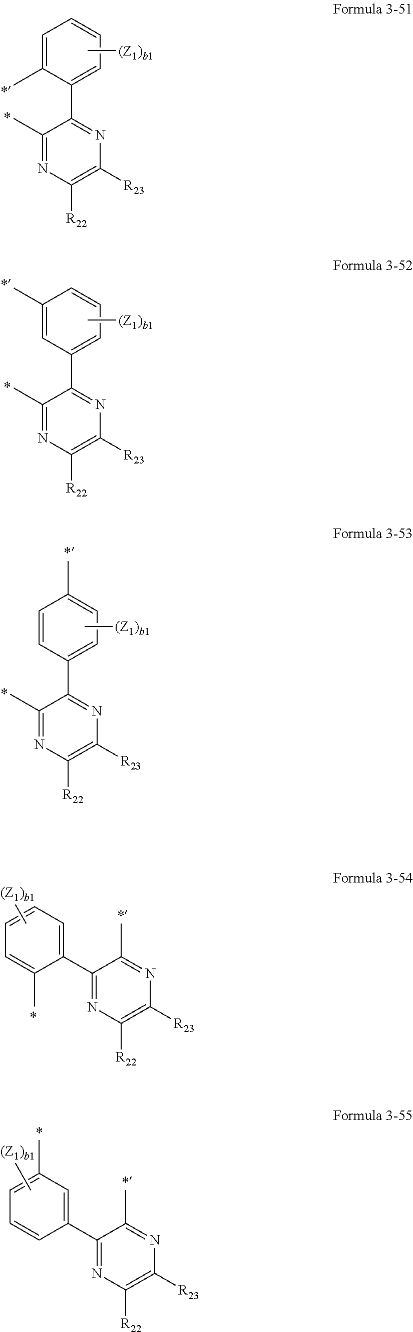

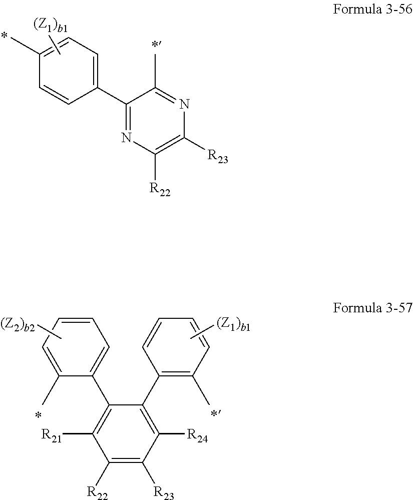

In Formulae 3-1 to 3-57,

R₂₁ to R₂₄, Z₁, and Z₂ may be each independently selected from a hydrogen, a deuterium, a methyl group, an ethyl group, an n-propyl group, an isopropyl group, an n-butyl group, an isobutyl group, a sec-butyl group, a tert-butyl group, a phenyl group, a pyridinyl group, a pyrimidinyl group, a pyrazinyl group, a pyridazinyl group, a triazinyl group, and —Si(Q₁₁)(Q₁₂)(Q₁₃),

Q₁₁ to Q₁₃ may be each independently selected from a hydrogen, a C₁-C₁₀ alkyl group, a C₁-C₁₀ alkoxy group, and a phenyl group,

b1 and b2 may be each independently 0, 1, or 2, and

each of * and *′ indicates a binding site to a nitrogen atom.

In an embodiment, the condensed cyclic compound represented by Formula 1 may be represented by one of Formulae 1A to 1E, but is not limited thereto:

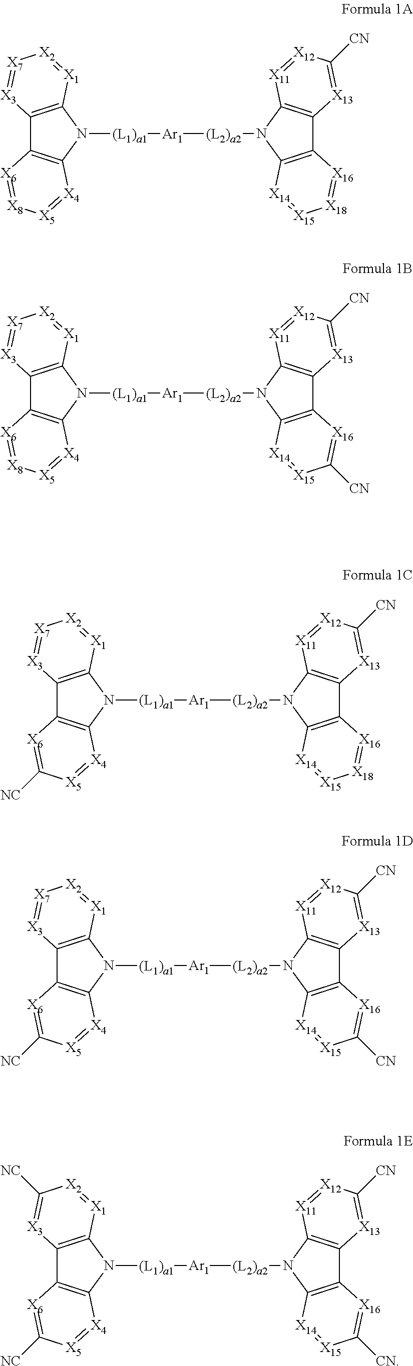

Descriptions of X₁ to X₈, X₁₁ to X₁₈, Ar₁, L₁, L₂, a1, a2, *-(L₁)ₐ₁-Ar₁-(L₂)ₐ₂-*′ in Formulae 1A to 1E are the same as presented above.

For example, in Formulae 1A to 1E, X₁ may be N or C(R₁), X₂ may be N or C(R₂), X₃ may be N or C(R₃), X₄ may be N or C(R₄), X₅ may be N or C(R₅), X₆ may be N or C(R₆), X₇ may be N or C(R₇), X₈ may be N or C(R₈), X₁₁ may be N or C(R₁₁), X₁₂ may be N or C(R₁₂), X₁₃ may be N or C(R₁₃), X₁₄ may be N or C(R₁₄), X₁₅ may be N or C(R₁₅), X₁₆ may be N or C(R₁₆), X₁₈ may be N or C(R₁₈), and R₁ to R₈, R₁₁ to R₁₆, and R₁₈ may not be a cyano group.

In some embodiments,

i) the condensed cyclic compound may be represented by Formula 1A in which Ar₁ is represented by one of Formulae 2A-2 to 2A-10, 2B-1 to 2B-8, and 2C-1 to 2C-9;

ii) the condensed cyclic compound may be represented by Formula 1A in which the sum of a1 and a2 is not 0; or

iii) the condensed cyclic compound may be represented by one of Formulae 1B to 1E.

In some embodiments, the condensed cyclic compound represented by Formula 1 may be represented by Formulae 1(1) to 1(3):

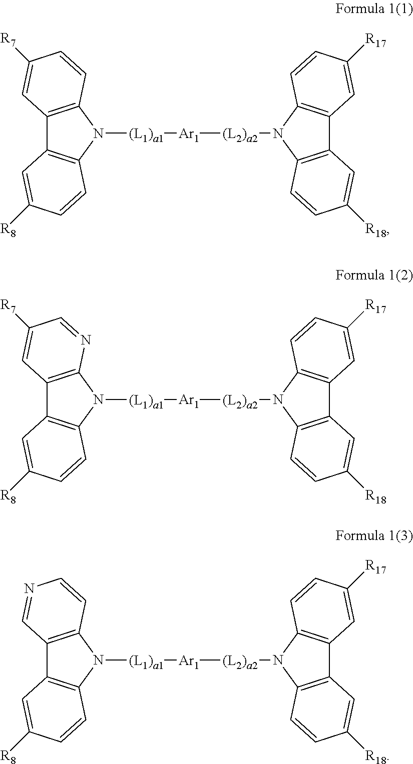

In Formulae 1(1) to 1(3),

descriptions of Ar₁, L₁, L₂, a1, a2, and *-(L₁)ₐ₁-Ar₁-(L₂)ₐ₂-*′ are the same as presented above,

R₇, R₈, R₁₇, and R₁₈ may be each independently selected from

a hydrogen, a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a methyl group, an ethyl group, an n-propyl group, an isopropyl group, an n-butyl group, an isobutyl group, a sec-butyl group, a tert-butyl group, an n-pentyl group, an isopentyl group, a sec-pentyl group, a tert-pentyl group, an n-hexyl group, an iso-hexyl group, a sec-hexyl group, a tert-hexyl group, an n-heptyl group, an iso-heptyl group, a sec-heptyl group, a tert-heptyl group, an n-octyl group, an iso-octyl group, a sec-octyl group, a tert-octyl group, an n-nonyl group, an iso-nonyl group, a sec-nonyl group, a tert-nonyl group, an n-decyl group, an isodecyl group, a sec-decyl group, a tert-decyl group, a methoxy group, an ethoxy group, a propoxy group, a butoxy group, a pentoxy group, a phenyl group, a naphthyl group, a pyridinyl group, a pyrimidinyl group, and a triazinyl group;

a methyl group, an ethyl group, an n-propyl group, an isopropyl group, an n-butyl group, an isobutyl group, a sec-butyl group, a tert-butyl group, an n-pentyl group, an isopentyl group, a sec-pentyl group, a tert-pentyl group, an n-hexyl group, an iso-hexyl group, a sec-hexyl group, a tert-hexyl group, an n-heptyl group, an iso-heptyl group, a sec-heptyl group, a tert-heptyl group, an n-octyl group, an iso-octyl group, a sec-octyl group, a tert-octyl group, an n-nonyl group, an iso-nonyl group, a sec-nonyl group, a tert-nonyl group, an n-decyl group, an isodecyl group, a sec-decyl group, a tert-decyl group, a methoxy group, an ethoxy group, a propoxy group, a butoxy group, a pentoxy group, a phenyl group, a naphthyl group, a pyridinyl group, a pyrimidinyl group and a triazinyl group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, and a phosphoric acid group or a salt thereof; and

—Si(Q₁)(Q₂)(Q₃),

wherein Q₁ to Q₃ may be each independently selected from a hydrogen, a C₁-C₁₀ alkyl group, a C₁-C₁₀ alkoxy group and a phenyl group,

at least one selected from R₇, R₈, R₁₇, and R₁₃ in Formulae 1(1) and 1(2) may be a cyano group (CN), and

at least one selected from R₃, R₁₇, and R₁₈ in Formula 1(3) may be a cyano group (CN).

In some embodiments, in Formulae 1(1) to 1(3),

i) Ar₁ may be represented by Formula 2A-1 and at least two selected from X₇, X₈, X₁₇ and X₁₈ may be each C(CN);

ii) Ar₁ may be represented by Formula 2A-1 and the sum of a1 and a2 may not be 0; or

iii) Ar₁ may be represented by one of Formulae 2A-2 to 2A-10, 2B-1 to 2B-8 and 2C-1 to 2C-9, but it may be seen that they are not limited thereto.

In some embodiments, the condensed cyclic compound represented by Formula 1 may be represented by one selected from Formulae 1A(1) to 1E(1), 1A(2) to 1E(2), and 1A(3) to 1 D(3):

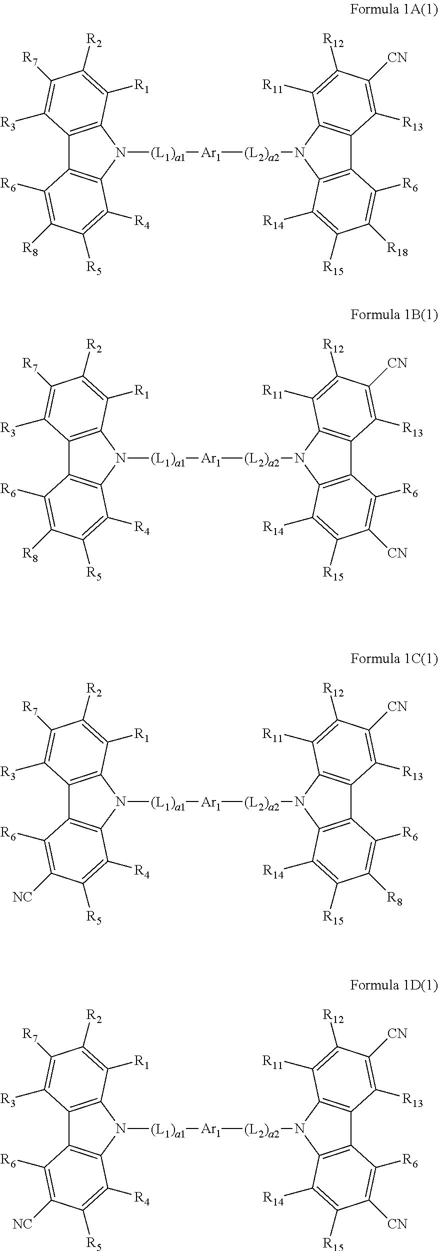

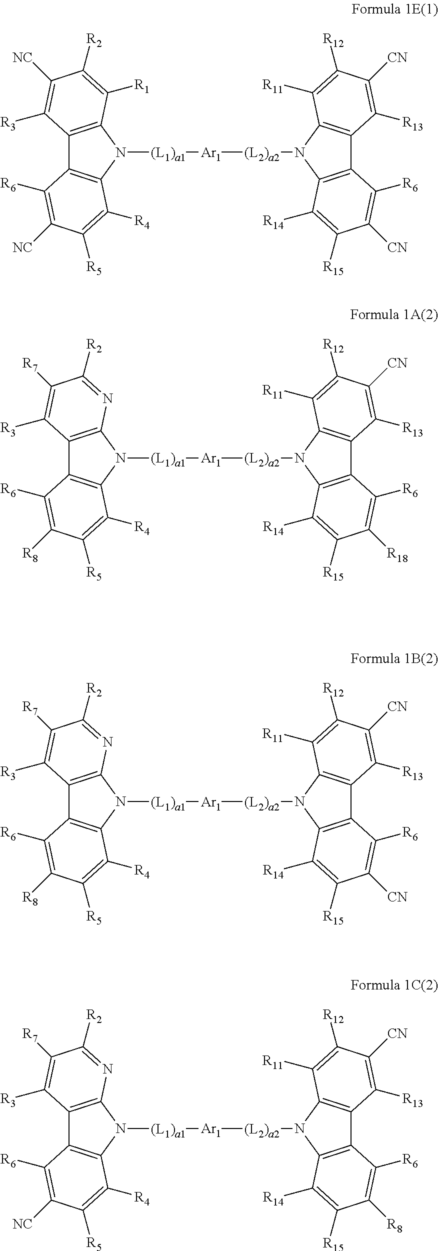

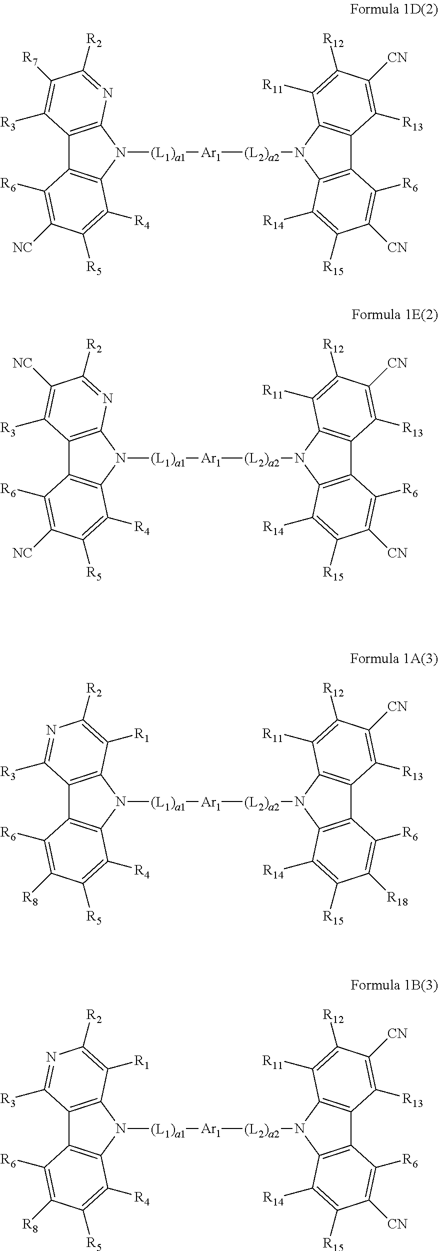

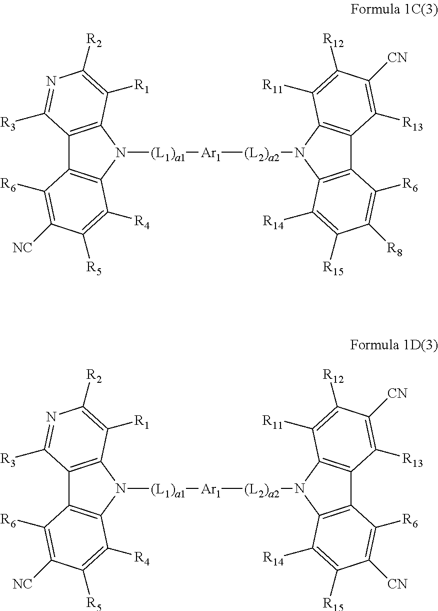

Descriptions of R₁ to R₈, R₁₁ to R₁₆, R₁₈, Ar₁, L₁, L₂, a1, a2 and *-(L₁)ₐ₁-Ar₁-(L₂)ₐ₂-*′ in Formulae 1A(1) to 1E(1), 1A(2) to 1E(2), and 1A(3) to 1D(3) are the same as presented above.

For example, each of R₁ to R₈, R₁₁ to R₁₆, and R₁₈ in Formulae 1 A(1) to 1E(1), 1A(2) to 1E(2), and 1A(3) to 1 D(3) may not be a cyano group.

In some embodiments, R₁ to R₈, R₁₁ to R₁₆, and R₁₈ in Formulae 1A(1) to 1E(1), 1A(2) to 1E(2), and 1A(3) to 1D(3) may be each independently selected from a hydrogen, a deuterium, —F, —Cl, —Br, —I, a hydroxyl group, a nitro group, an amino group, an amidino group, a hydrazine group, a hydrazone group, a carboxylic acid group or a salt thereof, a sulfonic acid group or a salt thereof, a phosphoric acid group or a salt thereof, a methyl group, an ethyl group, an n-propyl group, an isopropyl group, an n-butyl group, an isobutyl group, a sec-butyl group, a tert-butyl group, an n-pentyl group, an isopentyl group, a sec-pentyl group, a tert-pentyl group, an n-hexyl group, an iso-hexyl group, a sec-hexyl group, a tert-hexyl group, an n-heptyl group, an iso-heptyl group, a sec-heptyl group, a tert-heptyl group, an n-octyl group, an iso-octyl group, a sec-octyl group, a tert-octyl group, an n-nonyl group, an iso-nonyl group, a sec-nonyl group, a tert-nonyl group, an n-decyl group, an isodecyl group, a sec-decyl group, a tert-decyl group, a methoxy group, an ethoxy group, a propoxy group, a butoxy group, a pentoxy group, a phenyl group, a naphthyl group, a pyridinyl group, a pyrimidinyl group, a triazinyl group, and —Si(Q₁)(Q₂)(Q₃),

Ar₁ is represented by one of Formulae 2A-1, 2A-2, 2A-10, 2B-1, 2B-8, or 2C-3:

L₁ and L₂ may be each independently selected from

a phenylene group, a pyridinylene group, a pyrimidinylene group, a pyrazinylene group, a pyridazinylene group, a triazinylene group, a dibenzofuranylene group, and a dibenzothiophenylene group; and

a phenylene group, a pyridinylene group, a pyrimidinylene group, a pyrazinylene group, a pyridazinylene group, a triazinylene group, a dibenzofuranylene group, and a dibenzothiophenylene group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, a phenyl group, a naphthyl group, a pyridinyl group, a pyrimidinyl group, a pyrazinyl group, a pyridazinyl group, a triazinyl group and —Si(Q₂₁)(Q₂₂)(Q₂₃),

a1 and a2 may be each independently 0 or 1,

wherein Q₁ to Q₃, Q₁₁ to Q₁₃ and Q₂₁ to Q₂₃ may be each independently selected from a hydrogen, a C₁-C₁₀ alkyl group, a C₁-C₁₀ alkoxy group, and a phenyl group, but are not limited thereto.

In some embodiments,

i) the condensed cyclic compound may be represented by Formula 1A(1) in which Ar₁ is represented by one of Formulae 2A-2 to 2A-10, 2B-1 to 2B-8, and 2C-1 to 2C-9;

ii) the condensed cyclic compound may be represented by Formula 1A(1) in which the sum of a1 and a2 may not be 0; or

iii) the condensed cyclic compound may be represented by one of Formulae 1B(1) to 1E(1), 1A(2) to 1E(2), and 1A(3) to 1 D(3).

In some embodiments, Ar₁ in Formula 1 may be represented by Formula 2A-1, and when the sum of a1 and a2 is 0, at least two selected from X₇, X₈, X₁₇, and X₁₃ in Formula 1 may each be C(CN).

In an embodiment, the condensed cyclic compound represented by Formula 1 may be one of Compounds 1 to 56, but is not limited thereto:

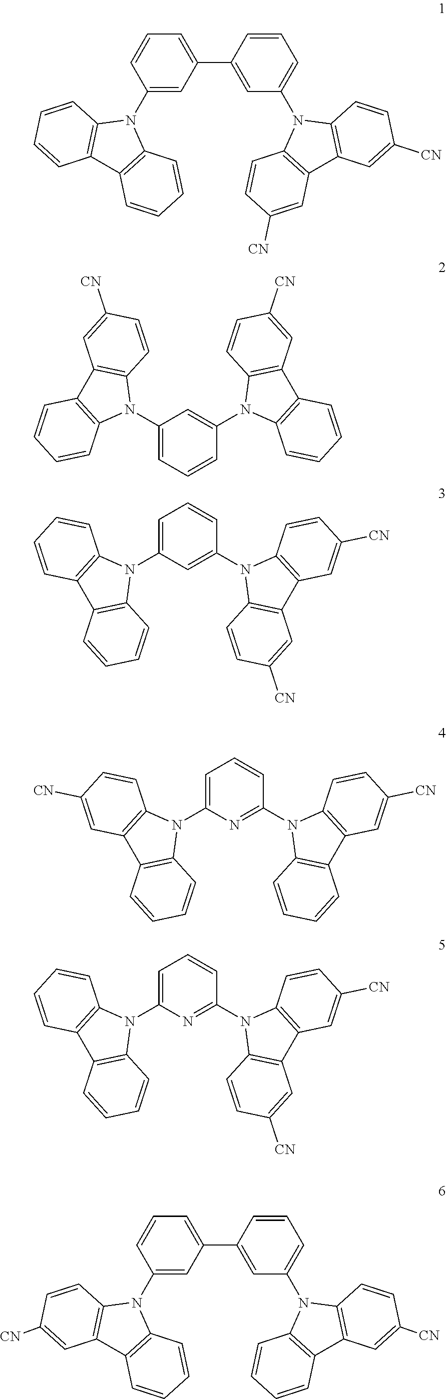

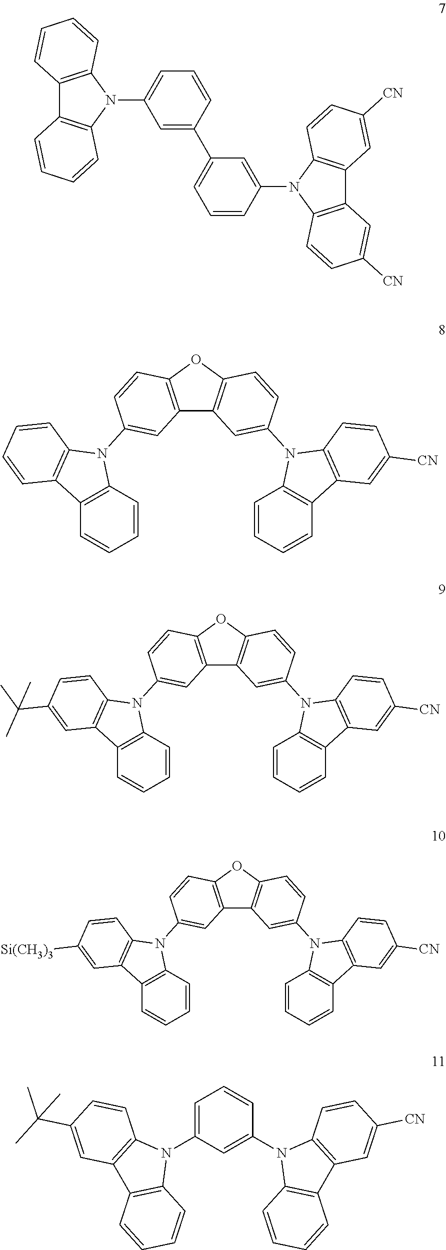

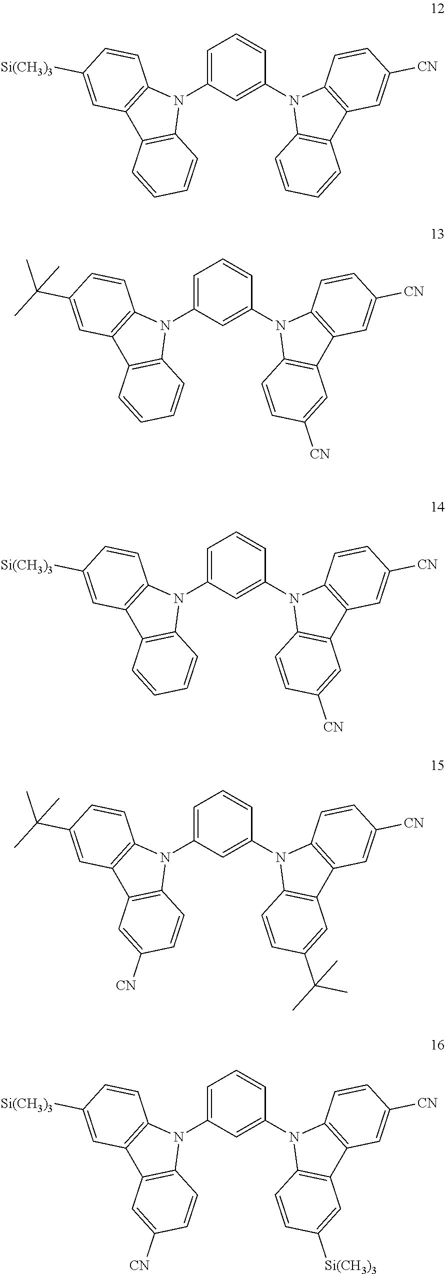

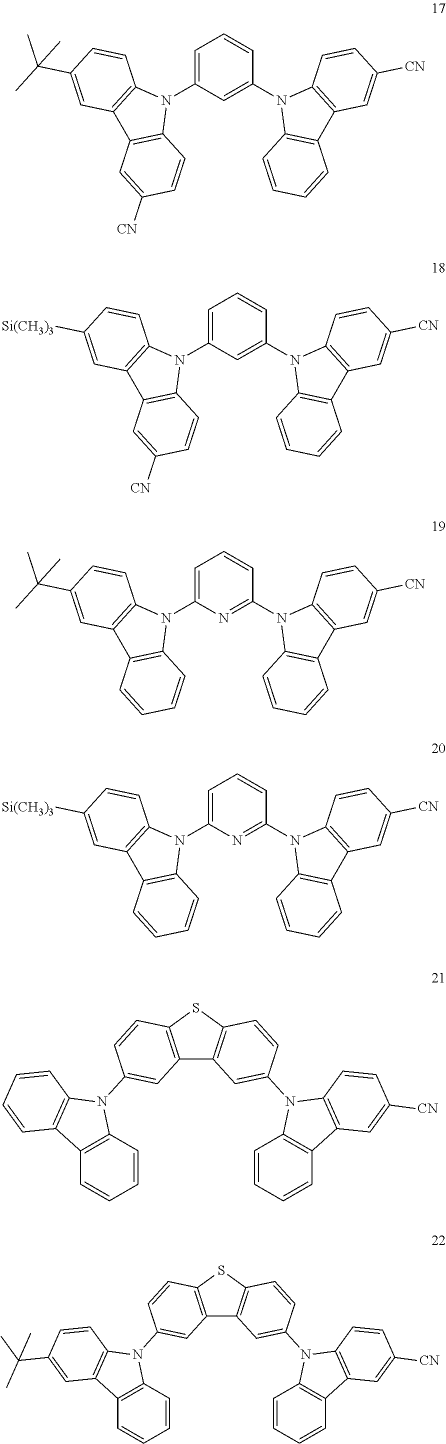

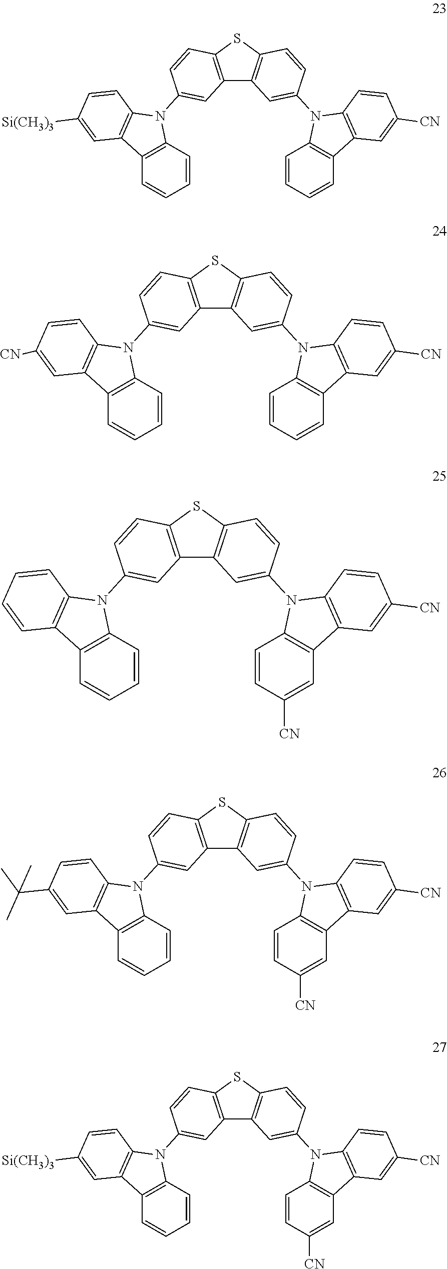

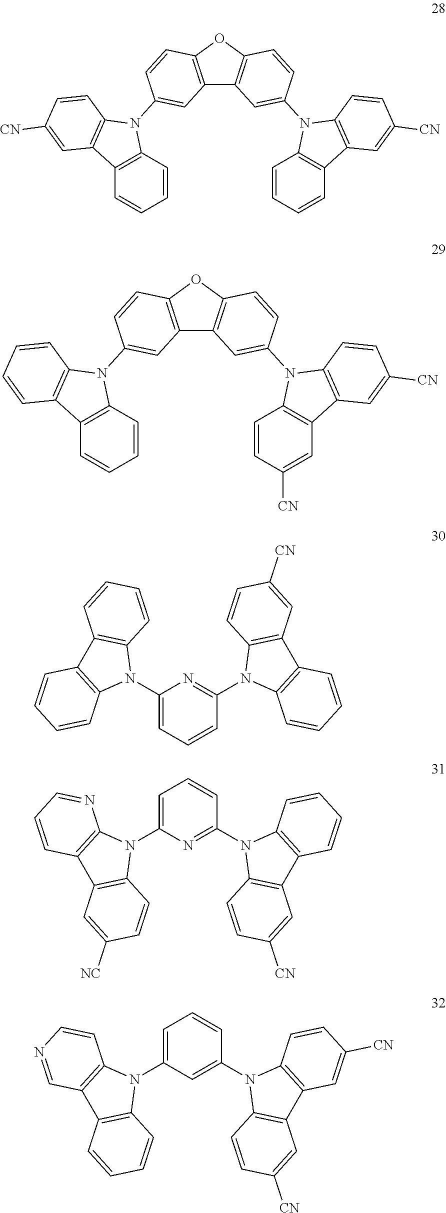

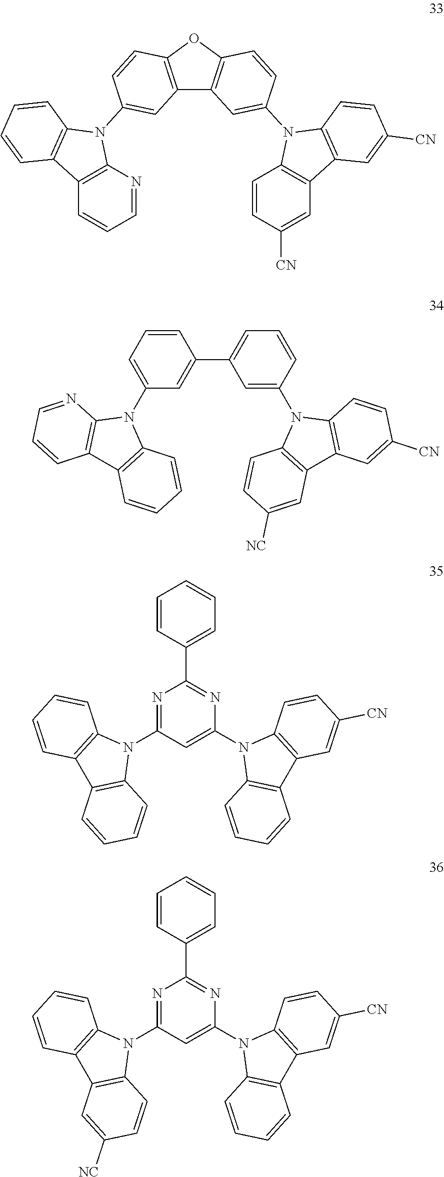 [SMILES: N#Cc1ccc2c(c1)c1cc(C#N)ccc1n2-c1ccc2oc3ccc(-n4c5ccccc5c5cccnc54)cc3c2c1 ; N#Cc1ccc2c(c1)c1cc(C#N)ccc1n2-c1cccc(-c2cccc(-n3c4ccccc4c4cccnc43)c2)c1 ; N#Cc1ccc2c(c1)c1ccccc1n2-c1cc(-n2c3ccccc3c3ccccc32)nc(-c2ccccc2)n1 ; N#Cc1ccc2c(c1)c1ccccc1n2-c1cc(-n2c3ccccc3c3cc(C#N)ccc32)nc(-c2ccccc2)n1]

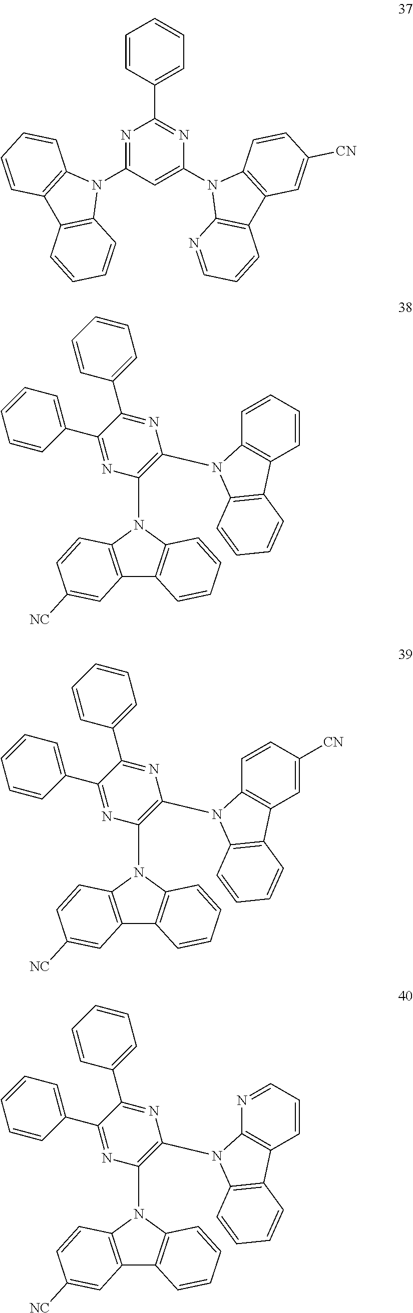

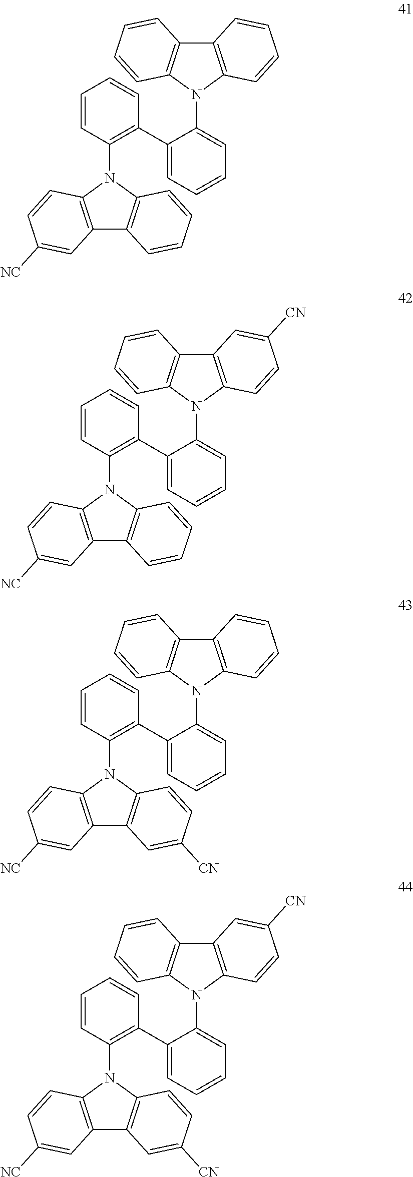 [SMILES: N#Cc1ccc2c(c1)c1ccccc1n2-c1ccccc1-c1ccccc1-n1c2ccccc2c2ccccc21 ; N#Cc1ccc2c(c1)c1ccccc1n2-c1ccccc1-c1ccccc1-n1c2ccccc2c2cc(C#N)ccc21 ; N#Cc1ccc2c(c1)c1cc(C#N)ccc1n2-c1ccccc1-c1ccccc1-n1c2ccccc2c2ccccc21 ; N#Cc1ccc2c(c1)c1ccccc1n2-c1ccccc1-c1ccccc1-n1c2ccc(C#N)cc2c2cc(C#N)ccc21]

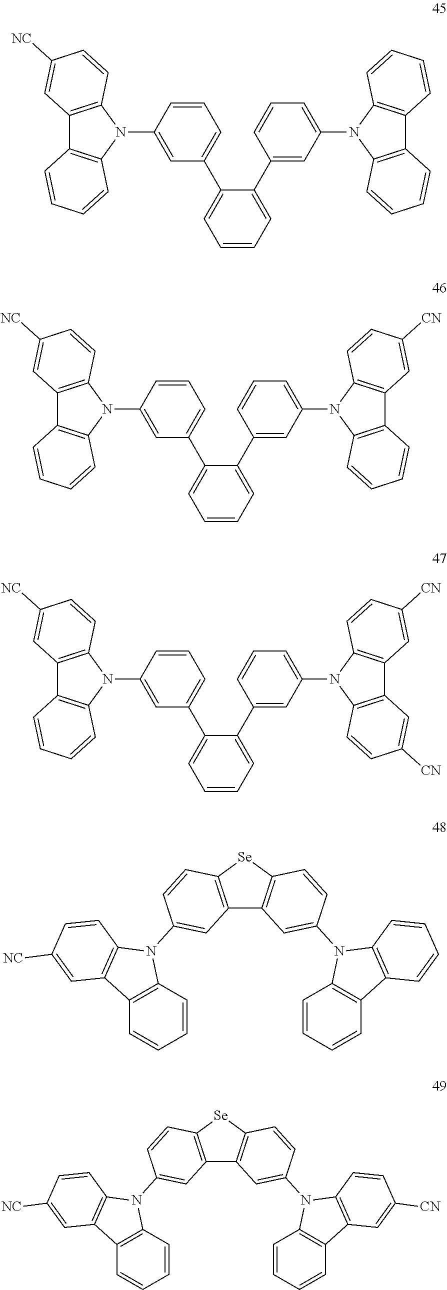 [SMILES: N#Cc1ccc2c(c1)c1ccccc1n2-c1cccc(-c2ccccc2-c2cccc(-n3c4ccccc4c4ccccc43)c2)c1 ; N#Cc1ccc2c(c1)c1ccccc1n2-c1cccc(-c2ccccc2-c2cccc(-n3c4ccccc4c4cc(C#N)ccc43)c2)c1 ; N#Cc1ccc2c(c1)c1ccccc1n2-c1cccc(-c2ccccc2-c2cccc(-n3c4ccc(C#N)cc4c4cc(C#N)ccc43)c2)c1 ; N#Cc1ccc2c(c1)c1ccccc1n2-c1ccc2[se]c3ccc(-n4c5ccccc5c5ccccc54)cc3c2c1 ; N#Cc1ccc2c(c1)c1ccccc1n2-c1ccc2[se]c3ccc(-n4c5ccccc5c5cc(C#N)ccc54)cc3c2c1]

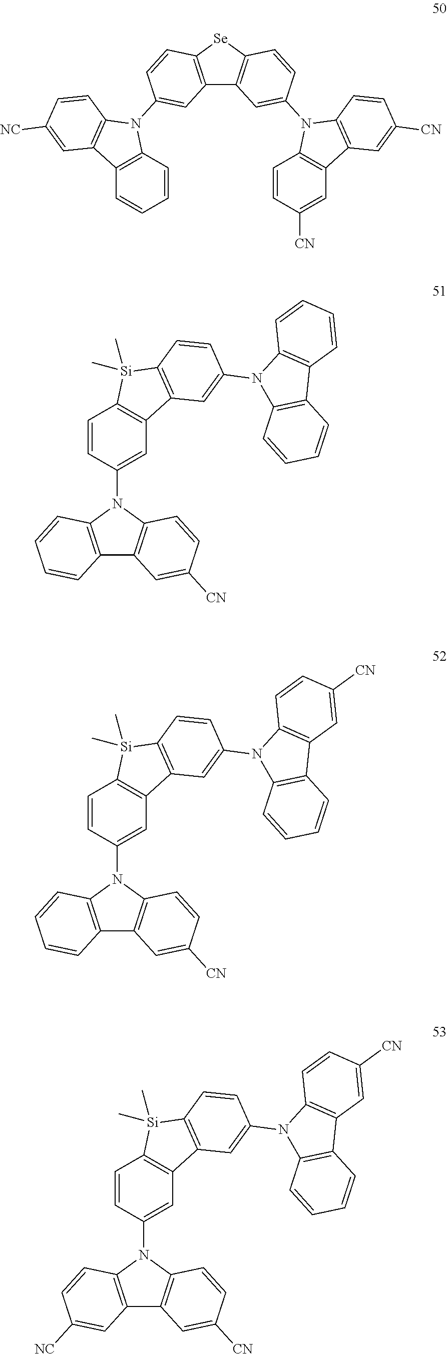 [SMILES: N#Cc1ccc2c(c1)c1ccccc1n2-c1ccc2[se]c3ccc(-n4c5ccc(C#N)cc5c5cc(C#N)ccc54)cc3c2c1 ; C[Si]1(C)c2ccc(-n3c4ccccc4c4ccccc43)cc2-c2cc(-n3c4ccccc4c4cc(C#N)ccc43)ccc21 ; C[Si]1(C)c2ccc(-n3c4ccccc4c4cc(C#N)ccc43)cc2-c2cc(-n3c4ccccc4c4cc(C#N)ccc43)ccc21 ; C[Si]1(C)c2ccc(-n3c4ccccc4c4cc(C#N)ccc43)cc2-c2cc(-n3c4ccc(C#N)cc4c4cc(C#N)ccc43)ccc21]

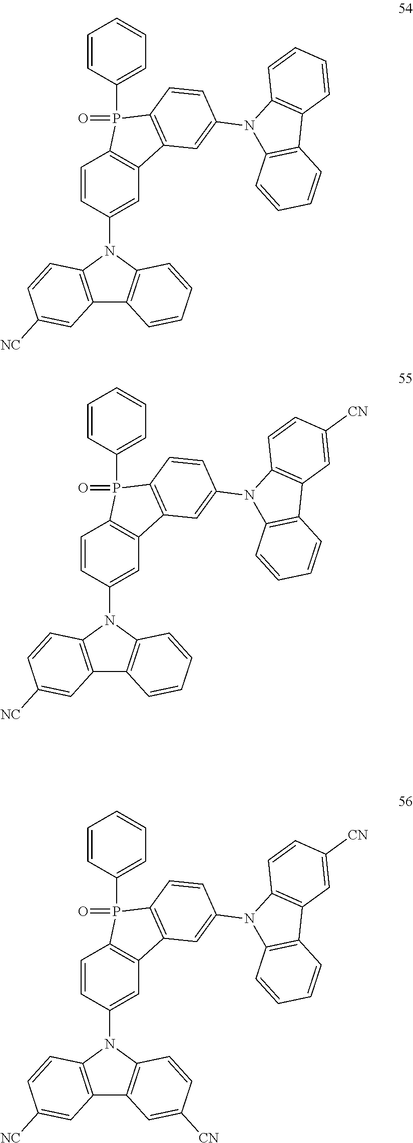 [SMILES: N#Cc1ccc2c(c1)c1ccccc1n2-c1ccc2c(c1)-c1cc(-n3c4ccccc4c4ccccc43)ccc1P2(=O)c1ccccc1 ; N#Cc1ccc2c(c1)c1ccccc1n2-c1ccc2c(c1)-c1cc(-n3c4ccccc4c4cc(C#N)ccc43)ccc1P2(=O)c1ccccc1 ; N#Cc1ccc2c(c1)c1ccccc1n2-c1ccc2c(c1)-c1cc(-n3c4ccc(C#N)cc4c4cc(C#N)ccc43)ccc1P2(=O)c1ccccc1]

At least one selected from R₇, R₈, R₁₇, and R₁₈ in the condensed cyclic compound represented by Formula 1 may be C(CN), the condensed cyclic compound represented by Formula 1 includes a linking group represented by “Ar₁”, which is represented by one of Formulae 2A to 2C and the condensed cyclic compound represented by Formula 1 is not following compound:

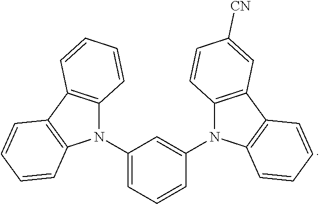 [SMILES: N#Cc1ccc2c(c1)c1ccccc1n2-c1cccc(-n2c3ccccc3c3ccccc32)c1]

Accordingly the condensed cyclic compound represented by Formula 1 may have excellent heat resistance and high triplet energy level.

In some embodiments, in the condensed cyclic compound represented by Formula 1, the list of R₁ to R₈ and R₁₁ to R₁₈ does not include “a substituted or unsubstituted carbazolyl group,” the list of Ar₁, L₁ and L₂ does not include “a substituted or unsubstituted carbazolylene group,” and the list of Ar₁, L₁ and L₂ does not include “a substituted or unsubstituted carbazolyl group.” That is, the condensed cyclic compound represented by Formula 1 has, as a carbazole ring, a carbazole ring 1, and a carbazole ring 2, as shown in Formula 1′ below. Accordingly, the condensed cyclic compound represented by Formula 1 may have a triplet (T₁) energy level that is suitable for an electronic device, for example, for use as a material for an organic light-emitting device (for example, a host material in an emission layer).

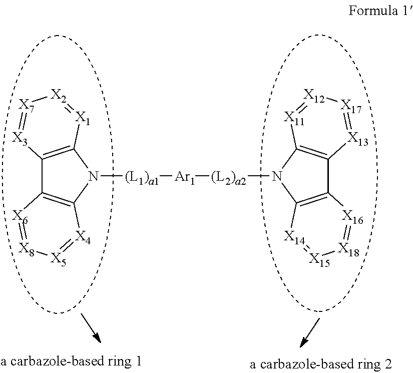

The condensed cyclic compound represented by Formula 1 may have a relatively small difference between S₁ (singlet) energy and T₁ (triplet) energy. Accordingly, the condensed cyclic compound represented by Formula 1 may be used as a thermally activated delayed fluorescence emitter (TADF emitter).

For example, the highest occupied molecular orbital (HOMO), lowest unoccupied molecular orbital (LUMO), T₁ and S₁ energy levels of Compounds 1 to 30 and A were simulated by using Gaussian program DFT method (the structure is optimized at B3LYP, 6-31G(d,p) level), and simulation evaluation results are shown in Table 1 below:

TABLE 1

| | HOMO | LUMO | T₁ | S₁ |
|---|---|---|---|---|
| | (eV) | (eV) | (eV) | (eV) |
| Compound 1 | −5.594 | −1.654 | 3.080 | 3.435 |
| Compound 2 | −6.08 | −1.44 | 3.03 | 3.33 |
| Compound 3 | −6.08 | −1.44 | 3.03 | 3.33 |
| Compound 4 | −6.06 | −1.72 | 3.03 | 3.24 |
| Compound 5 | −5.929 | −1.700 | 3.085 | 3.606 |
| Compound 6 | −5.93 | −1.63 | 3.12 | 3.76 |
| Compound 7 | −5.594 | −1.654 | 3.080 | 3.435 |
| Compound 8 | −5.51 | −1.68 | 3.09 | 3.30 |
| Compound 9 | −5.42 | −1.66 | 3.04 | 3.23 |
| Compound 10 | −5.49 | −1.68 | 3.08 | 3.29 |
| Compound 11 | −5.54 | −1.24 | 3.12 | 3.85 |
| Compound 12 | −5.62 | −1.25 | 3.12 | 3.89 |
| Compound 13 | −5.71 | −1.69 | 3.08 | 3.63 |
| Compound 14 | −5.78 | −1.70 | 3.08 | 3.70 |
| Compound 15 | −5.90 | −1.37 | 3.10 | 3.94 |
| Compound 16 | −6.03 | −1.41 | 3.11 | 3.99 |
| Compound 17 | −5.95 | −1.41 | 3.10 | 3.95 |
| Compound 18 | −6.03 | −1.42 | 3.11 | 3.99 |
| Compound 19 | −5.60 | −1.38 | 3.11 | 3.61 |
| Compound 20 | −5.67 | −1.41 | 3.11 | 3.64 |
| Compound 21 | −5.50 | −1.66 | 3.06 | 3.30 |
| Compound 22 | −5.41 | −1.64 | 3.03 | 3.24 |
| Compound 23 | −5.49 | −1.66 | 3.06 | 3.30 |
| Compound 24 | −5.92 | −1.88 | 3.09 | 3.50 |
| Compound 25 | −5.62 | −1.87 | 3.01 | 3.22 |
| Compound 26 | −5.52 | −1.85 | 2.97 | 3.15 |
| Compound 27 | −5.60 | −1.87 | 3.01 | 3.21 |
| Compound 28 | −5.956 | −1.905 | 3.121 | 3.296 |
| Compound 29 | −5.615 | −1.887 | 3.025 | 3.203 |
| Compound 30 | −5.733 | −1.438 | 3.117 | 3.553 |
| Compound A | −5.450 | −1.080 | 3.160 | 3.330 |

Compound A

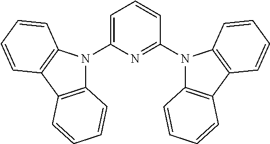 [SMILES: c1cc(-n2c3ccccc3c3ccccc32)nc(-n2c3ccccc3c3ccccc32)c1]

A synthesis method for the condensed cyclic compound represented by Formula 1 may be understood by one of ordinary skill in the art by referring to Synthesis Examples.

Accordingly, the condensed cyclic compound represented by Formula 1 is suitable for an organic layer of an organic light-emitting device, for example, for use as a host or emitter (for example, a TADF emitter) of an emission layer in the organic layer. Thus, in another aspect, provided is an organic light-emitting device including:

a first electrode;

a second electrode; and

an organic layer disposed between the first electrode and the second electrode,

wherein the organic layer including an emission layer and at least one of the condensed cyclic compounds represented by Formula 1.

Due to the inclusion of the condensed cyclic compound represented by Formula 1, the organic light-emitting device may have a low driving voltage, high efficiency, high luminance, high quantum luminescent efficiency, and long lifespan.

The condensed cyclic compound represented by Formula 1 may be used between a pair of electrodes that constitute an organic light-emitting device. For example, the condensed cyclic compound may be included in at least one selected from an emission layer, a hole transport region (for example, including at least one selected from a hole injection layer, a hole transport layer, and an electron blocking layer) disposed between a first electrode and the emission layer, and an electron transport region (for example, including at least one selected from a hole blocking layer, an electron transport layer, and an electron injection layer) disposed between the emission layer and a second electrode.

For example, the condensed cyclic compound represented by Formula 1 may be included in the emission layer. The condensed cyclic compound included in the emission layer may act as a host, and the emission layer may further include a dopant (a fluorescent dopant or a phosphorescent dopant). The emission layer may be a green emission layer emitting green light or a blue emission layer emitting blue light. In an embodiment, the condensed cyclic compound represented by Formula 1 may be included in the emission layer, the emission layer may further include a phosphorescent dopant, and the emission layer may emit blue light.

In some embodiments, the condensed cyclic compound represented by Formula 1 may be included in the emission layer, and the condensed cyclic compound may be a TADF emitter. In some embodiments, the emission layer may include the condensed cyclic compound represented by Formula 1 alone. In some embodiments, the emission layer may further include, in addition to the condensed cyclic compound represented by Formula 1, a host and/or a dopant.

The term “organic layer” as used herein refers to a single layer and/or a plurality of layers between the first electrode and the second electrode of an organic light-emitting device. The “organic layer” may include, in addition to an organic compound, an organometallic complex including metal.

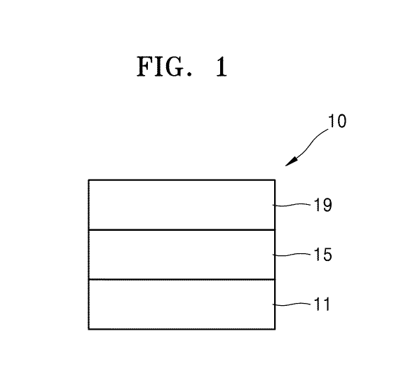

FIG. 1 is a schematic view of an organic light-emitting device 10 according to an embodiment. Hereinafter, the structure of an organic light-emitting device according to an embodiment and a method of manufacturing an organic light-emitting device according to an embodiment will be described in connection with FIG. 1. The organic light-emitting device 10 includes a first electrode 11, an organic layer 15, and a second electrode 19, which are sequentially stacked.

In FIG. 1, a substrate may be additionally disposed under the first electrode 11 or above the second electrode 19. For use as the substrate, any substrate that is used in general organic light-emitting devices may be used, and the substrate may be a glass substrate or transparent plastic substrate, each with excellent mechanical strength, thermal stability, transparency, surface smoothness, ease of handling, and water-resistance.

The first electrode 11 may be formed by depositing or sputtering a material for forming the first electrode on the substrate. The first electrode 11 may be an anode. The material for the first electrode 11 may be selected from materials with a high work function to allow holes be easily provided. The first electrode 11 may be a reflective electrode or a transmissive electrode. The material for the first electrode may be, for example, indium tin oxide (ITO), indium zinc oxide (IZO), tin oxide (SnO₂), and zinc oxide (ZnO). In some embodiments, magnesium (Mg), aluminum (Al), aluminum-lithium (Al—Li), calcium (Ca), magnesium-indium (Mg—In), or magnesium-silver (Mg—Ag) may be used as the material for the first electrode.

The first electrode 11 may have a single-layer structure or a multi-layer structure including two or more layers. For example, the first electrode 11 may have a three-layered structure of ITO/Ag/ITO, but the structure of the first electrode 110 is not limited thereto.

An organic layer 15 is disposed on the first electrode 11.

The organic layer 15 may include a hole transport region, an emission layer, and an electron transport region.

The hole transport region may be disposed between the first electrode 11 and the emission layer.

The hole transport region may include at least one selected from a hole injection layer, a hole transport layer, an electron blocking layer, and a buffer layer.

The hole transport region may include only either a hole injection layer or a hole transport layer. In some embodiments, the hole transport region may have a structure of hole injection layer/hole transport layer or hole injection layer/hole transport layer/electron blocking layer, which are sequentially stacked in this stated order from the first electrode 11.

A hole injection layer hole injection layer may be formed on the first electrode 11 by using various methods, such as vacuum deposition, spin coating, casting, or Langmuir-Blodgett (LB).

When a hole injection layer is formed by vacuum deposition, the deposition conditions may vary according to a material that is used to form the hole injection layer, and the structure and thermal characteristics of the hole injection layer. For example, the deposition conditions may include a deposition temperature of about 100 to about 500° C., a vacuum pressure of about 10⁻⁸ to about 10⁻³ torr, and a deposition rate of about 0.01 to about 100 Angstroms per second (A/sec). However, the deposition conditions are not limited thereto.

When the hole injection layer is formed using spin coating, coating conditions may vary according to the material used to form the hole injection layer, and the structure and thermal properties of the hole injection layer. For example, a coating speed may be from about 2,000 revolutions per minute (rpm) to about 5,000 rpm, and a temperature at which a heat treatment is performed to remove a solvent after coating may be from about 80° C. to about 200° C. However, the coating conditions are not limited thereto.

Conditions for a hole transport layer and an electron blocking layer may be understood by referring to conditions for forming the hole injection layer.

The hole transport region may include at least one selected from m-MTDATA, TDATA, 2-TNATA, NPB, β-NPB, TPD, Spiro-TPD, Spiro-NPB, methylated-NPB, TAPC, HMTPD, 4,4′,4″-tris(N-carbazolyl)triphenylamine (TCTA), polyaniline/dodecylbenzenesulfonic acid (Pani/DBSA), poly(3,4-ethylenedioxythiophene)/poly(4-styrenesulfonate) (PEDOT/PSS), polyaniline/camphor sulfonic acid (PANI/CSA), (polyaniline)/poly(4-styrenesulfonate) (Pani/PSS), a compound represented by Formula 201 below, and a compound represented by Formula 202 below:

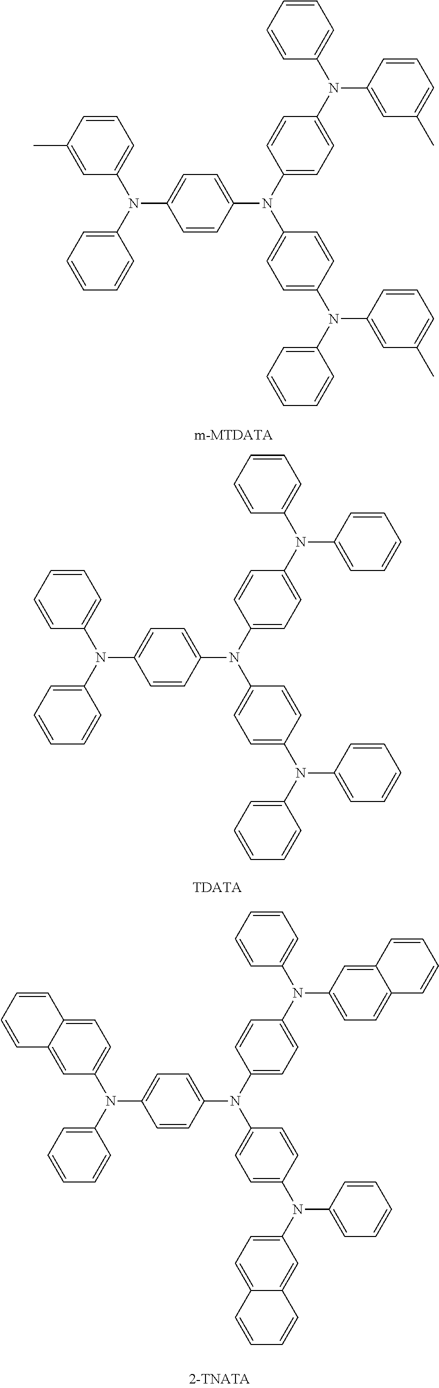

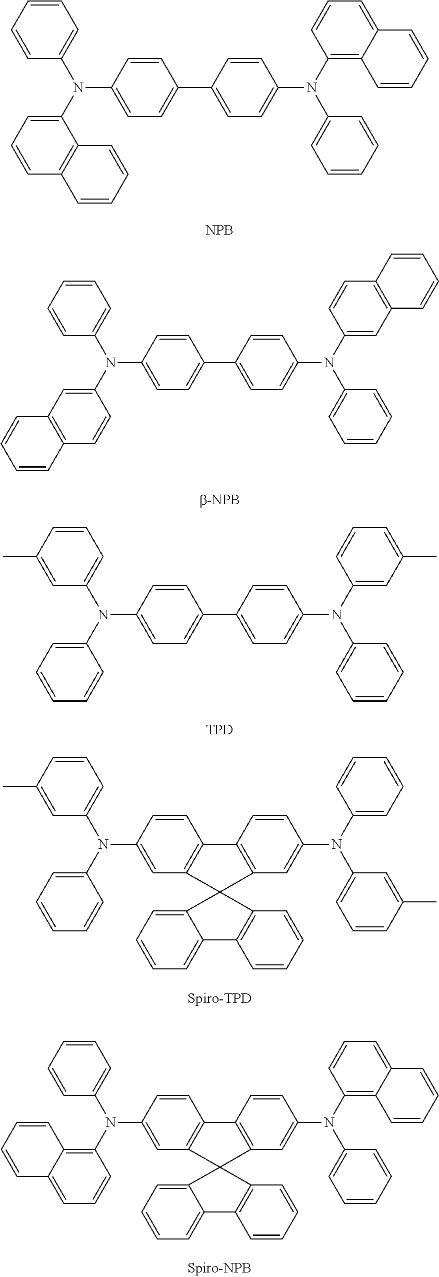

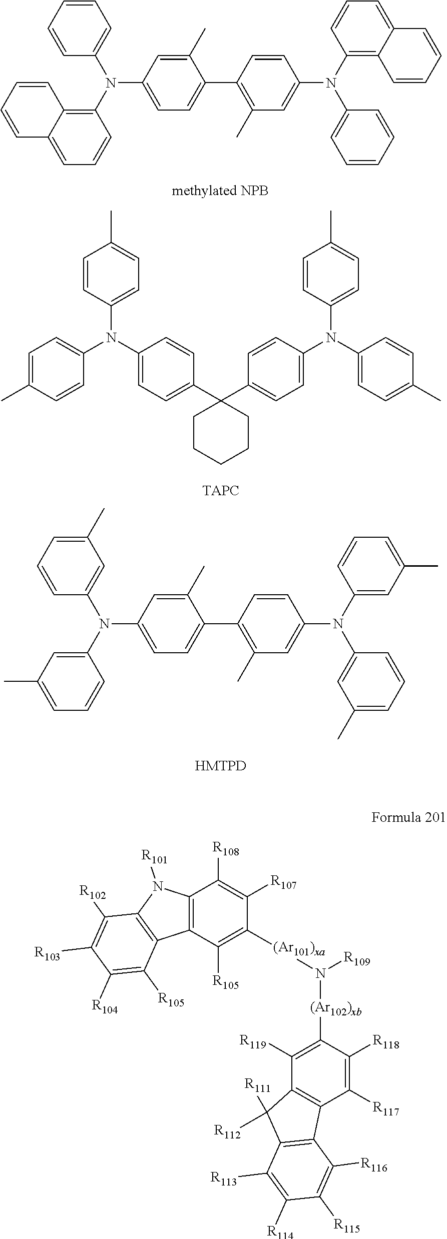

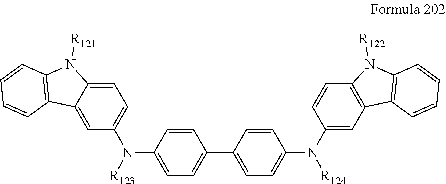

Ar₁₀₁ to Ar₁₀₂ in Formula 201 may be each independently selected from

a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, and a pentacenylene group; and

a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, and a pentacenylene group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₃-C₁₀ cycloalkenyl group, a C₁-C₁₀ heterocycloalkyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, and a monovalent non-aromatic condensed heteropolycyclic group.

In Formula 201, xa and xb may be each independently an integer of 0 to 5, or 0, 1, or 2. For example, xa is 1 and xb is 0, but xa and xb are not limited thereto.

R₁₀₁ to R₁₀₃, R₁₁₁ to R₁₁₉ and R₁₂₁ to R₁₂₄ in Formulae 201 and 202 may be each independently selected from a hydrogen, a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group (for example, a methyl group, an ethyl group, a propyl group, a butyl group, a pentyl group, a hexyl group, and so on), or a C₁-C₁₀ alkoxy group (for example, a methoxy group, an ethoxy group, a propoxy group, a butoxy group, a pentoxy group, and so on);

a C₁-C₁₀ alkyl group or a C₁-C₁₀ alkoxy group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, and a phosphoric acid group or a salt thereof;

a phenyl group, a naphthyl group, an anthracenyl group, a fluorenyl group, and a pyrenyl group; and

a phenyl group, a naphthyl group, an anthracenyl group, a fluorenyl group, and a pyrenyl group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, and a C₁-C₁₀ alkoxy group,

but they are not limited thereto.

R₁₀₉ in Formula 201 may be selected from

a phenyl group, a naphthyl group, an anthracenyl group, and a pyridinyl group; and

a phenyl group, a naphthyl group, an anthracenyl group, and a pyridinyl group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, an anthracenyl group, and a pyridinyl group.

According to an embodiment, the compound represented by Formula 201 may be represented by Formula 201A, but is not limited thereto:

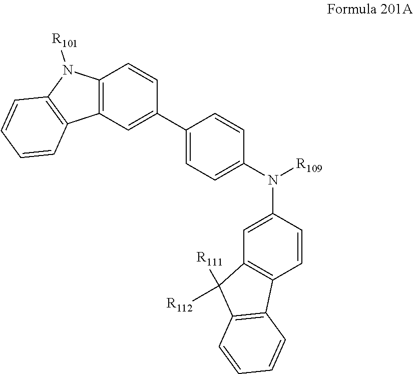

R₁₀₁, R₁₁₁, R₁₁₂, and R₁₀₉ in Formula 201A may be understood by referring to the description provided herein.

For example, the compound represented by Formula 201, and the compound represented by Formula 202 may include Compounds HT1 to HT20 illustrated below, but are not limited thereto.

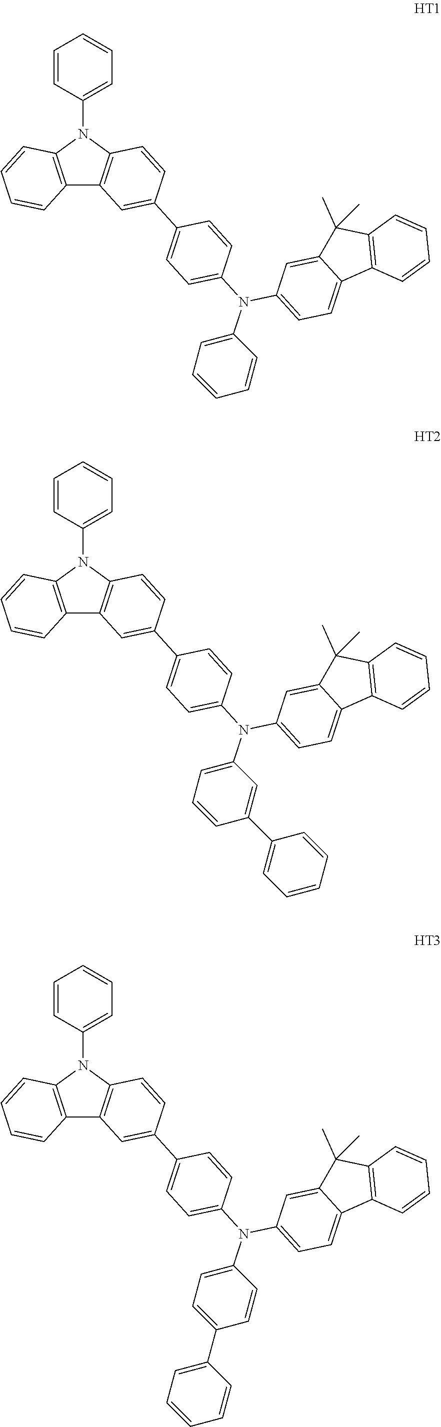 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

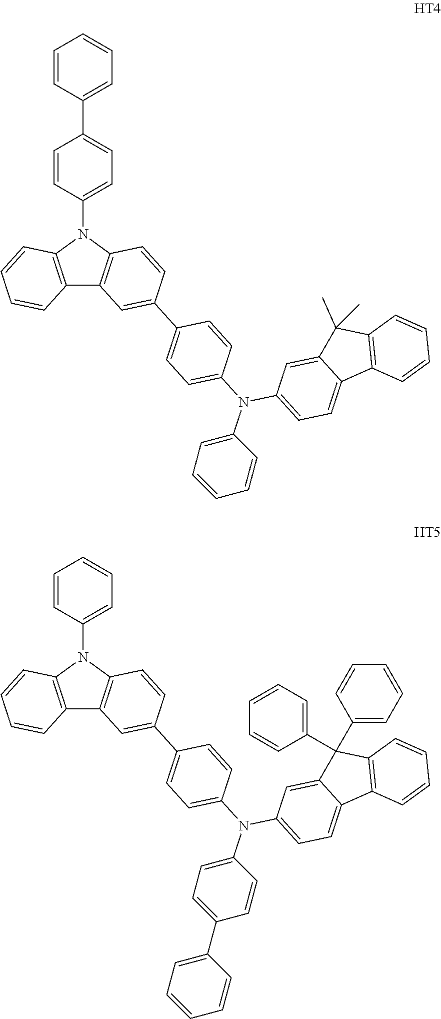 [SMILES: CC1(C)c2ccccc2-c2ccc(N(c3ccccc3)c3ccc(-c4ccc5c(c4)c4ccccc4n5-c4ccc(-c5ccccc5)cc4)cc3)cc21 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

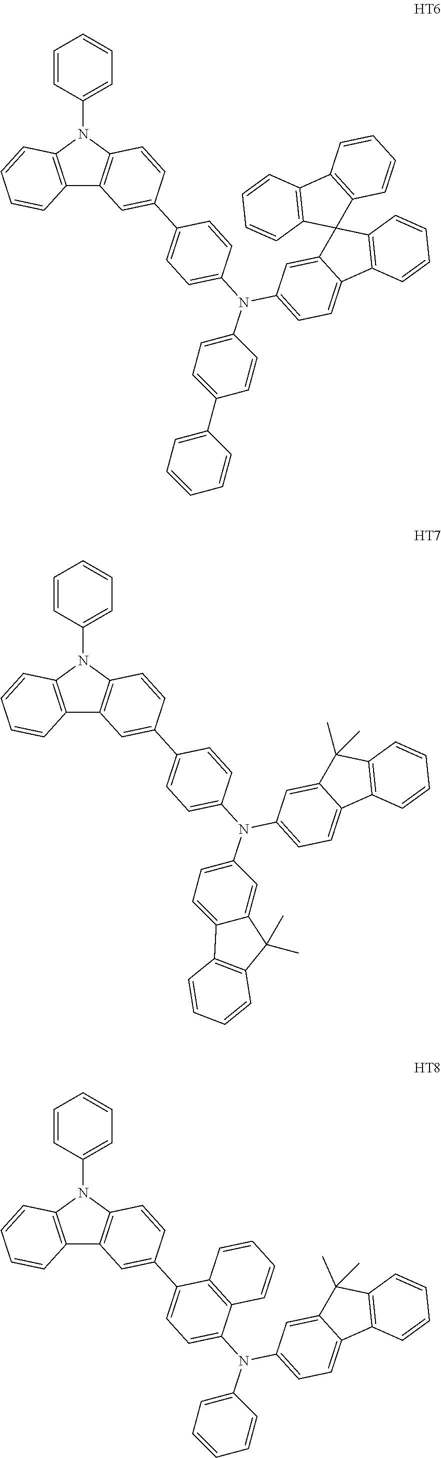 [SMILES: c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1 ; CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3ccccc3)c3ccc(-c4ccc5c(c4)c4ccccc4n5-c4ccccc4)c4ccccc34)cc21]

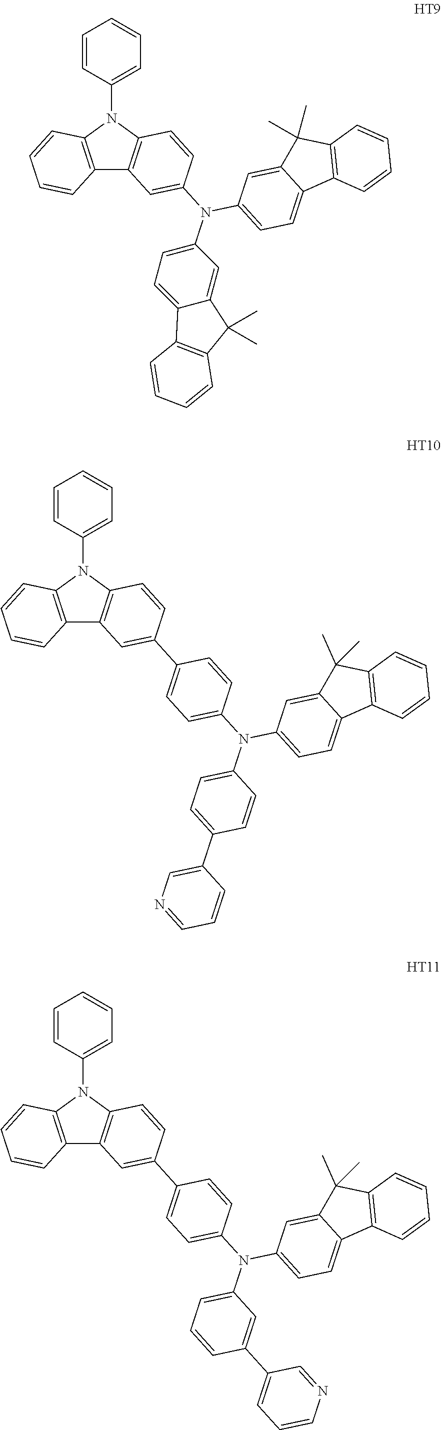 [SMILES: CC1(C)c2ccccc2-c2ccc(N(c3ccc4c(c3)C(C)(C)c3ccccc3-4)c3ccc4c(c3)c3ccccc3n4-c3ccccc3)cc21 ; CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21]

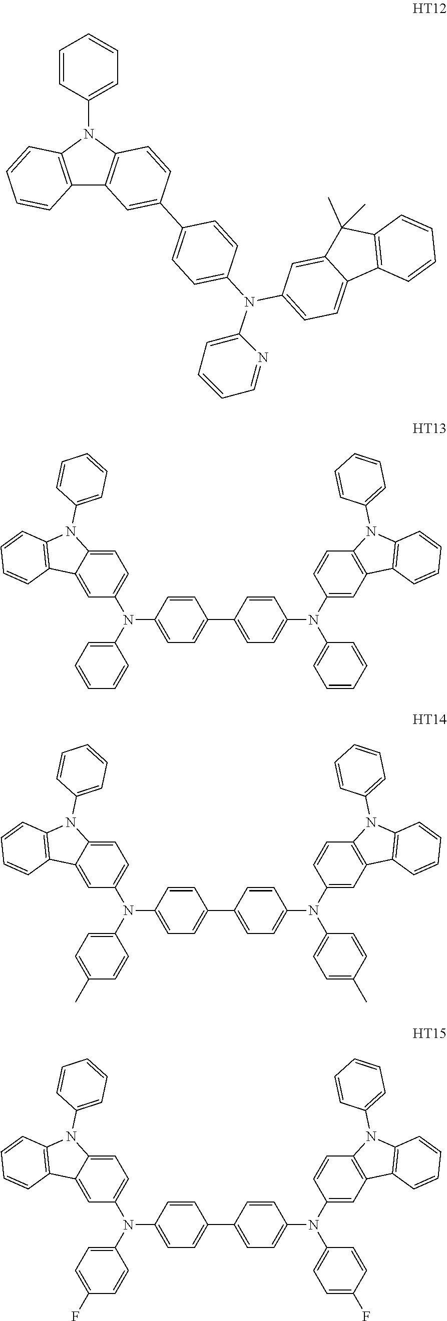 [SMILES: CC1(C)c2ccccc2-c2ccc(N(c3ccc(-c4ccc5c(c4)c4ccccc4n5-c4ccccc4)cc3)c3ccccn3)cc21 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; Cc1ccc(N(c2ccc(-c3ccc(N(c4ccc(C)cc4)c4ccc5c(c4)c4ccccc4n5-c4ccccc4)cc3)cc2)c2ccc3c(c2)c2ccccc2n3-c2ccccc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1]

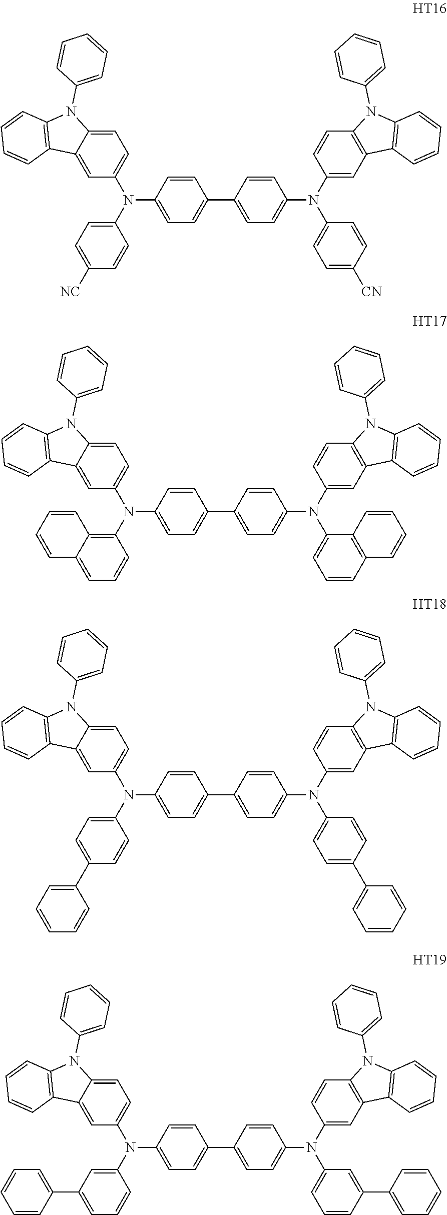 [SMILES: N#Cc1ccc(N(c2ccc(-c3ccc(N(c4ccc(C#N)cc4)c4ccc5c(c4)c4ccccc4n5-c4ccccc4)cc3)cc2)c2ccc3c(c2)c2ccccc2n3-c2ccccc2)cc1 ; c1ccc(-n2c3ccccc3c3cc(N(c4ccc(-c5ccc(N(c6ccc7c(c6)c6ccccc6n7-c6ccccc6)c6cccc7ccccc67)cc5)cc4)c4cccc5ccccc45)ccc32)cc1 ; c1ccc(-c2ccc(N(c3ccc(-c4ccc(N(c5ccc(-c6ccccc6)cc5)c5ccc6c(c5)c5ccccc5n6-c5ccccc5)cc4)cc3)c3ccc4c(c3)c3ccccc3n4-c3ccccc3)cc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1]

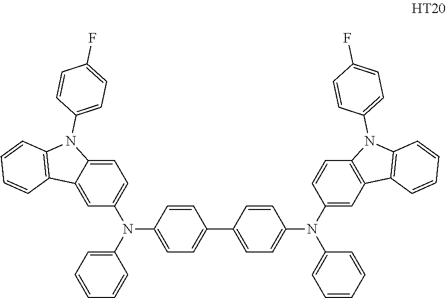 [SMILES: Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1]

A thickness of the hole transport region may be in a range of about 100 Angstroms (Å) to about 10,000 Å, for example, about 100 Å to about 1,000 Å. When the hole transport region includes a hole injection layer and a hole transport layer, the thickness of the hole injection layer may be in a range of about 100 Å to about 10,000 Å, and for example, about 100 Å to about 1,000 Å, and the thickness of the hole transport layer may be in a range of about 50 Å to about 2,000 Å, and for example, about 100 Å to about 1,500 Å. While not wishing to be bound by a theory, it is understood that when the thicknesses of the hole transport region, the hole injection layer, and the hole transport layer are within these ranges, satisfactory hole transporting characteristics may be obtained without a substantial increase in driving voltage.

The hole transport region may further include, in addition to these materials, a charge-generation material for the improvement of conductive properties. The charge-generation material may be homogeneously or non-homogeneously dispersed in the hole transport region.

The charge-generation material may be, for example, a p-dopant. The p-dopant may be one selected from a quinone derivative, a metal oxide, and a cyano group-containing compound, but embodiments are not limited thereto. Non-limiting examples of the p-dopant are a quinone derivative, such as tetracyanoquinonedimethane (TCNQ) or 2,3,5,6-tetrafluoro-tetracyano-1,4-benzoquinonedimethane (F4-TCNQ); a metal oxide, such as a tungsten oxide or a molybdenum oxide; and a cyano group-containing compound, such as Compound HT-D1 or HP-1, but are not limited thereto.

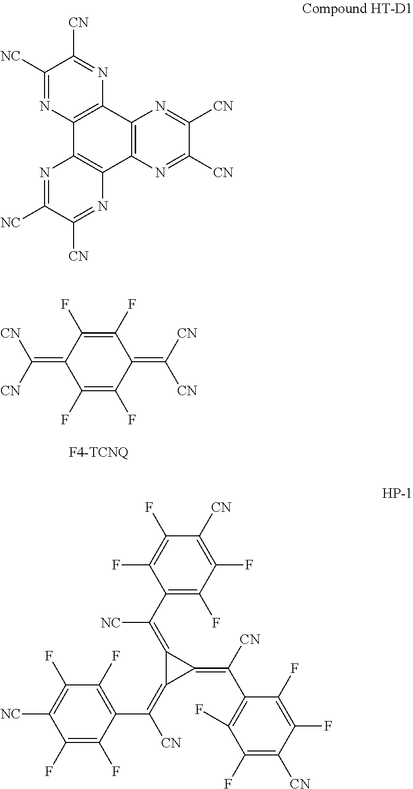

The hole transport region may include a buffer layer.

Also, the buffer layer may compensate for an optical resonance distance according to a wavelength of light emitted from the emission layer, and thus, efficiency of a formed organic light-emitting device may be improved.

Then, an emission layer (EML) may be formed on the hole transport region by vacuum deposition, spin coating, casting, LB deposition, or the like. When the emission layer is formed by vacuum deposition or spin coating, the deposition or coating conditions may be similar to those applied to form the hole injection layer although the deposition or coating conditions may vary according to the material that is used to form the emission layer.

The electron transport region may further include an electron blocking layer. The electron blocking layer may include, for example, mCP, but a material therefor is not limited thereto.

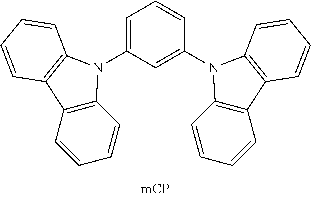 [SMILES: c1cc(-n2c3ccccc3c3ccccc32)cc(-n2c3ccccc3c3ccccc32)c1]

When the organic light-emitting device is a full color organic light-emitting device, the emission layer may be patterned into a red emission layer, a green emission layer, and a blue emission layer. In some embodiments, due to a stack structure including a red emission layer, a green emission layer, and/or a blue emission layer, the emission layer may emit white light.

The emission layer may include the condensed cyclic compound represented by Formula 1. The emission layer may include a dopant. The dopant may be at least one selected from a phosphorescent dopant and a fluorescent dopant.

In some embodiments, the emission layer may include the condensed cyclic compound represented by Formula 1 alone, and the condensed cyclic compound may be a TADF emitter.

In some embodiments, the emission layer may include the condensed cyclic compound represented by Formula 1, the condensed cyclic compound may be a TADF emitter, and the emission layer may further include a host.

For example, a host in the emission layer may include the condensed cyclic compound represented by Formula 1.

A dopant in the emission layer may be a fluorescent dopant that emits light according to a fluorescent emission mechanism or a phosphorescent dopant that emits light according to a phosphorescent emission mechanism.

According to an embodiment, the dopant in the emission layer may be a phosphorescent dopant, and the phosphorescent dopant may include an organometallic compound represented by Formula 81 below:

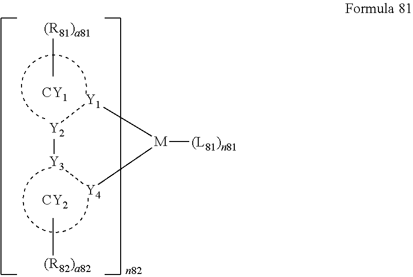

wherein in Formula 81,

M may be selected from iridium (Ir), platinum (Pt), osmium (Os), titanium (Ti), zirconium (Zr), hafnium (Hf), europium (Eu), terbium (Tb), and thulium (Tm);

Y₁ to Y₄ are each independently carbon (C) or nitrogen (N);

Y₁ and Y₂ are linked via a single bond or a double bond, and Y₃ and Y₄ are linked via a single bond or a double bond;

CY₁ and CY₂ are each independently selected from a benzene, a naphthalene, a fluorene, a spiro-fluorene, an indene, a pyrrole, a thiophene, a furan, an imidazole, a pyrazole, a thiazole, an isothiazole, an oxazole, an isoxazole, a pyridine, a pyrazine, a pyrimidine, a pyridazine, a quinoline, an isoquinoline, a benzoquinoline, a quinoxaline, a quinazoline, a carbazole, a benzoimidazole, a benzofuran, a benzothiophene, an isobenzothiophene, a benzoxazole, an isobenzoxazole, a triazole, a tetrazole, an oxadiazole, a triazine, a dibenzofuran, and a dibenzothiophene, and CY₁ and CY₂ are optionally linked to each other through a single bond or an organic linking group;

R₈₁ and R₈₂ are each independently selected from a hydrogen, a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF5, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₇-C₆₀ arylalkyl group, a substituted or unsubstituted C₁-C₆₀ heteroaryl group, a substituted or unsubstituted C₂-C₆₀ heteroaryloxy group, a substituted or unsubstituted C₂-C₆₀ heteroarylthio group, a substituted or unsubstituted C₃-C₆₀ heteroarylalkyl group, a substituted or unsubstituted monovalent non-aromatic condensed polycyclic group, a substituted or unsubstituted monovalent non-aromatic heterocondensed polycyclic group, —N(Q₁)(Q₂), —Si(Q₃)(Q₄)(Q₅), and —B(Q₆)(Q₇);

a81 and a82 are each independently an integer of 1 to 5;

n81 is an integer of 0 to 4;

n82 is 1, 2, or 3; and

L₈₁ is a monovalent organic ligand, a divalent organic ligand, or a trivalent organic ligand.

R₈₁ and R₈₂ may be understood by referring to the description provided herein in connection with R₁₁.

The phosphorescent dopant may include at least one selected from Compounds PD1 to PD78 and Flr₆, but embodiments are not limited thereto:

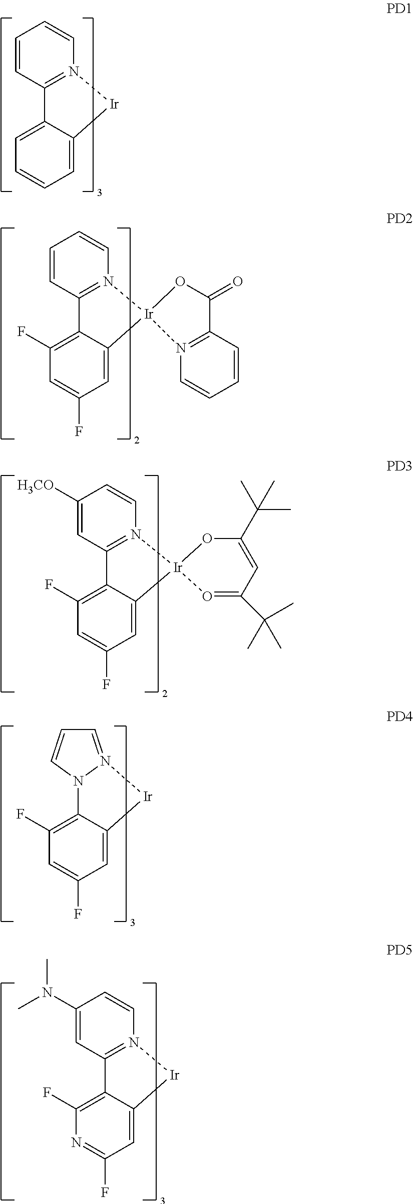

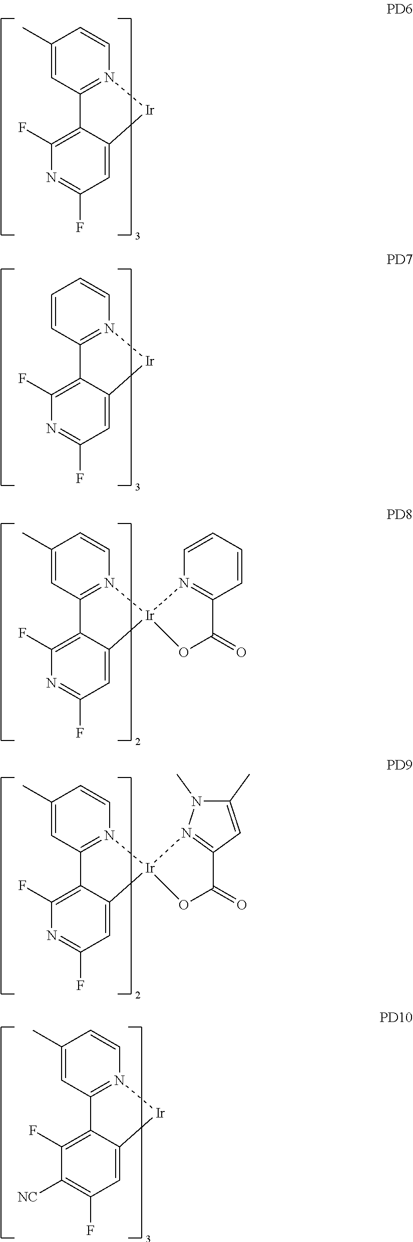

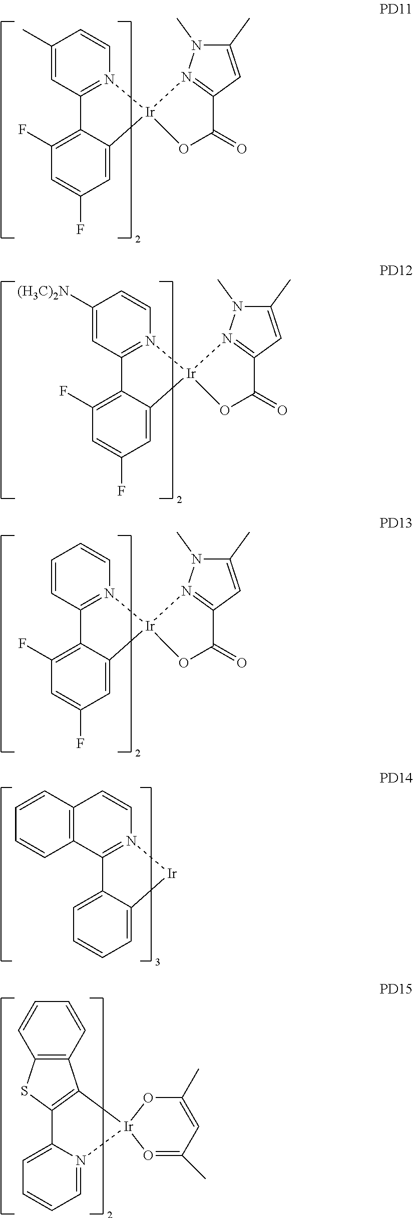

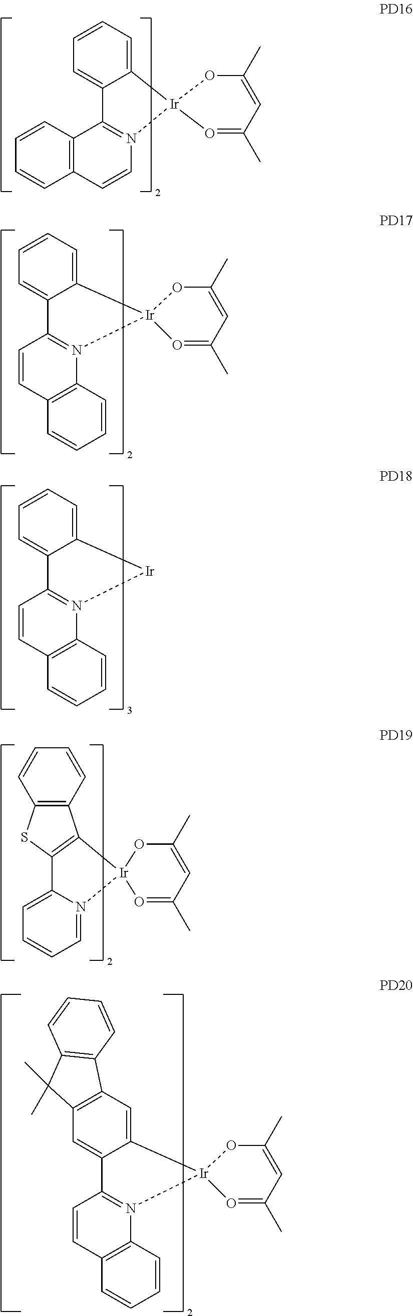

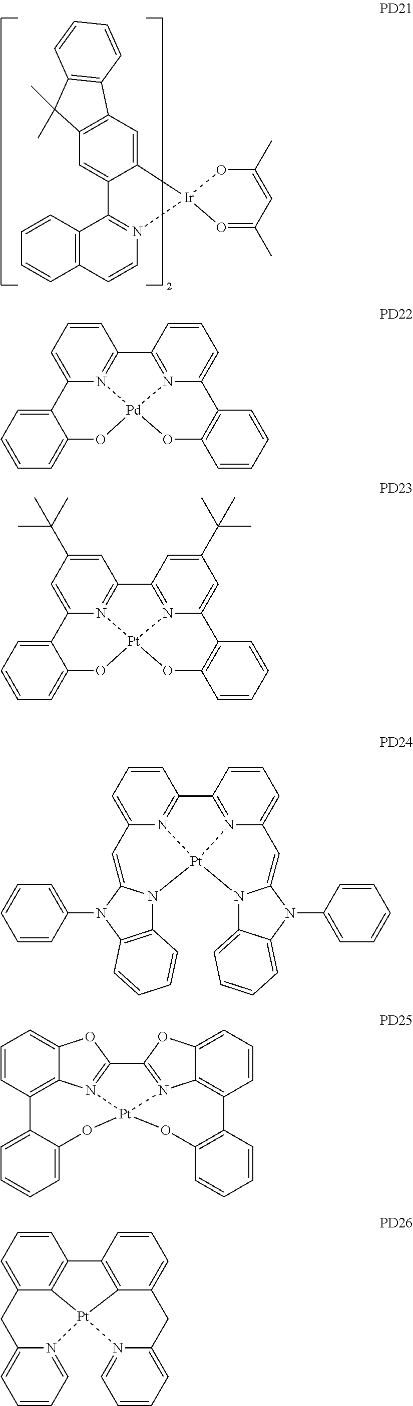

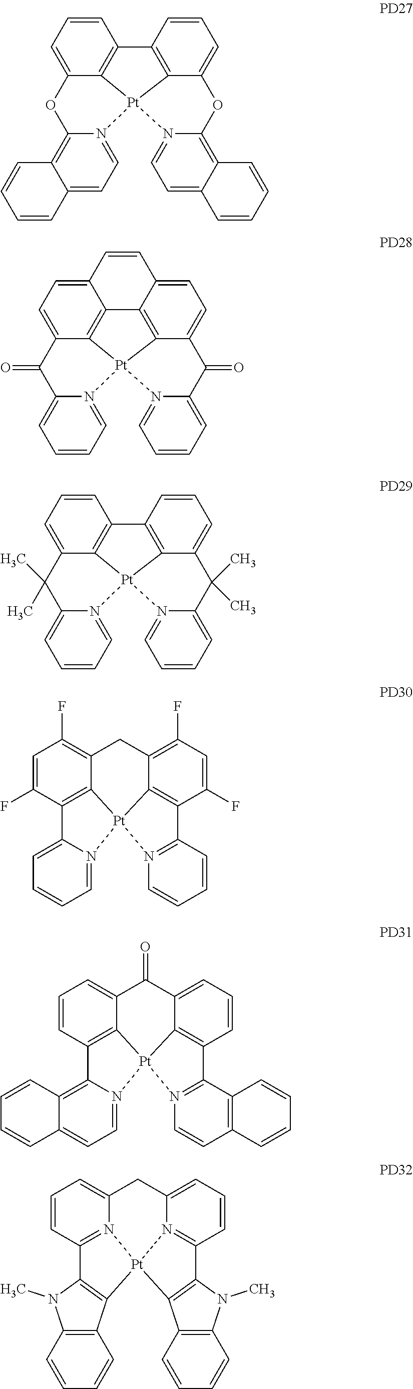

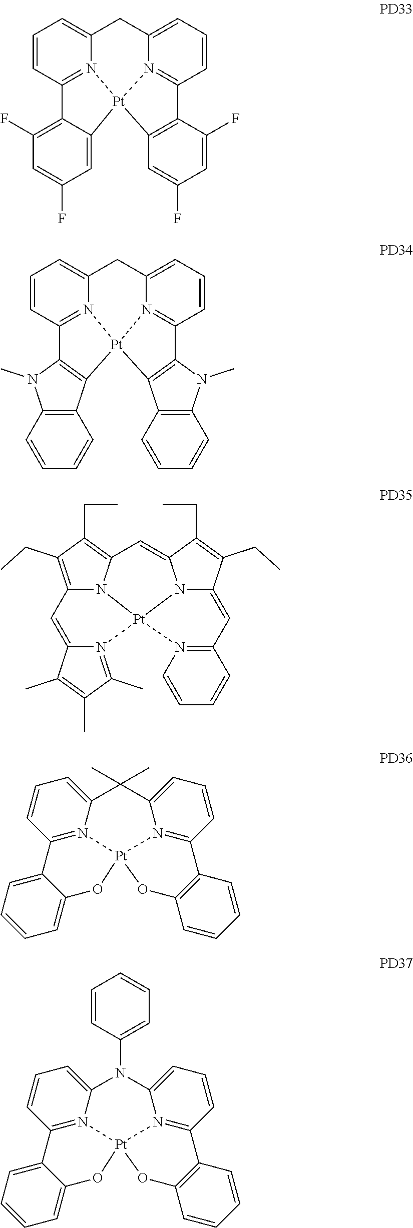

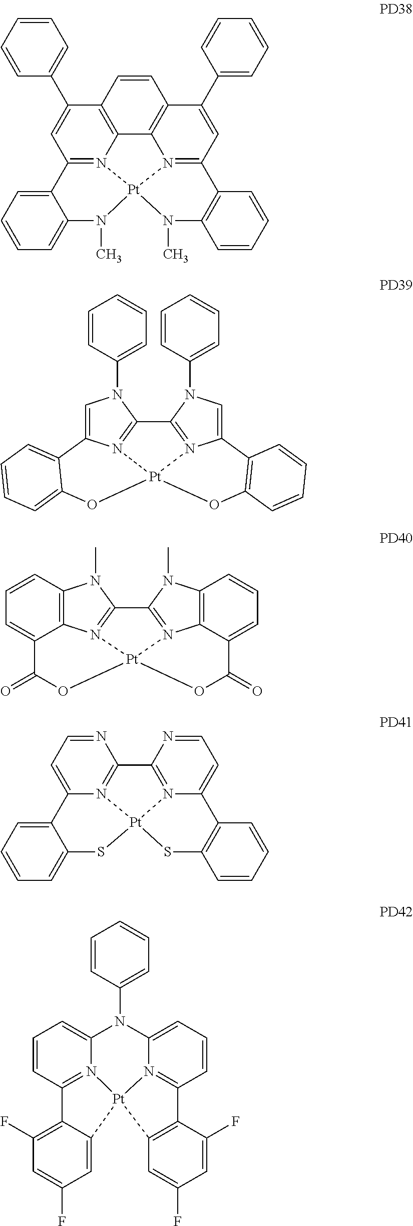

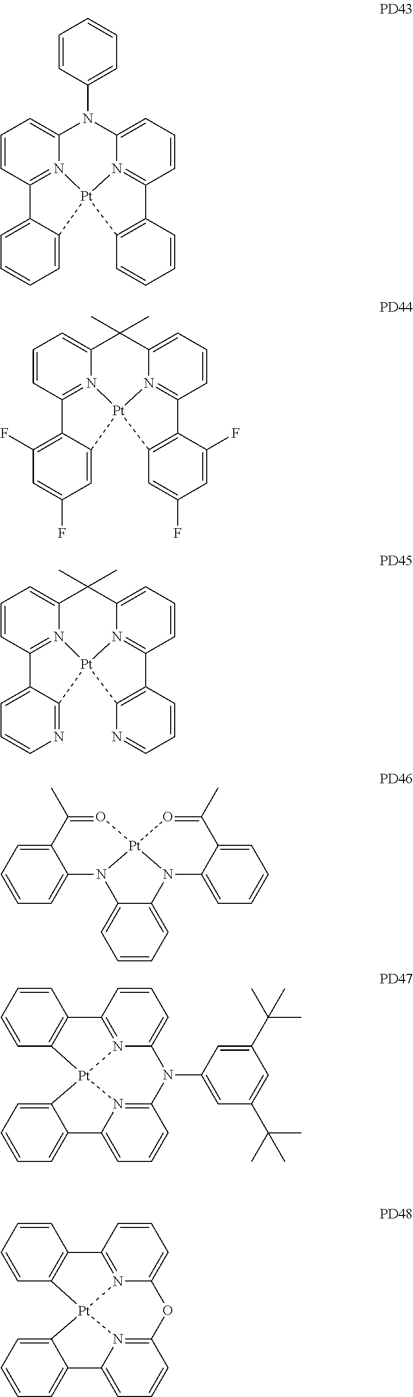

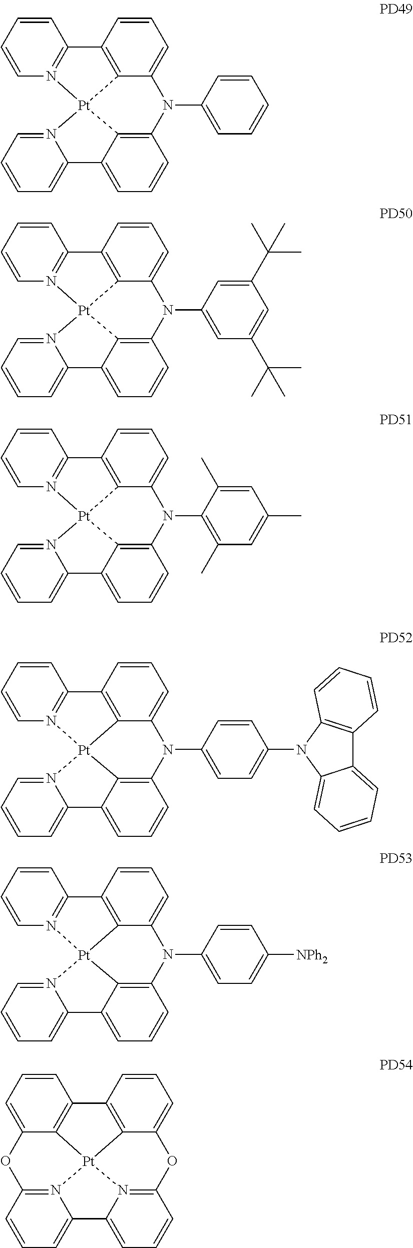

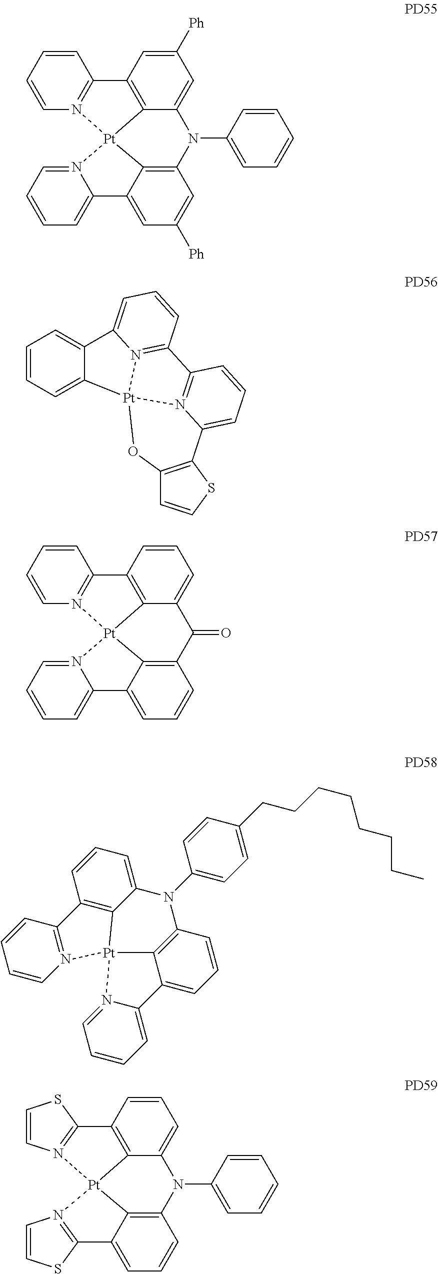

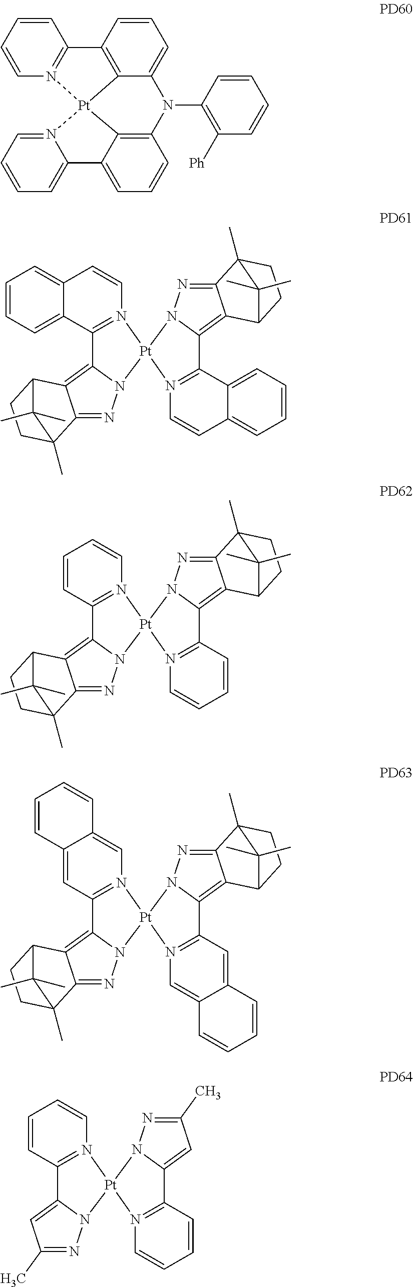

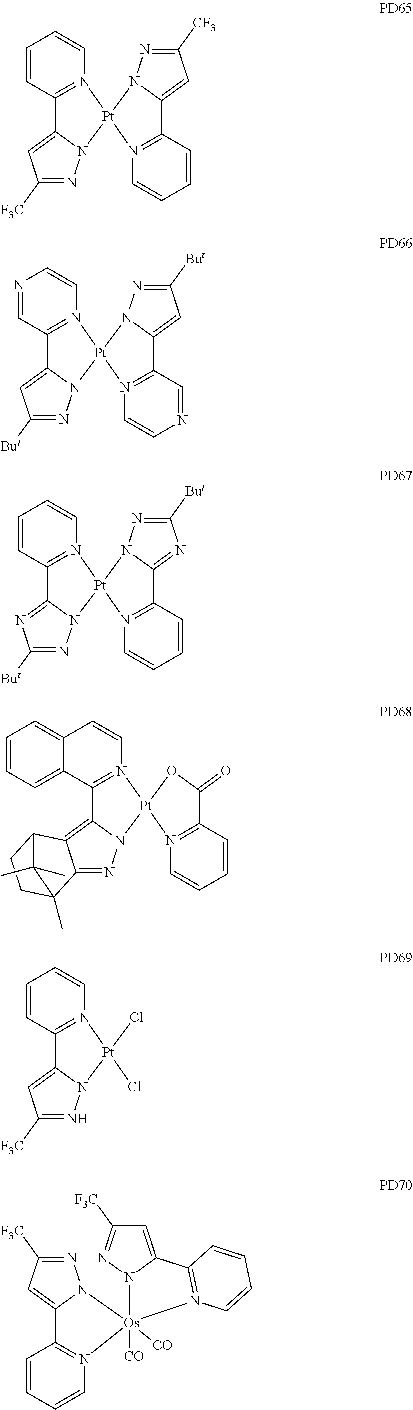

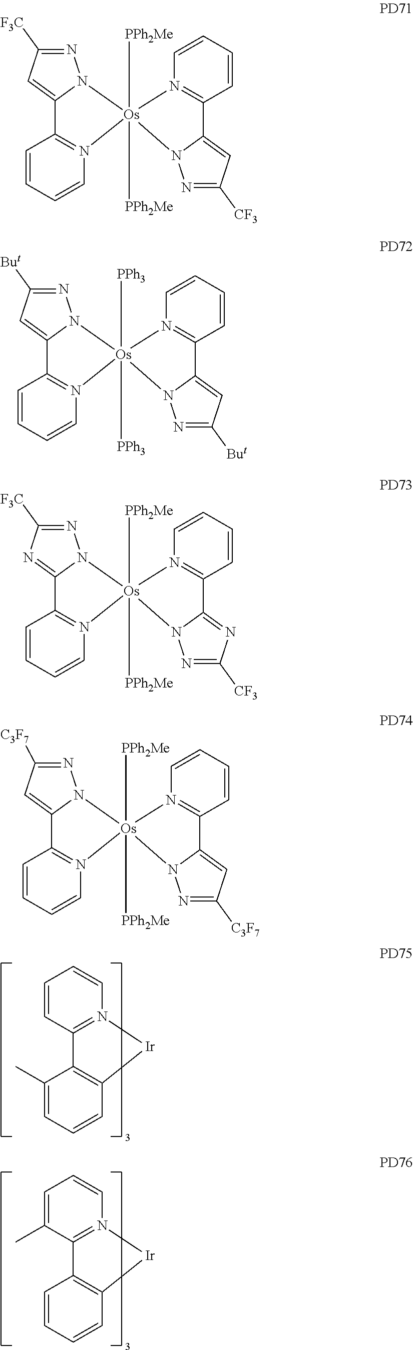

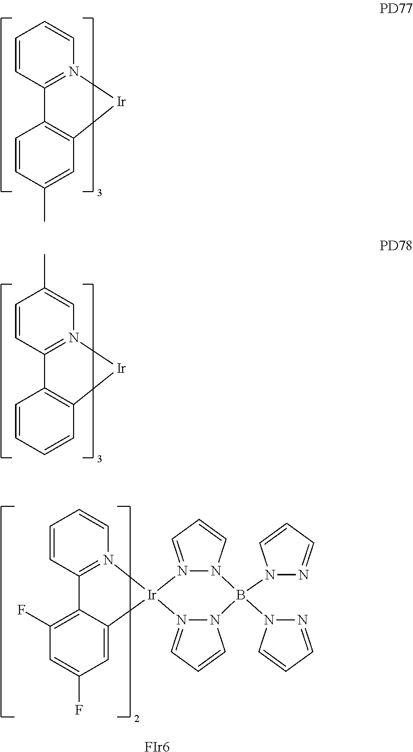

In some embodiments, the phosphorescent dopant may include PtOEP:

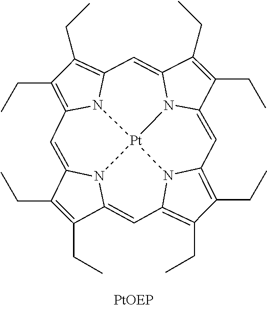

When the emission layer includes a host and a dopant, an amount of the dopant may be in a range of about 0.01 to about 20 parts by weight based on 100 parts by weight of the host, but is not limited thereto.

A thickness of the emission layer may be in a range of about 100 Å to about 1,000 Å, for example, about 200 Å to about 600 Å. When the thickness of the emission layer is within this range, excellent light-emission characteristics may be obtained without a substantial increase in driving voltage.

Then, an electron transport region may be disposed on the emission layer.

The electron transport region may include at least one selected from a hole blocking layer, an electron transport layer, and an electron injection layer.

For example, the electron transport region may have a structure of hole blocking layer/electron transport layer/electron injection layer or a structure of electron transport layer/electron injection layer, but the structure of the electron transport region is not limited thereto. The electron transport layer may have a single-layered structure or a multi-layer structure including two or more different materials.

Conditions for forming the hole blocking layer, the electron transport layer, and the electron injection layer which constitute the electron transport region may be understood by referring to the conditions for forming the hole injection layer.

When the electron transport layer includes a hole blocking layer, the hole blocking layer may include, for example, at least one selected from BCP and Bphen, but embodiments are not limited thereto.

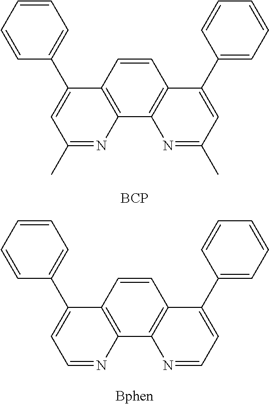

A thickness of the hole blocking layer may be in a range of about 20 Å to about 1,000 Å, for example, about 30 Å to about 300 Å. When the thickness of the hole blocking layer is within these ranges, the hole blocking layer may have improved hole blocking ability without a substantial increase in driving voltage.

The electron transport layer may further include at least one selected from BCP, Bphen, Alq₃, Balq, TAZ, and NTAZ.

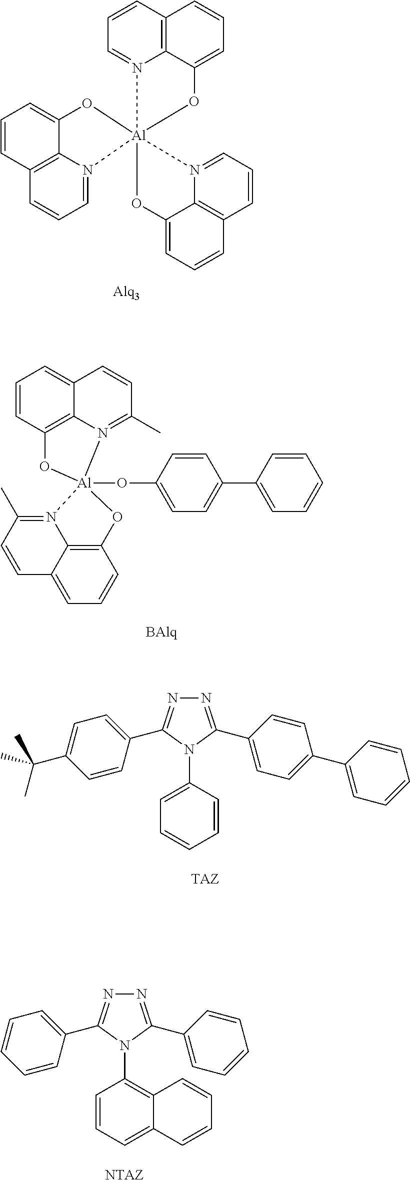

In some embodiments, the electron transport layer may include at least one selected from Compounds ET1, ET2, and ET3, but embodiments are not limited thereto:

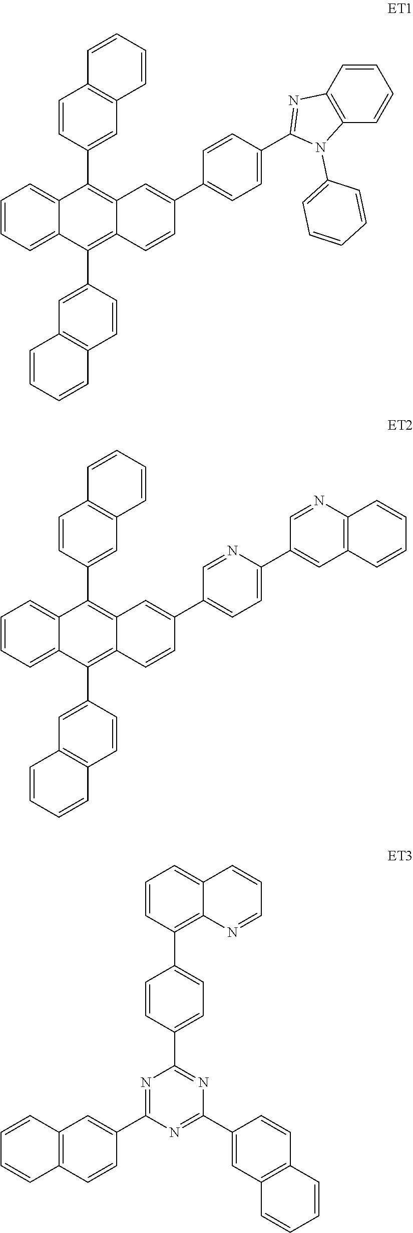 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc2cc(-c3nc(-c4ccc(-c5cccc6cccnc56)cc4)nc(-c4ccc5ccccc5c4)n3)ccc2c1]

A thickness of the electron transport layer may be in a range of about 100 Å to about 1,000 Å, for example, about 150 Å to about 500 Å. When the thickness of the electron transport layer is within the range described above, the electron transport layer may have satisfactory electron transport characteristics without a substantial increase in driving voltage.

Also, the electron transport layer may further include, in addition to the materials described above, a metal-containing material.

The metal-containing material may include a Li complex. The Li complex may include, for example, Compound ET-D1 (lithium quinolate, LiQ) or ET-D2.

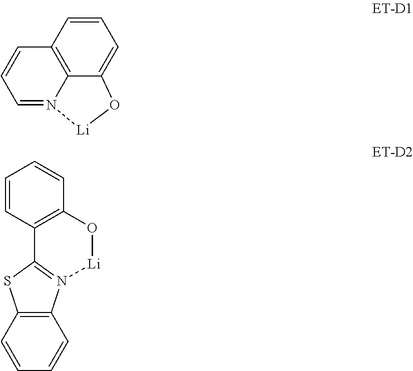

The electron transport layer may include an electron injection layer (EIL) that promotes flow of electrons from the second electrode 19 thereinto.

The electron injection layer may include at least one selected from, LiF, NaCl, CsF, Li₂O, BaO, and LiQ.

A thickness of the electron injection layer may be in a range of about 1 Å to about 100 Å, about 3 Å to about 90 Å. When the thickness of the electron injection layer is within the range described above, the electron injection layer may have satisfactory electron injection characteristics without a substantial increase in driving voltage.

The second electrode 19 is disposed on the organic layer 15. The second electrode 19 may be a cathode. A material for forming the second electrode 19 may be selected from metal, an alloy, an electrically conductive compound, and a combination thereof, which have a relatively low work function. For example, lithium (Li), magnesium (Mg), aluminum (Al), aluminum-lithium (Al—Li), calcium (Ca), magnesium-indium (Mg—In), or magnesium-silver (Mg—Ag) may be formed as a material for forming the second electrode 19. In some embodiments, to manufacture a top emission type light-emitting device, a transmissive electrode formed using ITO or IZO may be used as the second electrode 19.

Hereinbefore, the organic light-emitting device has been described with reference to FIG. 1, but is not limited thereto.

A C₁-C₆₀ alkyl group as used herein refers to a linear or branched aliphatic hydrocarbon monovalent group having 1 to 60 carbon atoms. Detailed examples thereof are a methyl group, an ethyl group, a propyl group, an isobutyl group, a sec-butyl group, a tert-butyl group, a pentyl group, an iso-amyl group, and a hexyl group. A C₁-C₆₀ alkylene group as used herein refers to a divalent group having the same structure as the C₁-C₆₀ alkyl group.

A C₁-C₆₀ alkoxy group as used herein refers to a monovalent group represented by —OA₁₀₁ (wherein A₁₀₁ is the C₁-C₆₀ alkyl group). Detailed examples thereof are a methoxy group, an ethoxy group, and an isopropyloxy group.

A C₂-C₆₀ alkenyl group as used herein refers to a hydrocarbon group formed by substituting at least one carbon double bond in the middle or at the terminal of the C₂-C₆₀ alkyl group. Detailed examples thereof are an ethenyl group, a propenyl group, and a butenyl group. A C₂-C₆₀ alkenylene group as used herein refers to a divalent group having the same structure as the C₂-C₆₀ alkenyl group.

A C₂-C₆₀ alkynyl group as used herein refers to a hydrocarbon group formed by substituting at least one carbon trip bond in the middle or at the terminal of the C₂-C₆₀ alkyl group. Detailed examples thereof are an ethynyl group, and a propynyl group. A C₂-C₆₀ alkynylene group as used herein refers to a divalent group having the same structure as the C₂-C₆₀ alkynyl group.

A C₃-C₁₀ cycloalkyl group as used herein refers to a monovalent hydrocarbon monocyclic group having 3 to 10 carbon atoms. Detailed examples thereof are a cyclopropyl group, a cyclobutyl group, a cyclopentyl group, a cyclohexyl group, and a cycloheptyl group. A C₃-C₁₀ cycloalkylene group as used herein refers to a divalent group having the same structure as the C₃-C₁₀ cycloalkyl group.

A C₁-C₁₀ heterocycloalkyl group as used herein refers to a monovalent monocyclic group having at least one hetero atom selected from N, O, P, and S as a ring-forming atom and 1 to 10 carbon atoms. Detailed examples thereof are a tetrahydrofuranyl group, and a tetrahydrothiophenyl group. A C₁-C₁₀ heterocycloalkylene group as used herein refers to a divalent group having the same structure as the C₁-C_{1c}, heterocycloalkyl group.

A C₃-C₁₀ cycloalkenyl group as used herein refers to a monovalent monocyclic group that has 3 to 10 carbon atoms and at least one double bond in the ring thereof and which is not aromatic. Detailed examples thereof are a cyclopentenyl group, a cyclohexenyl group, and a cycloheptenyl group. A C₃-C₁₀ cycloalkenylene group as used herein refers to a divalent group having the same structure as the C₃-C₁₀ cycloalkenyl group.

A C₁-C₁₀ heterocycloalkenyl group as used herein refers to a monovalent monocyclic group that has at least one hetero atom selected from N, O, P, and S as a ring-forming atom, 1 to 10 carbon atoms, and at least one double bond in its ring. Detailed examples of the C₂-C₁₀ heterocycloalkenyl group are a 2,3-dihydrofuranyl group and a 2,3-dihydrothiophenyl group. A C₁-C₁₀ heterocycloalkenylene group as used herein refers to a divalent group having the same structure as the C₁-C₁₀ heterocycloalkenyl group.

A C₆-C₆₀ aryl group as used herein refers to a monovalent group having a carbocyclic aromatic system having 6 to 60 carbon atoms, and a C₆-C₆₀ arylene group as used herein refers to a divalent group having a carbocyclic aromatic system having 6 to 60 carbon atoms. Detailed examples of the C₆-C₆₀ aryl group are a phenyl group, a naphthyl group, an anthracenyl group, a phenanthrenyl group, a pyrenyl group, and a chrysenyl group. When the C₆-C₆₀ aryl group and the C₆-C₆₀ arylene group each include two or more rings, the rings may be fused to each other.

A C₁-C₆₀ heteroaryl group as used herein refers to a monovalent group having a carbocyclic aromatic system that has at least one hetero atom selected from N, O, P, and S as a ring-forming atom, and 1 to 60 carbon atoms. A C₁-C₆₀ heteroarylene group as used herein refers to a divalent group having a carbocyclic aromatic system that has at least one hetero atom selected from N, O, P, and S as a ring-forming atom, and 1 to 60 carbon atoms. Examples of the C₁-C₆₀ heteroaryl group are a pyridinyl group, a pyrimidinyl group, a pyrazinyl group, a pyridazinyl group, a triazinyl group, a quinolinyl group, and an isoquinolinyl group. When the C₁-C₆₀ heteroaryl group and the C₁-C₆₀ heteroarylene group each include two or more rings, the rings may be fused to each other.

A C₆-C₆₀ aryloxy group as used herein indicates —OA₁₀₂ (wherein A₁₀₂ is the C₆-C₆₀ aryl group), a C₆-C₆₀ arylthio group as used herein indicates —SA₁₀₃ (wherein A₁₀₃ is the C₆-C₆₀ aryl group), and a C₇-C₆₀ arylalkyl as used herein indicates A₁₀₄A₁₀₅ (wherein A₁₀₅ is the C₆-C₆₀ aryl group and A₁₀₄ is the C₁-C₆₀ alkyl group).

A C₂-C₆₀ heteroaryloxy as used herein indicates —OA₁₀₆ (wherein A₁₀₆ is the C₂-C₆₀ heteroaryl group), a C₂-C₆₀ heteroarylthio indicates —SA₁₀₇ (wherein A₁₀₇ is the C₂-C₆₀ heteroaryl group), and a C₃-C₆₀ heteroarylalkyl indicates -A₁₀₈A₁₀₀ (wherein A₁₀₉ is the C₂-C₆₀ heteroaryl group and A₁₀₈ is the C₁-C₆₀ alkyl group).

A monovalent non-aromatic condensed polycyclic group as used herein refers to a monovalent group that has two or more rings condensed to each other, only carbon atoms (for example, the number of carbon atoms may be in a range of 8 to 60) as a ring forming atom, and which is non-aromatic in the entire molecular structure. An example of the monovalent non-aromatic condensed polycyclic group is a fluorenyl group. A divalent non-aromatic condensed polycyclic group as used herein refers to a divalent group having the same structure as the monovalent non-aromatic condensed polycyclic group.

A monovalent non-aromatic condensed heteropolycyclic group as used herein refers to a monovalent group that has two or more rings condensed to each other, has a heteroatom selected from N, O P, and S, other than carbon atoms (for example, the number of carbon atoms may be in a range of 1 to 60), as a ring forming atom, and which is non-aromatic in the entire molecular structure. An example of the monovalent non-aromatic condensed heteropolycyclic group is a carbazolyl group. A divalent non-aromatic condensed heteropolycyclic group as used herein refers to a divalent group having the same structure as the monovalent non-aromatic condensed heteropolycyclic group.

At least one of substituents of the substituted C₃-C₁₀ cycloalkylene group, substituted C₁-C₁₀ heterocycloalkylene group, substituted C₃-C₁₀ cycloalkenylene group, substituted C₁-C₁₀ heterocycloalkenylene group, substituted C₆-C₆₀ arylene group, substituted C₁-C₆₀ heteroarylene group, substituted a divalent non-aromatic condensed polycyclic group, substituted a divalent non-aromatic condensed heteropolycyclic group, substituted C₁-C₆₀ alkyl group, substituted C₂-C₆₀ alkenyl group, substituted C₂-C₆₀ alkynyl group, substituted C₁-C₆₀ alkoxy group, substituted C₃-C₁₀ cycloalkyl group, substituted C₁-C₁₀ heterocycloalkyl group, substituted C₃-C₁₀ cycloalkenyl group, substituted C₁-C₁₀ heterocycloalkenyl group, substituted C₆-C₆₀ aryl group, substituted C₆-C₆₀ aryloxy group, substituted C₆-C₆₀ arylthio group, substituted C₇-C₆₀ arylalkyl group, substituted C₁-C₆₀ heteroaryl group, substituted C₂-C₆₀ heteroaryloxy group, substituted C₂-C₆₀ heteroarylthio group, substituted C₃-C₆₀ heteroarylalkyl group, substituted monovalent non-aromatic condensed polycyclic group, and substituted monovalent non-aromatic condensed heteropolycyclic group as used herein may be selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, a monovalent non-aromatic condensed heteropolycyclic group (provided that the monovalent non-aromatic condensed heteropolycyclic group is not a carbazolyl group) and —Si(Q₃₁)(Q₃₂)(Q₃₃),

wherein Q₁ to Q₃, Q₁₁ to Q₁₃, Q₂₁ to Q₂₃, and Q₃₁ to Q₃₃ may be each independently selected from a hydrogen, a C₁-C₆₀ alkyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group (provided that the monovalent non-aromatic condensed heteropolycyclic group is not a carbazolyl group).

The “biphenyl group” as used herein refers to “a phenyl group substituted with a phenyl group.”

Hereinafter, a compound and an organic light-emitting device according to embodiments are described in detail with reference to Synthesis Example and Examples. However, the organic light-emitting device is not limited thereto. The wording “B was used instead of A” used in describing Synthesis Examples means that an amount of A used was identical to an amount of B used, in terms of a molar equivalent.

### EXAMPLE

### Synthesis Example 1

### Synthesis of Compound 28

10 grams (g) (52 millimoles (mmol)) of 3-cyanocarbazole, 14.2 g of 2,8-dibromodibenzo[b,d]furan, 8.8 g of CuI, 15.9 g of K₂CO₃, and 2.5 g of 1,10-phenanthroline were placed in a 250 milliliter (mL) 2-neck round-bottom flask, and 150 mL of DMF was added thereto. The mixture was stirred at a temperature of 150° C. for 28 hours. The reaction product was cooled and MeOH was added thereto to produce a solid, which was then filtered therefrom. The resulting solid was mixed with 1 liter (L) of chloroform, dissolved by heating, and filtered through a celite pad. The residual solution obtained therefrom was concentrated under reduced pressure. The resulting solid was recrystallized with a mixture of methylene chloride (MC)/Acetone to produce Compound 28 (12.3 g, yield of 43%).

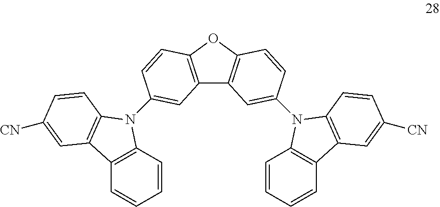

Calc.: 548.16. found [M+H⁺]: 549.16.

### Synthesis Example 2

### Synthesis of Compound 4

Compound 4 (10.3 g, yield of 61.6%) was prepared in the same manner as in Synthesis Example 1, except that 2,6-dichloropyridine was used instead of 2,8-dibromodibenzo[b,d]furan.

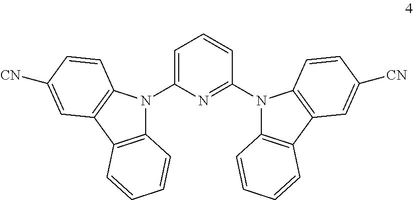

Calc.: 459.15. found [M+H⁺]: 460.15.

### Synthesis Example 3

### Synthesis of Compound 3

Compound 3 (10.9 g, yield of 65.3%) was prepared in the same manner as in Synthesis Example 1, except that 3,6-dicyanocarbazole was used instead of 3-cyanocarbazole, and 9-(3-bromophenyl)-9H-carbazole was used instead of 2,8-dibromodibenzo[b,d]furan.

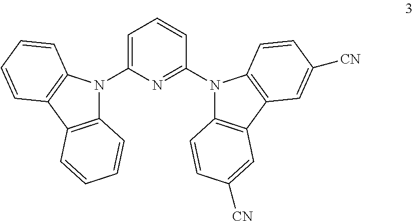 [SMILES: N#Cc1ccc2c(c1)c1cc(C#N)ccc1n2-c1cccc(-n2c3ccccc3c3ccccc32)n1]

Calc.: 458.15. found [M+H⁺]: 459.16.

### Synthesis Example 4

### Synthesis of Compound 6

Compound 6 (13.2 g, yield of 53.6%) was prepared in the same manner as in Synthesis Example 1, except that 3,3′-dibromo-1,1′-biphenyl was used instead of 2,8-dibromodibenzo[b,d]furan.

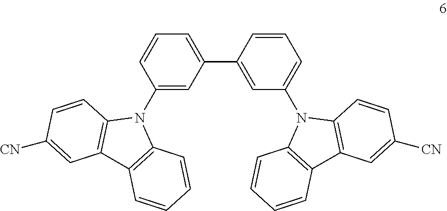

Calc.: 534.18. found [M+H⁺]: 535.18.

### Synthesis Example 5

### Synthesis of Compound 29

Compound 29 (11.7 g, yield of 64.4%) was prepared in the same manner as in Synthesis Example 1, except that 3,6-dicyanocarbazole was used instead of 3-cyanocarbazole, and 9-(8-bromodibenzo[b,d]furan-2-yl)-9H-carbazole) was used instead of 2,8-dibromodibenzo[b,d]furan.

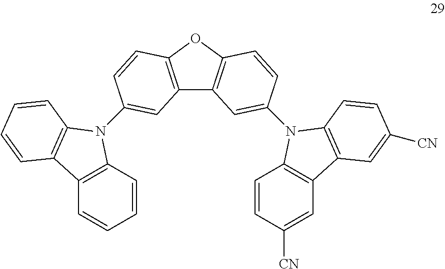 [SMILES: N#Cc1ccc2c(c1)c1cc(C#N)ccc1n2-c1ccc2oc3ccc(-n4c5ccccc5c5ccccc54)cc3c2c1]

Calc.: 548.16. found [M+H⁺]: 549.16.

### Synthesis Example 6

### Synthesis of Compound 1

Compound 1 (12.5 g, yield of 60.8%) was prepared in the same manner as in Synthesis Example 1, except that 3,6-dicyanocarbazole was used instead of 3-cyanocarbazole, and 9-(3′-bromo-[1,1′-biphenyl]-3-yl)-9H-carbazole was used instead of 2,8-dibromodibenzo[b,d]furan.

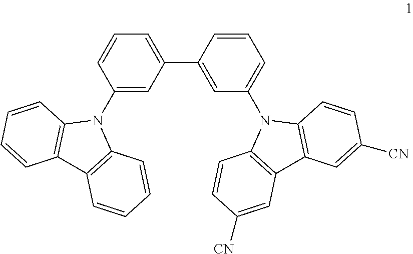

Calc.: 534.18. found [M+H⁺]: 535.17.

### Synthesis Example 7

### Synthesis of Compound 5

Compound 5 (9.8 g, yield of 58.6%) was prepared in the same manner as in Synthesis Example 1, except that 3,6-dicyanocarbazole was used instead of 3-cyanocarbazole, and 9-(6-chloropyridin-2-yl)-9H-carbazole was used instead of 2,8-dibromodibenzo[b,d]furan.

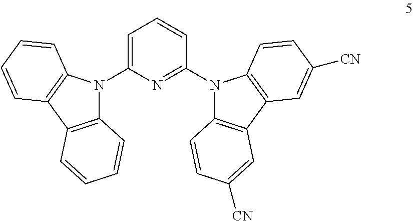 [SMILES: N#Cc1ccc2c(c1)c1cc(C#N)ccc1n2-c1cccc(-n2c3ccccc3c3ccccc32)n1]

Calc.: 459.15. found [M+H⁺]: 460.16.

### Synthesis Example 8

### Synthesis of Compound 2

Compound 2 (10.5 g, yield of 57.8%) was prepared in the same manner as in Synthesis Example 1, except that 1,3-dichlorobenzene was used instead of 2,8-dibromodibenzo[b,d]furan.

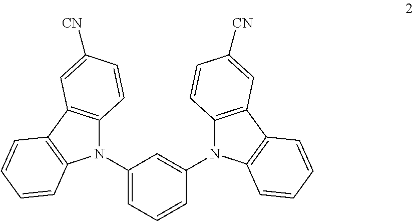

Calc.: 458.15. found [M+H⁺]: 459.16.

### Synthesis Example 9

### Synthesis of Compound 24

Compound 24 (10.3 g, yield of 73.4%)_was prepared in the same manner as in Synthesis Example 1, except that 2,8-dibromodibenzo[b,d]thiophene was used instead of 2,8-dibromodibenzo[b,d]furan.

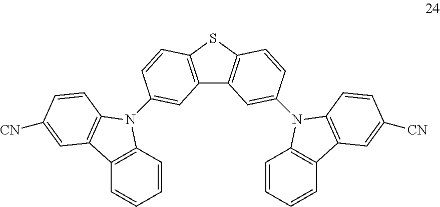

Calc.: 564.14. found [M+H⁺]: 565.14.

### Evaluation Example 1

### Evaluation of HOMO, LUMO and Triplet (T1) Energy Level

HOMO, LUMO and T₁ energy levels of Compounds 4, 28, 6, 5, 3 and 2 were evaluated by using methods shown in Table 2. Results thereof are shown in Table 3.

TABLE 2

| | |
|---|---|
| HOMO energy level | A potential (Volts, V)-current (Amperes, A) graph |
| evaluation method | of each compound was obtained by using cyclic |
| | voltammetry (CV) (electrolyte: 0.1 M Bu₄NClO₄/ |
| | solvent: CH₂Cl₂/electrode: 3 electrode system |
| | (working electrode: GC, reference electrode: |
| | Ag/AgCl, auxiliary electrode: Pt)). Then, from |
| | reduction onset of the graph, a HOMO energy |
| | level of the compound was calculated. |
| LUMO energy level | Each compound was diluted at a concentration of |
| evaluation method | 1 × 10⁻⁵ molar (M) in CHCl₃, and an UV |
| | absorption spectrum thereof was measured at room |
| | temperature by using a Shimadzu UV-350 |
| | spectrometer, and a LUMO energy level thereof |
| | was calculated by using an optical band gap (Eg) |
| | from an edge of the absorption spectrum. |
| T1 energy level | A mixture (each compound was dissolved in an |
| evaluation method | amount of 1 milligrams (mg) in 3 cubic |
| | centimeters (cc) of toluene) of toluene and each |
| | of the compounds was loaded into a quartz cell. |
| | The resultant quartz cell was loaded into liquid |
| | nitrogen (77 Kelvin (K)) and a photoluminescence |
| | spectrum thereof was measured by using a device |
| | for measuring photoluminescence. The obtained |
| | spectrum was compared with a photoluminescence |
| | spectrum measured at room temperature, and peaks |
| | observed only at low temperature were analyzed |
| | to calculate T₁ energy levels. |

TABLE 3

| | HOMO (eV) | LUMO (eV) | T₁ energy |
|---|---|---|---|
| Compound No. | (found) | (found) | level (eV) |
| Compound 4 | −6.00 | −2.43 | 3.04 |
| Compound 28 | −5.77 | −2.28 | 3.02 |
| Compound 6 | −5.89 | −2.36 | 2.82 |
| Compound 5 | −5.74 | −2.21 | 3.02 |
| Compound 3 | −5.71 | −2.15 | 3.02 |
| Compound 2 | −5.91 | −2.36 | 3.04 |

From Table 3, it may be seen that Compounds 4, 28, 6, 5, 3, and 2 are suitable for use as a material for an organic light-emitting device.

### Evaluation Example 2

### Thermal Characteristics Evaluation

Each of Compounds 4, 28, 6, 5, 3, 2, and A was subjected to thermal analysis (N₂ atmosphere, temperature range: room temperature to 800° C. (10° C./min)-TGA, room temperature to 400° C.-DSC, Pan Type: Pt Pan in disposable Al Pan(TGA), disposable Al pan(DSC)) using thermo gravimetric analysis (TGA) and differential scanning calorimetry (DSC), and obtained results are shown in Table 4 below. Referring to Table 4, it may be seen that Compounds 4, 28, 6, 5, 3, and 2 may have higher thermal stability than Compound A.

TABLE 4

| | Tg |
|---|---|
| Compound No. | (° C.) |
| 4 | 126 |
| 28 | 174 |
| 6 | 134 |
| 5 | 130 |
| 2 | 121 |
| 3 | 130 |
| Compound A | 72 |

Compound A

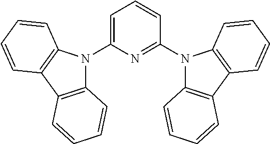 [SMILES: c1cc(-n2c3ccccc3c3ccccc32)nc(-n2c3ccccc3c3ccccc32)c1]

### Evaluation Example 3

### Heat Resistance of Thin Film

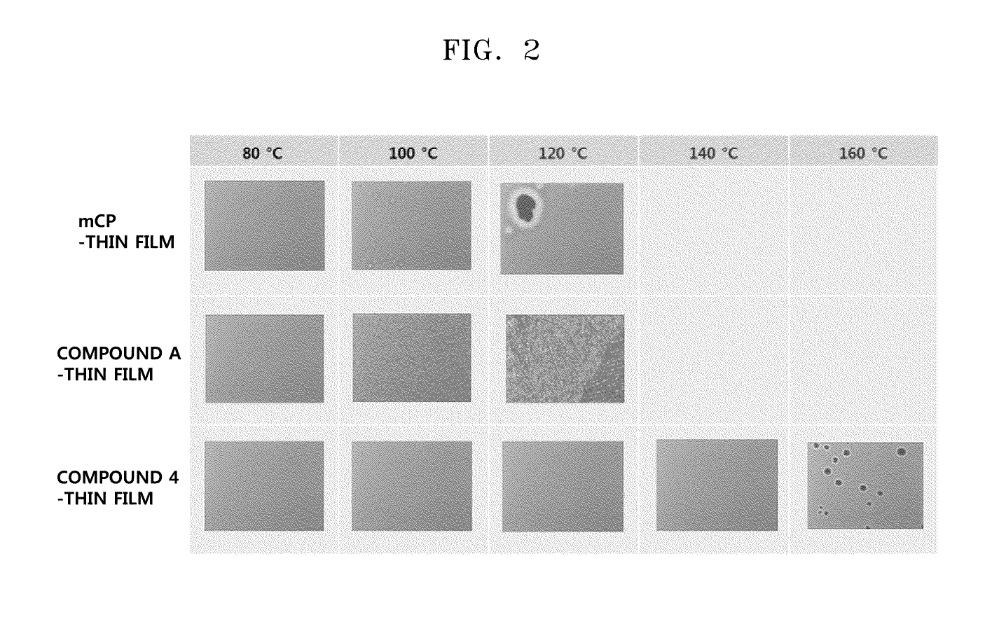

Each of mCP, Compound A, and Compound 4 was vacuum deposited on a glass substrate to form an mCP-thin film having a thickness of 500 Å, a Compound A-thin film having a thickness of 500 Å, and a Compound 4-thin film having a thickness of 500 Å. The mCP-thin film, the Compound A-thin film, and the Compound 4-thin film on the glass substrate were mounted on a hot-plate under ambient condition (relative humidity of 50%) at a temperature of 80° C., and heated at incremental rates of 20° C. every 10 minutes. In each step, images of surfaces of these thin films were captured, and FIG. 2 shows the captured images thereof.

Referring to FIG. 2, it may be seen that when the temperature of the hot-plate is 120° C., the surface of the mCP-thin film has black spots, and the surface of the Compound A-thin film is crystallized. However, in the case of the Compound 4-thin film, even at a temperature of 140° C., black spots and crystallization did not occur.

From these results, it may be seen that the Compound 4-thin film has excellent thin film heat resistance.

### Example 1

A glass substrate with a 1,500 Å-thick ITO (Indium tin oxide) electrode (first electrode, anode) formed thereon was washed with distilled water and ultrasonic waves. When the washing with distilled water was completed, sonification washing was performed using a solvent, such as isopropyl alcohol, acetone, or methanol. The result was dried and then transferred to a plasma washer. The resultant substrate was washed with oxygen plasma for 5 minutes and transferred to a vacuum depositing device.

Compound HT3 and Compound HP-1 were co-deposited on the ITO electrode on the glass substrate to form a hole injection layer having a thickness of 100 Å. Then, Compound HT3 was deposited on the hole injection layer to form a hole transport layer having a thickness of 1,300 Å, and mCP was deposited on the hole transport layer to form an electron blocking layer having a thickness of 150 Å, thereby completing the manufacture of a hole transport region.

Compound 4 (host) and FIr6 (dopant, 10 percent by weight (wt %)) were co-deposited on the hole transport region to form an emission layer having a thickness of 300 Å.

BCP was vacuum deposited on the emission layer to form a hole blocking layer having a thickness of 100 Å, Compound ET3 and Liq were vacuum deposited on the hole blocking layer to form an electron transport layer having a thickness of 250 Å. Then, Liq was deposited on the electron transport layer to form an electron injection layer having a thickness of 5 Å, and Al second electrode(cathode) having a thickness of 1,000 Å was formed on the electron injection layer, thereby completing the manufacture of an organic light-emitting device.

### Examples 2 to 4 and Comparative Examples 1 to 5

Organic light-emitting devices were manufactured in the same manner as in Example 1, except that in forming an emission layer, as a host, the compounds shown in Table 5 were used instead of Compound 4.

### Evaluation Example 4

### Evaluation on Characteristics of Organic Light-Emitting Devices

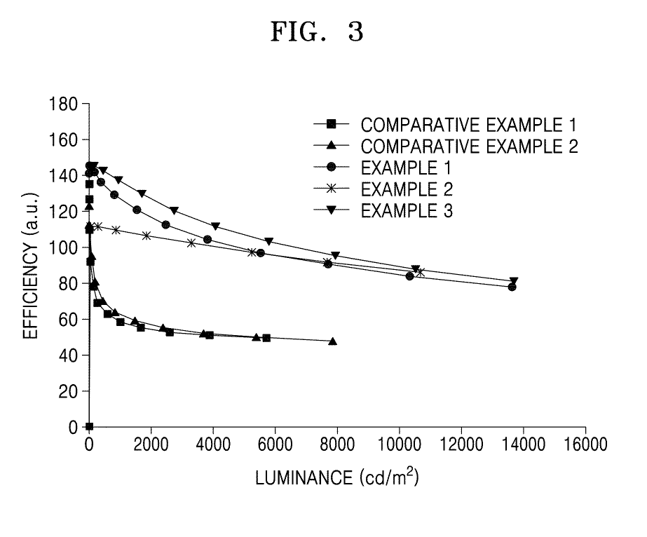

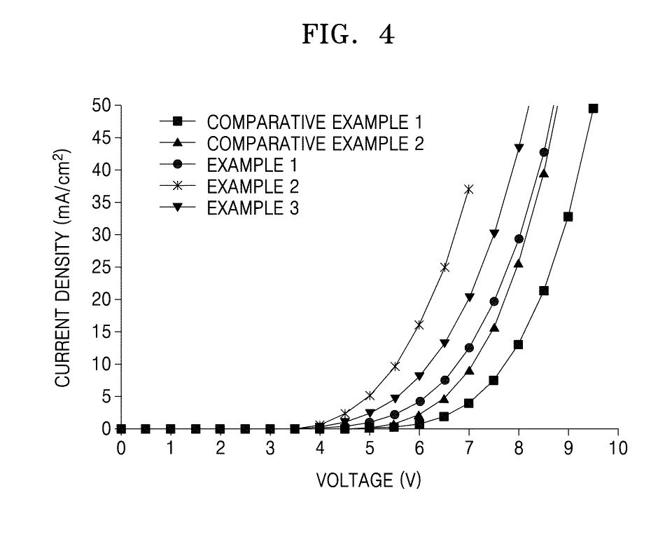

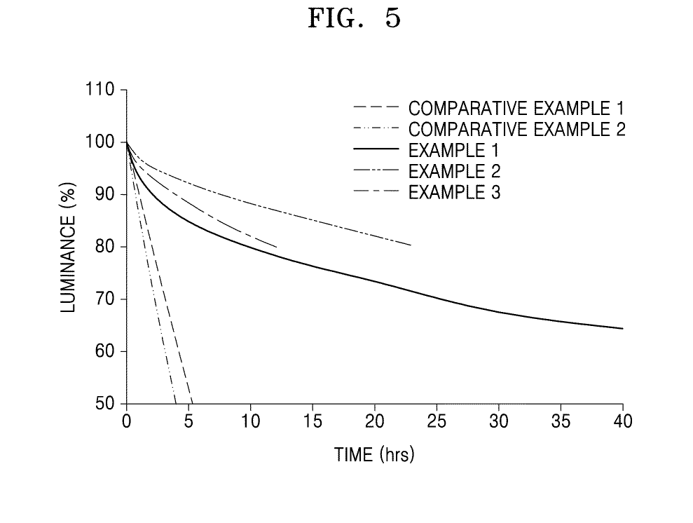

The driving voltage, current density, efficiency, power efficiency, quantum efficiency, and lifespan of the organic light-emitting devices of Examples 1 to 3 and Comparative Examples 1 to 5 were measured by using a current-voltage meter (Keithley 2400) and a luminance meter (Minolta Cs-1000A), and results thereof are shown in Table 5. FIGS. 3, 4, and 5 show a luminance-efficiency graph, a voltage-current density graph, and a time-luminance graph of the organic light-emitting devices of Examples 1 to 3 and Comparative Examples 1 and 2, respectively.

T₉₅ (at 500 candelas per square meter (cd/m²)) in Table 5 indicates an amount of time that lapsed when 100% of the initial luminance decreased to 95%.

TABLE 5

| | | Driving | Current | | | Quantum | |
|---|---|---|---|---|---|---|---|
| | | Voltage | density | Efficiency | Power | efficiency | T₉₅ |
| | Host | (V) | (mA/cm²) | (cd/A) | (Im/W) | (%) | (hr) |
| Example 1 | Compound 4 | 5.12 | 1.29 | 39.25 | 24.16 | 19.5 | 0.7 |
| Example 2 | Compound 28 | 4.19 | 1.36 | 36.96 | 27.80 | 17.0 | 2.2 |
| Example 3 | Compound 6 | 4.55 | 1.27 | 39.43 | 27.26 | 20.3 | 1.3 |
| Example 4 | Compound 24 | 4.53 | 1.25 | 39.73 | 27.5 | 21.1 | 1.96 |
| Comparative | Compound A | 6.85 | 3.32 | 15.27 | 7.03 | 8.9 | 0.5 |
| Example 1 | | | | | | | |
| Comparative | Compound B | 6.09 | 2.64 | 19.20 | 9.93 | 10.2 | 0.4 |
| Example 2 | | | | | | | |
| Comparative | Compound C | 5.22 | 1.89 | 13.83 | 8.32 | 7.4 | 0.2 |
| Example 3 | | | | | | | |
| Comparative | Compound D | 5.02 | 2.45 | 11.44 | 7.16 | 6.1 | 0.3 |
| Example 4 | | | | | | | |
| Comparative | Compound E | 5.92 | 1.29 | 22.51 | 11.90 | 12.0 | 0.2 |
| Example 5 | | | | | | | |

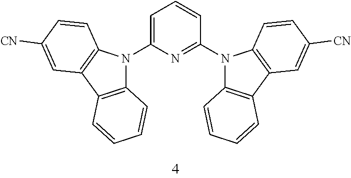

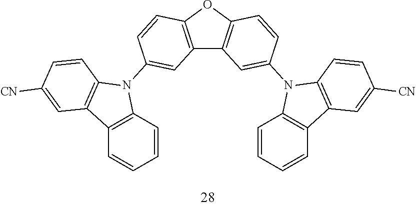

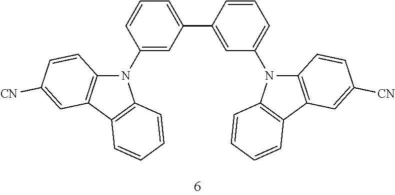

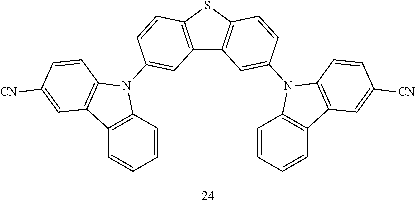

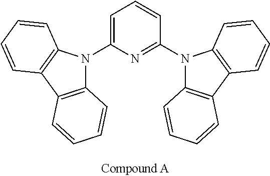 [SMILES: c1cc(-n2c3ccccc3c3ccccc32)nc(-n2c3ccccc3c3ccccc32)c1]

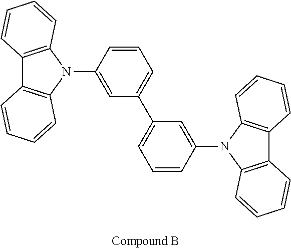 [SMILES: c1cc(-c2cccc(-n3c4ccccc4c4ccccc43)c2)cc(-n2c3ccccc3c3ccccc32)c1]

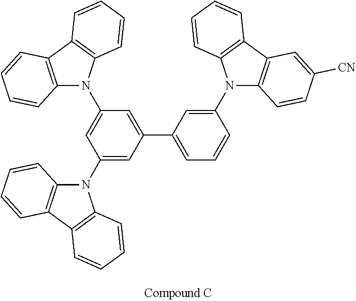 [SMILES: N#Cc1ccc2c(c1)c1ccccc1n2-c1cccc(-c2cc(-n3c4ccccc4c4ccccc43)cc(-n3c4ccccc4c4ccccc43)c2)c1]

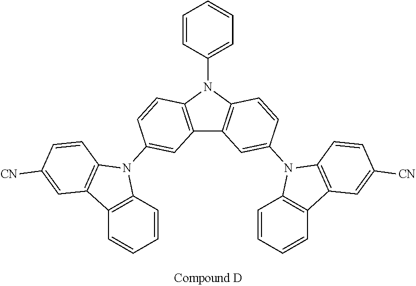

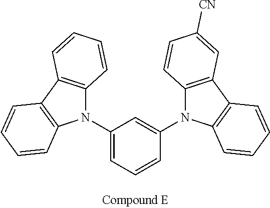 [SMILES: N#Cc1ccc2c(c1)c1ccccc1n2-c1cccc(-n2c3ccccc3c3ccccc32)c1]

From Table 5 and FIGS. 3 to 5, it may be seen that the organic light-emitting devices of Examples 1 to 3 have a lower driving voltage, a higher current density, a higher efficiency, a higher power, a higher luminance, a high quantum luminescent efficiency, and a longer lifespan than the organic light-emitting device of Comparative Examples 1 to 5.

Although not limited to a particular theory, in the case of Comparative Examples 3 and 4 respectively including Compounds C and D, it is understood that due to low T₁ energy levels of Compounds C and D, energy transition to a dopant in an emission layer is not smoothly carried out. Accordingly, it may be seen that the organic light-emitting devices of Comparative Examples 3 and 4 have lower quantum efficiency than the organic light-emitting devices of Examples 1 to 3.

The condensed cyclic compound according to embodiments has excellent electric characteristics and thermal stability. Accordingly, an organic light-emitting device using the condensed cyclic compound may have low driving voltage, high efficiency, high luminance, and a long lifespan.

It should be understood that exemplary embodiments described herein should be considered in a descriptive sense only and not for purposes of limitation. Descriptions of features or aspects within each exemplary embodiment should typically be considered as available for other similar features or aspects in other exemplary embodiments.

While one or more exemplary embodiments have been described with reference to the figures, it will be understood by those of ordinary skill in the art that various changes in form and details may be made therein without departing from the spirit and scope as defined by the following claims.

What is claimed is:

1. A condensed cyclic compound represented by Formula 1: 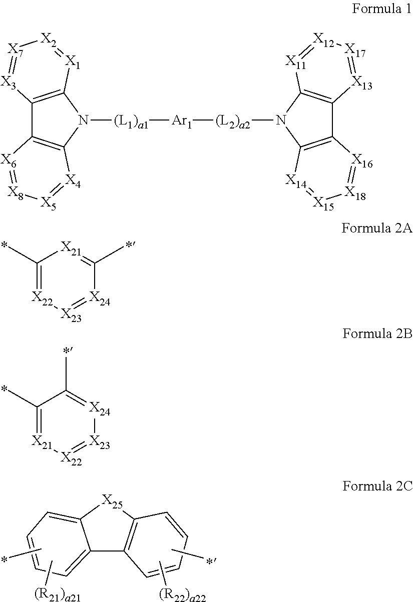
wherein in Formulae 1 and 2A to 2C,
X₁ is N or C(R₁), X₂ is N or C(R₂), X₃ is N or C(R₃), X₄ is N or C(R₄), X₅ is N or C(R₅), X₆ is N or C(R₆), X₇ is N or C(R₇), X₈ is N or C(R₈), X₁₁ is N or C(R₁₁), X₁₂ is N or C(R₁₂), X₁₃ is N or C(R₁₃), X₁₄ is N or C(R₁₄), X₁₅ is N or C(R₁₅), X₁₆ is N or C(R₁₆), X₁₇ is N or C(R₁₇), and X₁₈ is N or C(R₁₈),
R₁ to R₈ and R₁₁ to R₁₈ are each independently selected from a hydrogen, a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₇-C₆₀ arylalkyl group, a substituted or unsubstituted C₁-C₆₀ heteroaryl group, a substituted or unsubstituted C₂-C₆₀ heteroaryloxy group, a substituted or unsubstituted C₂-C₆₀ heteroarylthio group, a substituted or unsubstituted C₃-C₆₀ heteroarylalkyl group, a substituted or unsubstituted monovalent non-aromatic condensed polycyclic group, a substituted or unsubstituted monovalent non-aromatic condensed heteropolycyclic group (provided that the substituted or unsubstituted monovalent non-aromatic condensed heteropolycyclic group is not a substituted or unsubstituted carbazolyl group), and —Si(Q₁)(Q₂)(Q₃),
at least one selected from X₇, X₈, X₁₇, and X₁₈ is C(CN),
Ar₁ is represented by one of Formulae 2A to 2C,
X₂₁ is N or C(R₂₁), X₂₂ is N or C(R₂₂), X₂₃ is N or C(R₂₃), X₂₄ is N or C(R₂₄), and X₂₅ is O, S, P(═O)(R₂₅), Se, or Si(R₂₅)(R₂₆),
R₂₁ to R₂₆ are each independently selected from a hydrogen, a deuterium, C₁-C₄ alkyl group, a phenyl group, a pyridinyl group, a pyrimidinyl group, a pyrazinyl group, a pyridazinyl group, a triazinyl group, and —Si(Q₁₁)(Q₁₂)(Q₁₃),
a21 and a22 are each independently an integer selected from 0 to 3, and when a21 is 2 or greater, two or more groups R₂₁ may be identical to or different from each other, and when a22 is 2 or greater, two or more groups R₂₂ may be identical to or different from each other;
L₁ and L₂ are each independently selected from
a phenylene group, a pyridinylene group, a pyrimidinylene group, a pyrazinylene group, a pyridazinylene group, a triazinylene group, a dibenzofuranylene group, and a dibenzothiophenylene group; and
a phenylene group, a pyridinylene group, a pyrimidinylene group, a pyrazinylene group, a pyridazinylene group, a triazinylene group, a dibenzofuranylene group, and a dibenzothiophenylene group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, a phenyl group, a naphthyl group, a pyridinyl group, a pyrimidinyl group, a pyrazinyl group, a pyridazinyl group, a triazinyl group, and —Si(Q₂₁)(Q₂₂)(Q₂₃),
a1 and a2 are each independently an integer selected from 0 to 5, and when a1 is 2 or greater, two or more groups L₁ may be identical to or different from each other, and when a2 is 2 or greater, two or more groups L₂ may be identical to or different from each other;
each of * and *′ indicates a binding site to a neighboring atom;
at least one of substituents of the substituted C₁-C₆₀ alkyl group, substituted C₂-C₆₀ alkenyl group, substituted C₂-C₆₀ alkynyl group, substituted C₁-C₆₀ alkoxy group, substituted C₃-C₁₀ cycloalkyl group, substituted C₁-C₁₀ heterocycloalkyl group, substituted C₃-C₁₀ cycloalkenyl group, substituted C₁-C₁₀ heterocycloalkenyl group, substituted C₆-C₆₀ aryl group, substituted C₆-C₆₀ aryloxy group, substituted C₆-C₆₀ arylthio group, substituted C₇-C₆₀ arylalkyl group, substituted C₁-C₆₀ heteroaryl group, substituted C₂-C₆₀ heteroaryloxy group, substituted C₂-C₆₀ heteroarylthio group, substituted C₃-C₆₀ heteroarylalkyl group, substituted monovalent non-aromatic condensed polycyclic group, and substituted monovalent non-aromatic condensed heteropolycyclic group is selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, a monovalent non-aromatic condensed heteropolycyclic group (provided that the monovalent non-aromatic condensed heteropolycyclic group is not a carbazolyl group), and —Si(Q₃₁)(Q₃₂)(Q₃₃),
wherein Q₁ to Q₃, Q₁₁ to Q₁₃, Q₂₁ to Q₂₃, and Q₃₁ to Q₃₃ are each independently selected from a hydrogen, a C₁-C₆₀ alkyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group (provided that the monovalent non-aromatic condensed heteropolycyclic group is not a carbazolyl group),
provided that the condensed cyclic compound is not

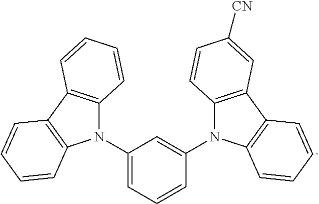 [SMILES: N#Cc1ccc2c(c1)c1ccccc1n2-c1cccc(-n2c3ccccc3c3ccccc32)c1]

2. The condensed cyclic compound of claim 1, wherein
R₁ to R₈ and R₁₁ to R₁₈ are each independently selected from
a hydrogen, a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, and a C₁-C₂₀ alkoxy group;
a C₁-C₂₀ alkyl group and a C₁-C₂₀ alkoxy group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a phenyl group, a naphthyl group, a pyridinyl group, a pyrimidinyl group, a pyrazinyl group, a pyridazinyl group, and a triazinyl group;
a cyclopentyl group, a cyclohexyl group, a cyclopentenyl group, a cyclohexenyl group, a cycloheptenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a phenalenyl group, a phenanthrenyl group, an anthracenyl group, a fluoranthrenyl group, a triphenylenyl group, a pyrenyl group, a chrysenyl group, a naphthacenyl group, a picenyl group, a perylenyl group, a pentaphenyl group, a hexacenyl group, a pyrrolyl group, an imidazolyl group, a pyrazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzoxazolyl group, a benzoimidazolyl group, a furanyl group, a benzofuranyl group, a thiophenyl group, a benzothiophenyl group, a thiazolyl group, an isothiazolyl group, a benzothiazolyl group, an isoxazolyl group, an oxazolyl group, a triazolyl group, a tetrazolyl group, an oxadiazolyl group, a triazinyl group, a dibenzofuranyl group, a dibenzothiophenyl group, an imidazopyrimidinyl group, and an imidazopyridinyl group;
a cyclopentyl group, a cyclohexyl group, a cyclopentenyl group, a cyclohexenyl group, a cycloheptenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a phenalenyl group, a phenanthrenyl group, an anthracenyl group, a fluoranthrenyl group, a triphenylenyl group, a pyrenyl group, a chrysenyl group, a naphthacenyl group, a picenyl group, a perylenyl group, a pentaphenyl group, a hexacenyl group, a pyrrolyl group, an imidazolyl group, a pyrazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzoxazolyl group, a benzoimidazolyl group, a furanyl group, a benzofuranyl group, a thiophenyl group, a benzothiophenyl group, a thiazolyl group, an isothiazolyl group, a benzothiazolyl group, an isoxazolyl group, an oxazolyl group, a triazolyl group, a tetrazolyl group, an oxadiazolyl group, a triazinyl group, a dibenzofuranyl group, a dibenzothiophenyl group, an imidazopyrimidinyl group, and an imidazopyridinyl group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₂-C₂₀ alkenyl group, a C₂-C₂₀ alkynyl group, a C₁-C₂₀ alkoxy group, a phenyl group, a naphthyl group, an anthracenyl group, a pyrenyl group, a phenanthrenyl group, a fluorenyl group, a pyridinyl group, a pyrimidinyl group, a pyrazinyl group, a pyridazinyl group, a triazinyl group, a quinolinyl group, an isoquinolinyl group, a phthalazinyl group, a quinoxalinyl group, a cinnolinyl group, a quinazolinyl group, and —Si(Q₃₁)(Q₃₂)(Q₃₃); and
—Si(Q₁)(Q₂)(Q₃),
wherein Q₁ to Q₃ and Q₃₁ to Q₃₃ are each independently selected from a hydrogen, a C₁-C₂₀ alkyl group, a C₁-C₂₀ alkoxy group, a phenyl group, a naphthyl group, an anthracenyl group, a pyrenyl group, a phenanthrenyl group, a fluorenyl group, a pyridinyl group, a pyrimidinyl group, a pyrazinyl group, a pyridazinyl group, a triazinyl group, a quinolinyl group, an isoquinolinyl group a phthalazinyl group, a quinoxalinyl group, a cinnolinyl group, and a quinazolinyl group.

3. The condensed cyclic compound of claim 1, wherein
R₁ to R₆ and R₁₁ to R₁₆ are each independently selected from
a hydrogen, a deuterium, —F, —Cl, —Br, —I, a hydroxyl group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, and a C₁-C₁₀ alkoxy group;
a phenyl group, a naphthyl group, a pyridinyl group, a pyrimidinyl group, a pyrazinyl group, a pyridazinyl group, and a triazinyl group;
a phenyl group, a naphthyl group, a pyridinyl group, a pyrimidinyl group, a pyrazinyl group, a pyridazinyl group, and a triazinyl group, each substituted with at least one selected from a deuterium, —F, —Cl, —Br, —I, a hydroxyl group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, a phenyl group, a naphthyl group, a pyridinyl group, a pyrimidinyl group, a pyrazinyl group, a pyridazinyl group, a triazinyl group, and —Si(Q₃₁)(Q₃₂)(Q₃₃); and
—Si(Q₁)(Q₂)(Q₃),
R₇, R₈, R₁₇, and R₁₈ are each independently selected from a hydrogen, a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, and a C₁-C₁₀ alkoxy group;
a phenyl group, a naphthyl group, a pyridinyl group, a pyrimidinyl group, a pyrazinyl group, a pyridazinyl group, and a triazinyl group;
a phenyl group, a naphthyl group, a pyridinyl group, a pyrimidinyl group, a pyrazinyl group, a pyridazinyl group, and a triazinyl group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, a phenyl group, a naphthyl group, a pyridinyl group, a pyrimidinyl group, a pyrazinyl group, a pyridazinyl group, a triazinyl group, and —Si(Q₃₁)(Q₃₂)(Q₃₃); and
—Si(Q₁)(Q₂)(Q₃),
wherein Q₁ to Q₃ and Q₃₁ to Q₃₃ are each independently selected from a hydrogen, a C₁-C₁₀ alkyl group, a C₁-C₁₀ alkoxy group, a phenyl group, a naphthyl group, a pyridinyl group, a pyrimidinyl group, a pyrazinyl group, a pyridazinyl group, and a triazinyl group.

4. The condensed cyclic compound of claim 1, wherein
R₁ to R₈ and R₁₁ to R₁₈ are each independently selected from a hydrogen, a deuterium, a cyano group, a methyl group, an ethyl group, an n-propyl group, an isopropyl group, an n-butyl group, an isobutyl group, a sec-butyl group, a tert-butyl group, an n-pentyl group, an isopentyl group, a sec-pentyl group, a tert-pentyl group, an n-hexyl group, an iso-hexyl group, a sec-hexyl group, a tert-hexyl group, and —Si(Q₁)(Q₂)(Q₃), wherein Q₁ to Q₃ are each independently selected from a hydrogen, a C₁-C₁₀ alkyl group, a C₁-C₁₀ alkoxy group, and a phenyl group.

5. The condensed cyclic compound of claim 1, wherein
each of R₁ to R₆ and R₁₁ to R₁₆ is not a cyano group.

6. The condensed cyclic compound of claim 1, wherein
Ar₁ is represented by one of Formulae 2A-1 to 2A-10, 2B-1 to 2B-8, and 2C-1 to 2C-9: 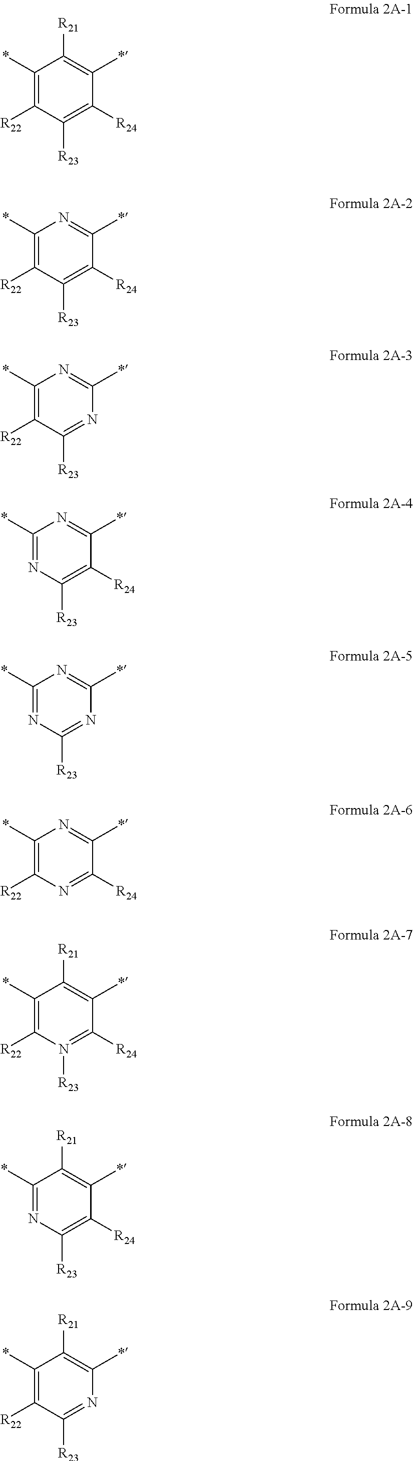 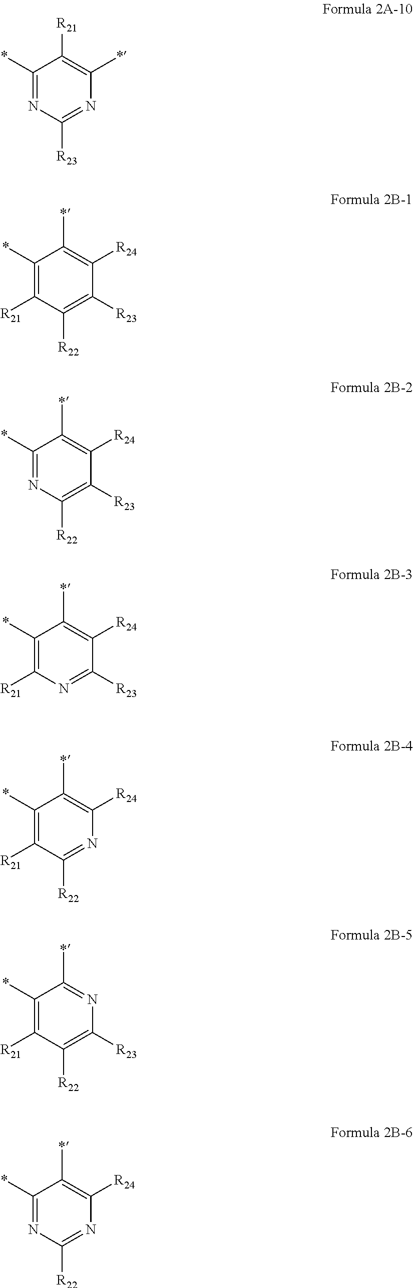 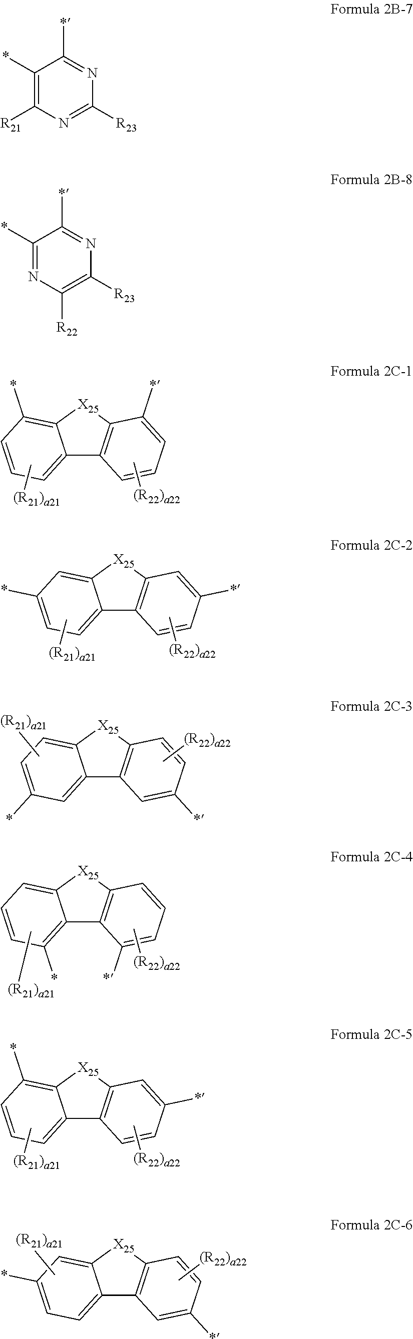 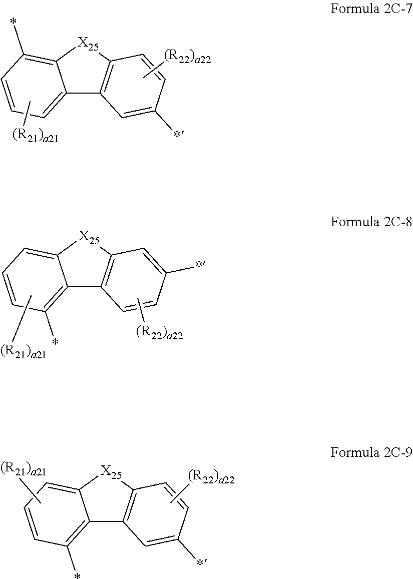
wherein in Formulae 2A-1 to 2A-10, 2B-1 to 2B-8, and 2C-1 to 2C-9,
X₂₅ is O, S, P(═O)(R₂₅), Se, or Si(R₂₅)(R₂₆),
R₂₁ to R₂₄ are each independently selected from a hydrogen, a deuterium, a methyl group, an ethyl group, an n-propyl group, an isopropyl group, an n-butyl group, an isobutyl group, a sec-butyl group, a tert-butyl group, a phenyl group, a pyridinyl group, a pyrimidinyl group, a pyrazinyl group, a pyridazinyl group, a triazinyl group, and —Si(Q₁₁)(Q₁₂)(Q₁₃),
R₂₅ and R₂₆ are each independently selected from a hydrogen, a deuterium, a methyl group, an ethyl group, an n-propyl group, an isopropyl group, an n-butyl group, an isobutyl group, a sec-butyl group, a tert-butyl group, a phenyl group, a pyridinyl group, a pyrimidinyl group, a pyrazinyl group, a pyridazinyl group and a triazinyl group,
wherein Q₁₁ to Q₁₃ are each independently selected from a hydrogen, a C₁-C₁₀ alkyl group, a C₁-C₁₀ alkoxy group, and a phenyl group,
a21 and a22 are each independently 0 or 1, and
each of * and *′ indicates a binding site to a neighboring atom.

7. The condensed cyclic compound of claim 1, wherein
L₁ and L₂ are each independently selected from
a phenylene group, a pyridinylene group, a pyrimidinylene group, and a triazinylene group; and
a phenylene group, a pyridinylene group, a pyrimidinylene group, and a triazinylene group, each substituted with at least one selected from a deuterium, a methyl group, an ethyl group, an n-propyl group, an isopropyl group, an n-butyl group, an isobutyl group, a sec-butyl group, a tert-butyl group, a phenyl group, a pyridinyl group, a pyrimidinyl group, a pyrazinyl group, a pyridazinyl group, a triazinyl group, and —Si(Q₂₁)(Q₂₂)(Q₂₃).

8. The condensed cyclic compound of claim 1, wherein
L₁ and L₂ are each independently selected from
a phenylene group; and
a phenylene group, substituted with at least one selected from a deuterium, a methyl group, an ethyl group, an n-propyl group, an isopropyl group, an n-butyl group, an isobutyl group, a sec-butyl group, a tert-butyl group, a phenyl group, a pyridinyl group, a pyrimidinyl group, a pyrazinyl group, a pyridazinyl group, a triazinyl group, and —Si(Q₂₁)(Q₂₂)(Q₂₃),
wherein Q₂₁ to Q₂₃ are each independently selected from a hydrogen, a C₁-C₁₀ alkyl group, a C₁-C₁₀ alkoxy group, and a phenyl group, and
a1 and a2 are each independently 0 or 1.

9. The condensed cyclic compound of claim 6, wherein
a1 and a2 in Formula 1 is 0 and Ar₁ is represented by one of Formulae 2A-1, 2A-2, 2A-10, 2B-1, 2B-8 and 2C-3.

10. The condensed cyclic compound of claim 1, wherein
regarding Formula 1, the sum of a1 and a2 is 1 or more, and *-(L₁)ₐ₁-Ar₁-(L₂)ₐ₂-*′ is represented by one of Formulae 3-1 to 3-57: 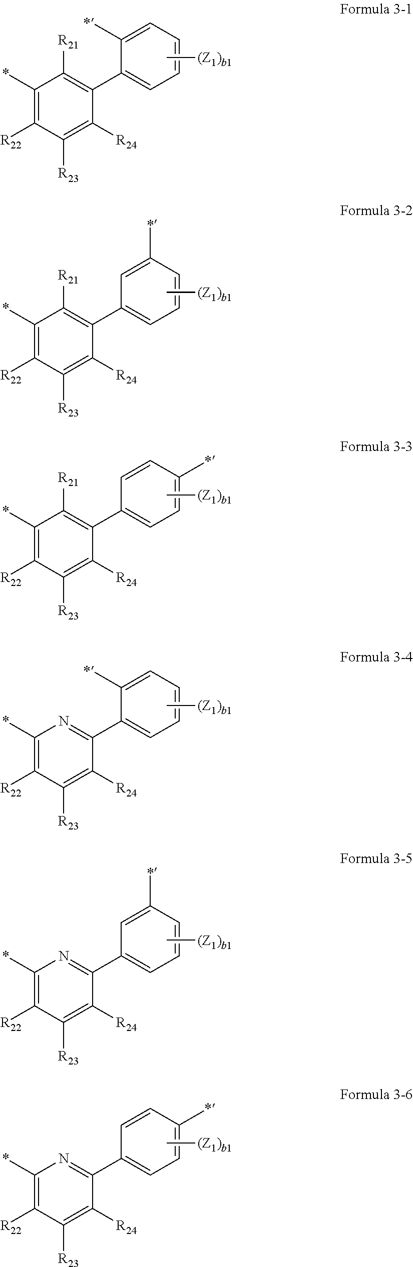 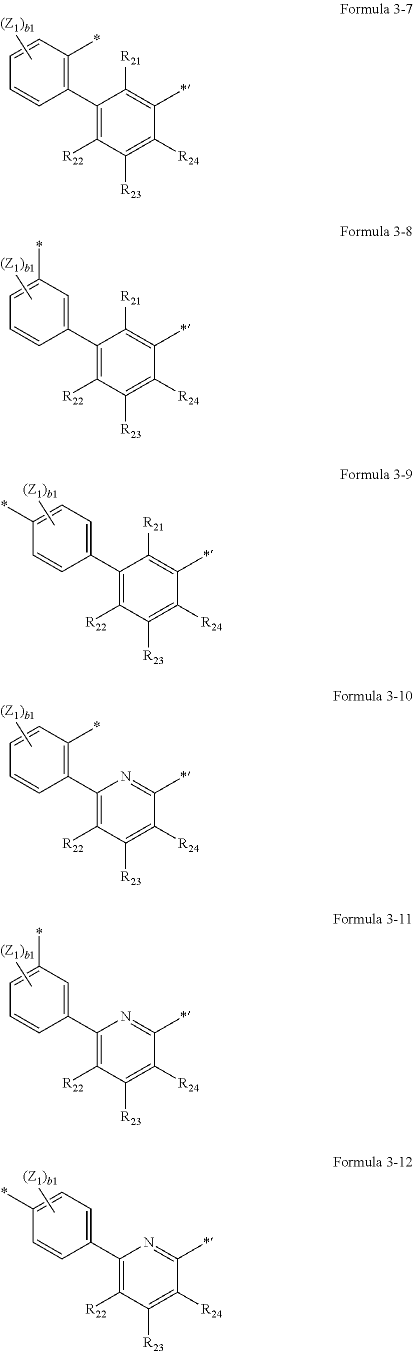 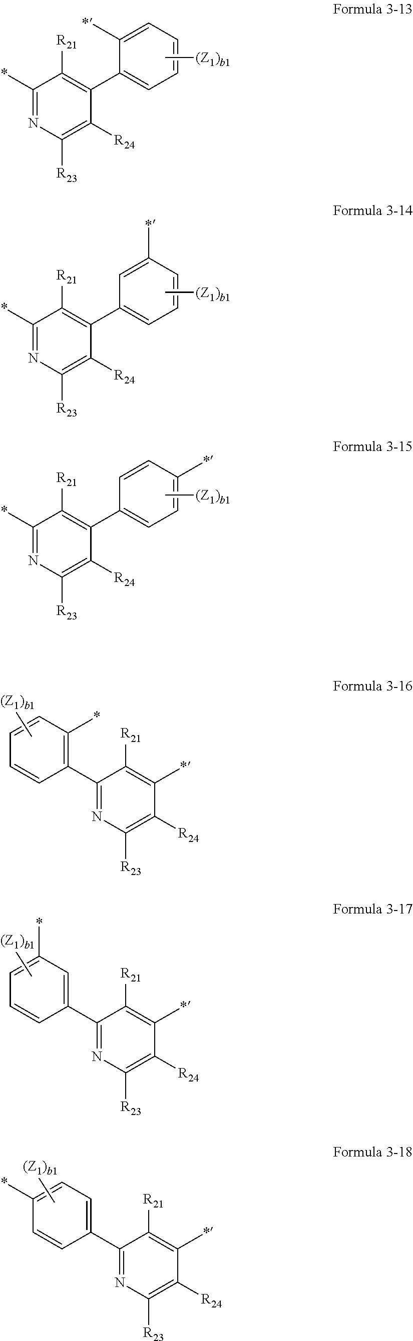 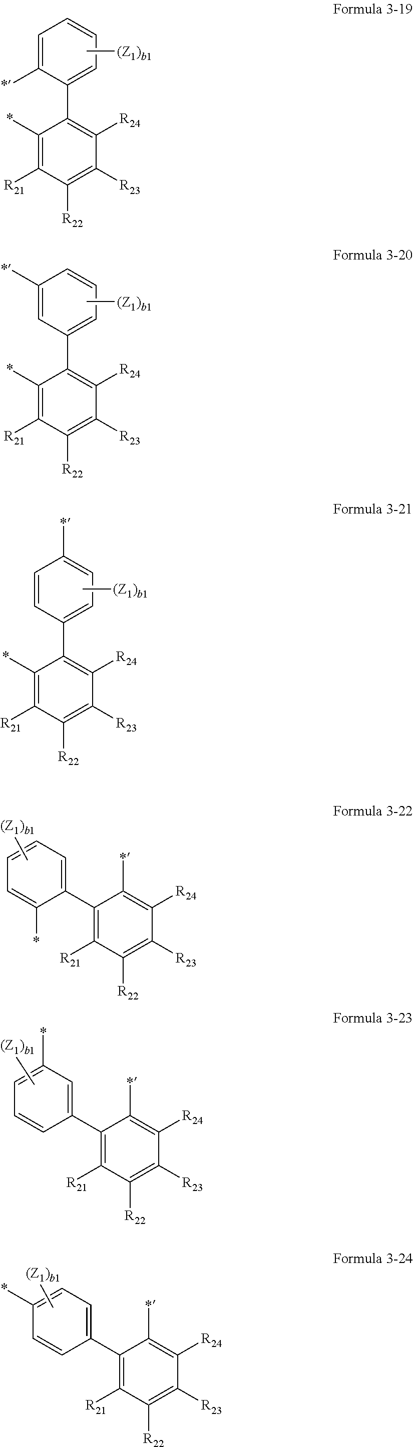 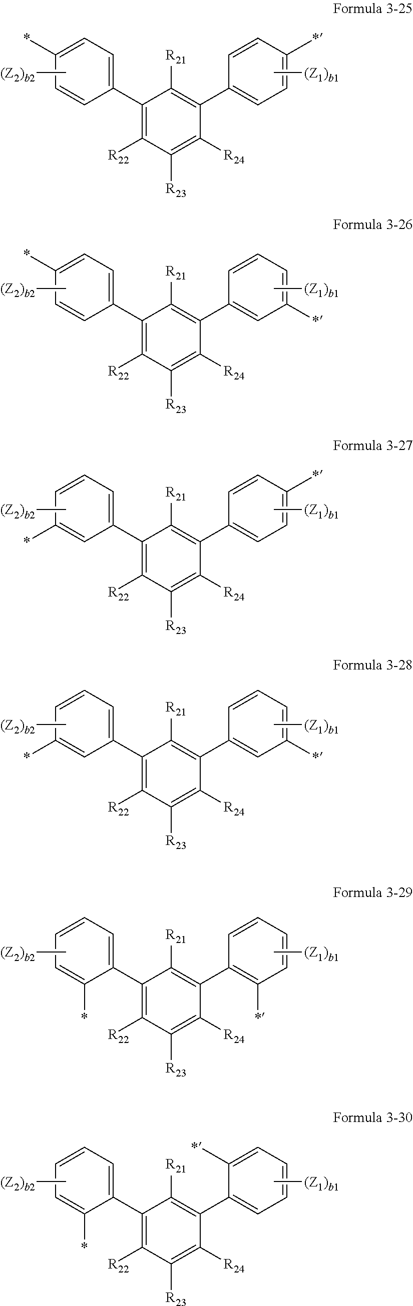 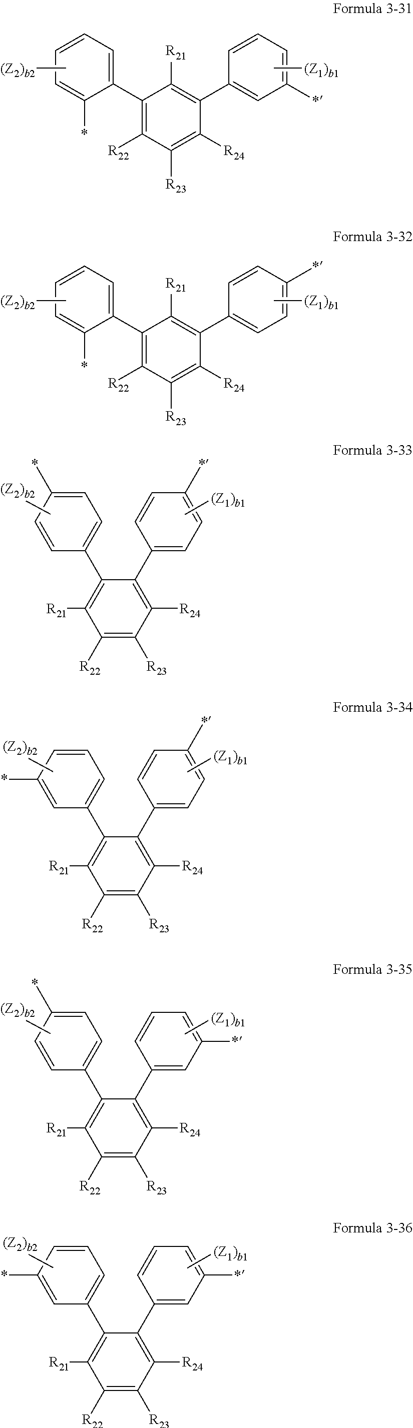 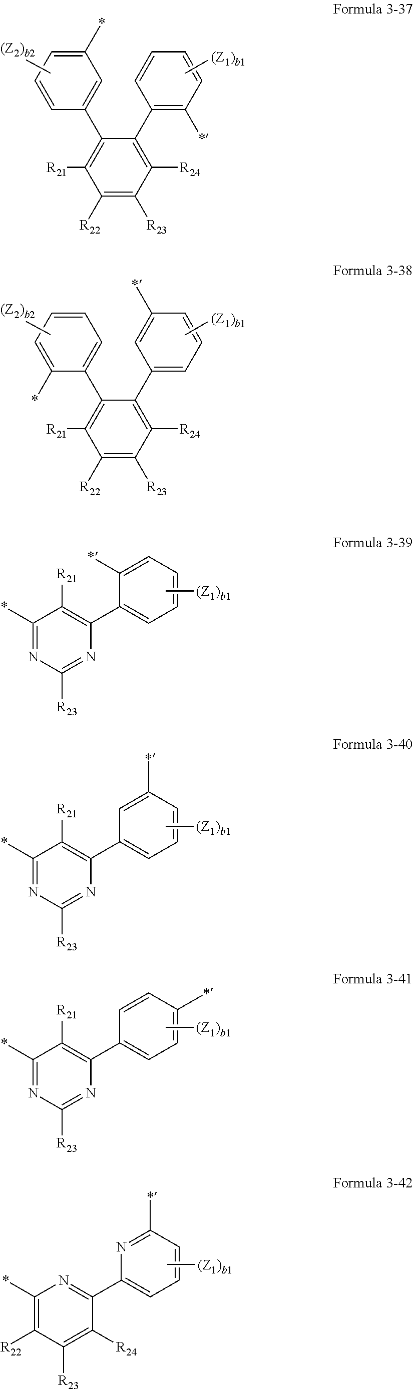 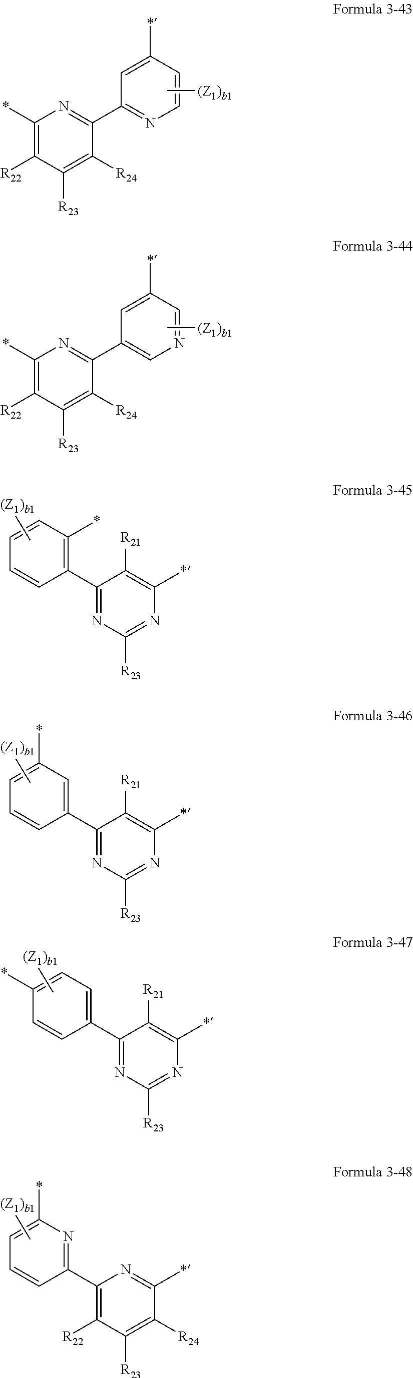 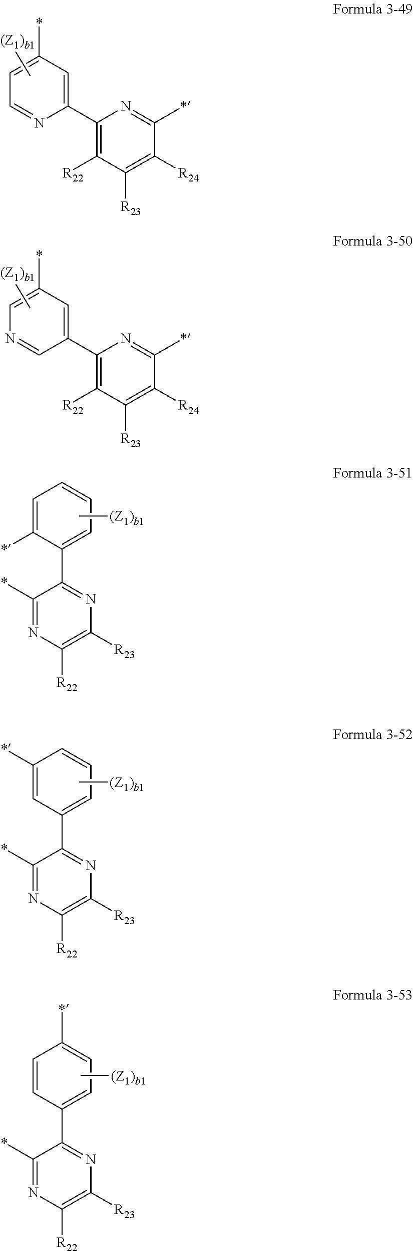 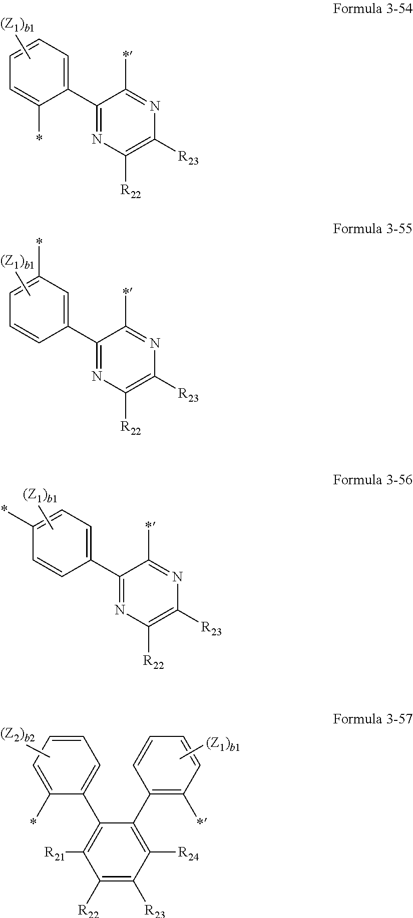
wherein in Formulae 3-1 to 3-57,
R₂₁ to R₂₄, Z₁, and Z₂ are each independently selected from a hydrogen, a deuterium, a methyl group, an ethyl group, an n-propyl group, an isopropyl group, an n-butyl group, an isobutyl group, a sec-butyl group, a tert-butyl group, a phenyl group, a pyridinyl group, a pyrimidinyl group, a pyrazinyl group, a pyridazinyl group, a triazinyl group, and —Si(Q₁₁)(Q₁₂)(Q₁₃),
wherein Q₁₁ to Q₁₃ are each independently selected from a hydrogen, a C₁-C₁₀ alkyl group, a C₁-C₁₀ alkoxy group, and a phenyl group,
b1 and b2 are each independently 0, 1, or 2, and
each of * and *′ indicates a binding site to a neighboring nitrogen atom.

11. The condensed cyclic compound of claim 1, wherein
the condensed cyclic compound is represented by one of Formulae 1A to 1E: 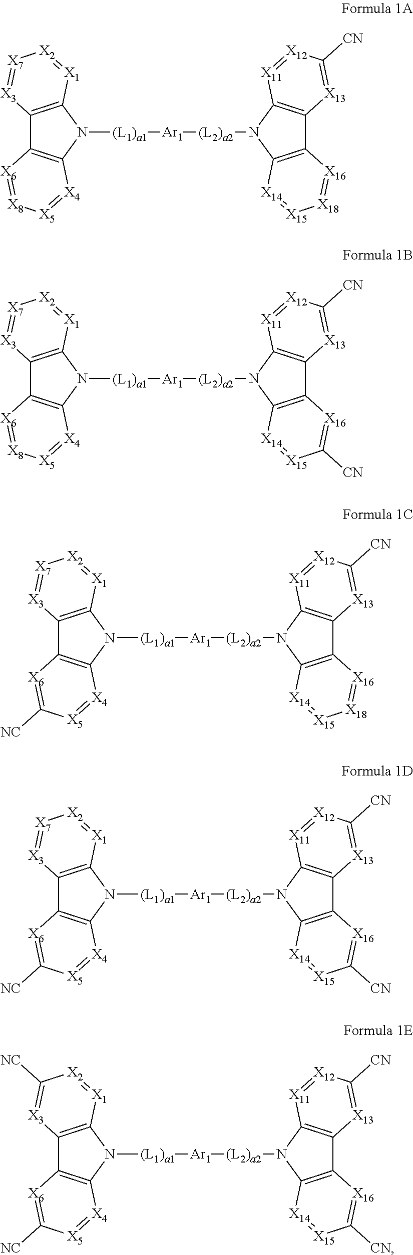
wherein X₁ to X₈, X₁₁ to X₁₈, Ar₁, L₁, L₂, a1, and a2 in Formulae 1A to 1E are the same as in claim 1.

12. The condensed cyclic compound of claim 1, wherein
the condensed cyclic compound is represented by one of Formulae 1(1) to 1(3): 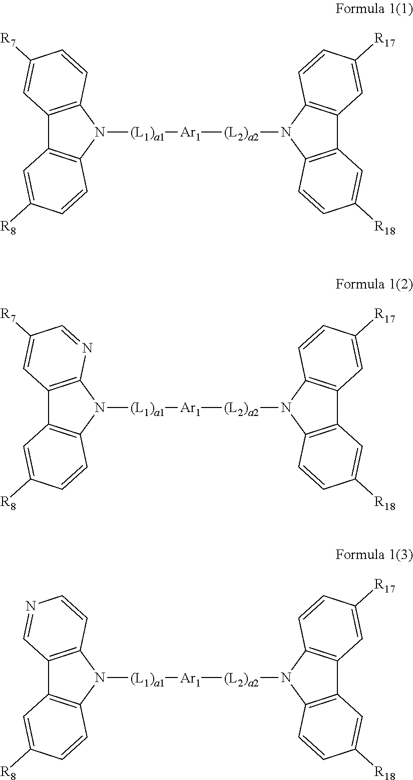
wherein in Formulae 1(1) to 1(3),
Ar₁, L₁, L₂, a1, and a2 are the same as in claim 1,
R₇, R₈, R₁₇, and R₁₈ are each independently selected from
a hydrogen, a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a methyl group, an ethyl group, an n-propyl group, an isopropyl group, an n-butyl group, an isobutyl group, a sec-butyl group, a tert-butyl group, an n-pentyl group, an isopentyl group, a sec-pentyl group, a tert-pentyl group, an n-hexyl group, an iso-hexyl group, a sec-hexyl group, a tert-hexyl group, an n-heptyl group, an iso-heptyl group, a sec-heptyl group, a tert-heptyl group, an n-octyl group, an iso-octyl group, a sec-octyl group, a tert-octyl group, an n-nonyl group, an iso-nonyl group, a sec-nonyl group, a tert-nonyl group, an n-decyl group, an isodecyl group, a sec-decyl group, a tert-decyl group, a methoxy group, an ethoxy group, a propoxy group, a butoxy group, a pentoxy group, a phenyl group, a naphthyl group, a pyridinyl group, a pyrimidinyl group, and a triazinyl group;
a methyl group, an ethyl group, an n-propyl group, an isopropyl group, an n-butyl group, an isobutyl group, a sec-butyl group, a tert-butyl group, an n-pentyl group, an isopentyl group, a sec-pentyl group, a tert-pentyl group, an n-hexyl group, an iso-hexyl group, a sec-hexyl group, a tert-hexyl group, an n-heptyl group, an iso-heptyl group, a sec-heptyl group, a tert-heptyl group, an n-octyl group, an iso-octyl group, a sec-octyl group, a tert-octyl group, an n-nonyl group, an iso-nonyl group, a sec-nonyl group, a tert-nonyl group, an n-decyl group, an isodecyl group, a sec-decyl group, a tert-decyl group, a methoxy group, an ethoxy group, a propoxy group, a butoxy group, a pentoxy group, a phenyl group, a naphthyl group, a pyridinyl group, a pyrimidinyl group, and a triazinyl group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, and a phosphoric acid group or a salt thereof; and
—Si(Q₁)(Q₂)(Q₃),
wherein Q₁ to Q₃ are each independently selected from a hydrogen, a C₁-C₁₀ alkyl group, a C₁-C₁₀ alkoxy group, and a phenyl group,
at least one selected from R₇, R₈, R₁₇, and R₁₈ in Formulae 1 (1) and 1(2) is a cyano group, and
at least one selected from R₈, R₁₇, and R₁₈ in Formula 1(3) is a cyano group.

13. The condensed cyclic compound of claim 1, wherein
the condensed cyclic compound is represented by one of Formulae 1A(1) to 1E(1), 1A(2) to 1E(2) and 1A(3) to 1 D(3): 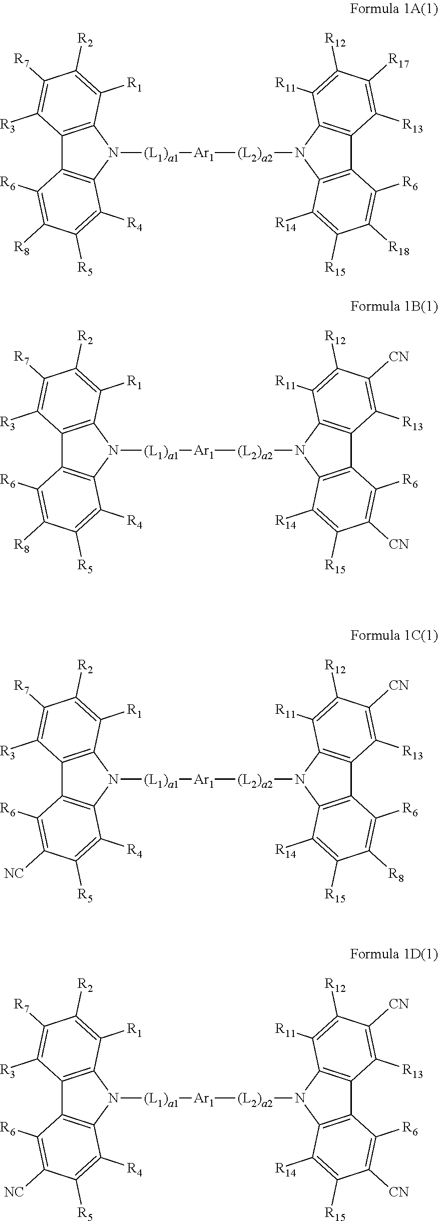 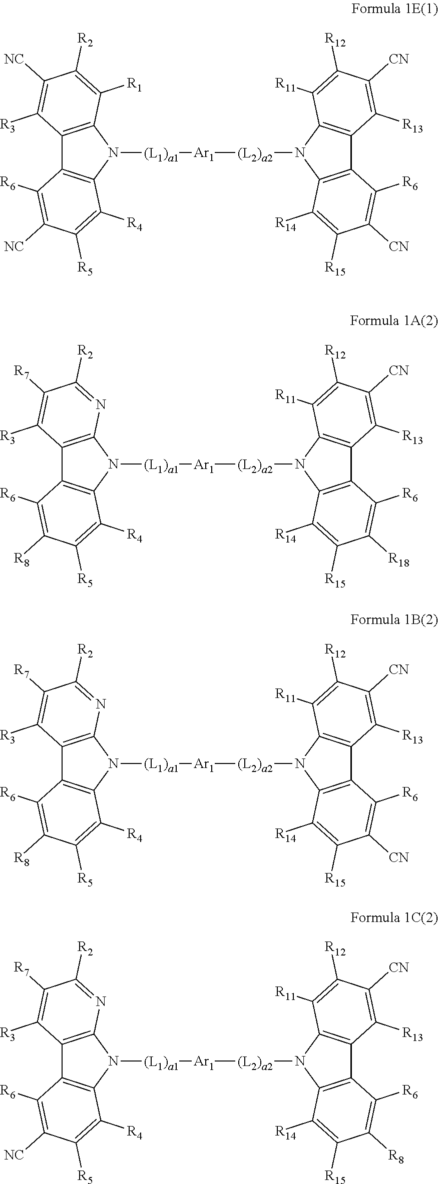 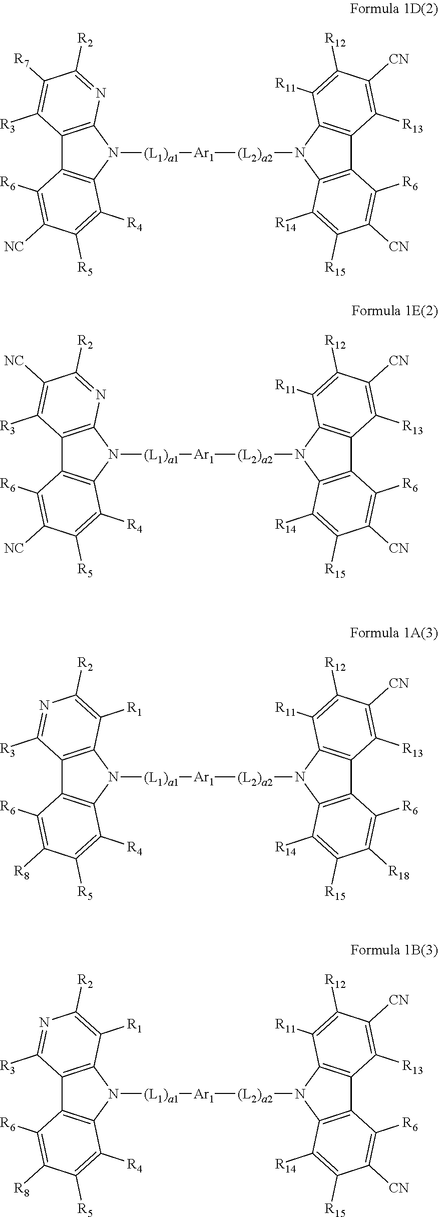 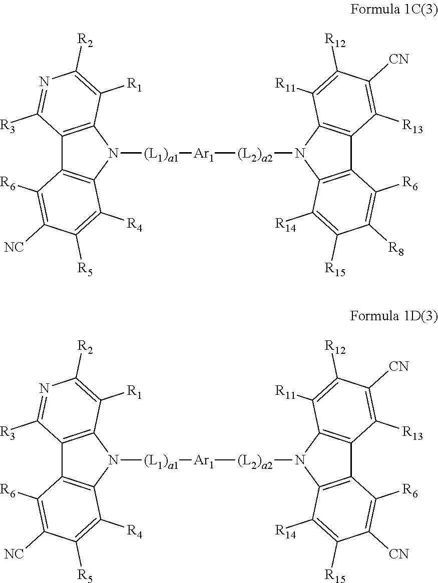
wherein in Formulae 1A(1) to 1E(1), 1A(2) to 1E(2) and 1A(3) to 1 D(3),
R₁ to R₈, R₁₁ to R₁₆, and R₁₈ are each independently selected from a hydrogen, a deuterium, —F, —Cl, —Br, —I, a hydroxyl group, a nitro group, an amino group, an amidino group, a hydrazine group, a hydrazone group, a carboxylic acid group or a salt thereof, a sulfonic acid group or a salt thereof, a phosphoric acid group or a salt thereof, a methyl group, an ethyl group, an n-propyl group, an isopropyl group, an n-butyl group, an isobutyl group, a sec-butyl group, a tert-butyl group, an n-pentyl group, an isopentyl group, a sec-pentyl group, a tert-pentyl group, an n-hexyl group, an iso-hexyl group, a sec-hexyl group, a tert-hexyl group, an n-heptyl group, an iso-heptyl group, a sec-heptyl group, a tert-heptyl group, an n-octyl group, an iso-octyl group, a sec-octyl group, a tert-octyl group, an n-nonyl group, an iso-nonyl group, a sec-nonyl group, a tert-nonyl group, an n-decyl group, an isodecyl group, a sec-decyl group, a tert-decyl group, a methoxy group, an ethoxy group, a propoxy group, a butoxy group, a pentoxy group, a phenyl group, a naphthyl group, a pyridinyl group, a pyrimidinyl group, a triazinyl group, and —Si(Q₁)(Q₂)(Q₃),
Ar₁ is represented by Formulae 2A-1, 2A-2, 2A-10, 2B-1, 2B-8 or 2C-3,
L₁ and L₂ are each independently selected from
a phenylene group, a pyridinylene group, a pyrimidinylene group, a pyrazinylene group, a pyridazinylene group, a triazinylene group, a dibenzofuranylene group, and a dibenzothiophenylene group; and
a phenylene group, a pyridinylene group, a pyrimidinylene group, a pyrazinylene group, a pyridazinylene group, a triazinylene group, a dibenzofuranylene group, and a dibenzothiophenylene group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, a phenyl group, a naphthyl group, a pyridinyl group, a pyrimidinyl group, a pyrazinyl group, a pyridazinyl group, a triazinyl group and —Si(Q₂₁)(Q₂₂)(Q₂₃),
a1 and a2 are each independently 0 or 1,
wherein Q₁ to Q₃, and Q₂₁ to Q₂₃ are each independently selected from a hydrogen, a C₁-C₁₀ alkyl group, a C₁-C₁₀ alkoxy group, and a phenyl group: 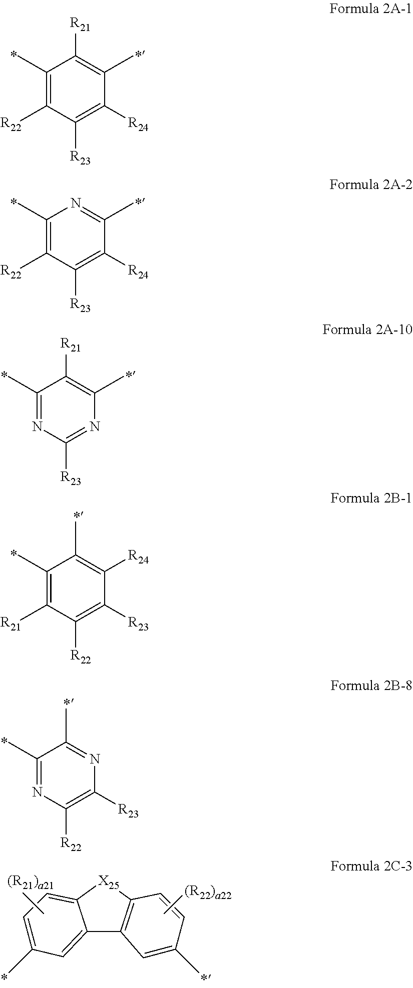
wherein in Formulae 2A-1, 2A-2, 2A-10, 2B-1, 2B-8 and 2C-3,
X₂₅ is O, S, P(═O)(R₂₅), Se, or Si(R₂₅)(R₂₆),
R₂₁ to R₂₄ are each independently selected from a hydrogen, a deuterium, a methyl group, an ethyl group, an n-propyl group, an isopropyl group, an n-butyl group, an isobutyl group, a sec-butyl group, a tert-butyl group, a phenyl group, a pyridinyl group, a pyrimidinyl group, a pyrazinyl group, a pyridazinyl group, a triazinyl group, and —Si(Q₁₁)(Q₁₂)(Q₁₃),
R₂₅ and R₂₆ are each independently selected from a hydrogen, a deuterium, a methyl group, an ethyl group, an n-propyl group, an isopropyl group, an n-butyl group, an isobutyl group, a sec-butyl group, a tert-butyl group, a phenyl group, a pyridinyl group, a pyrimidinyl group, a pyrazinyl group, a pyridazinyl group and a triazinyl group, a21 and a22 are each independently 0 or 1,
wherein Q₁₁ to Q₁₃ are each independently selected from a hydrogen, a C₁-C₁₀ alkyl group, a C₁-C₁₀ alkoxy group, and a phenyl group.

14. The condensed cyclic compound of claim 1, wherein
the condensed cyclic compound is one of Compounds 1 to 56:

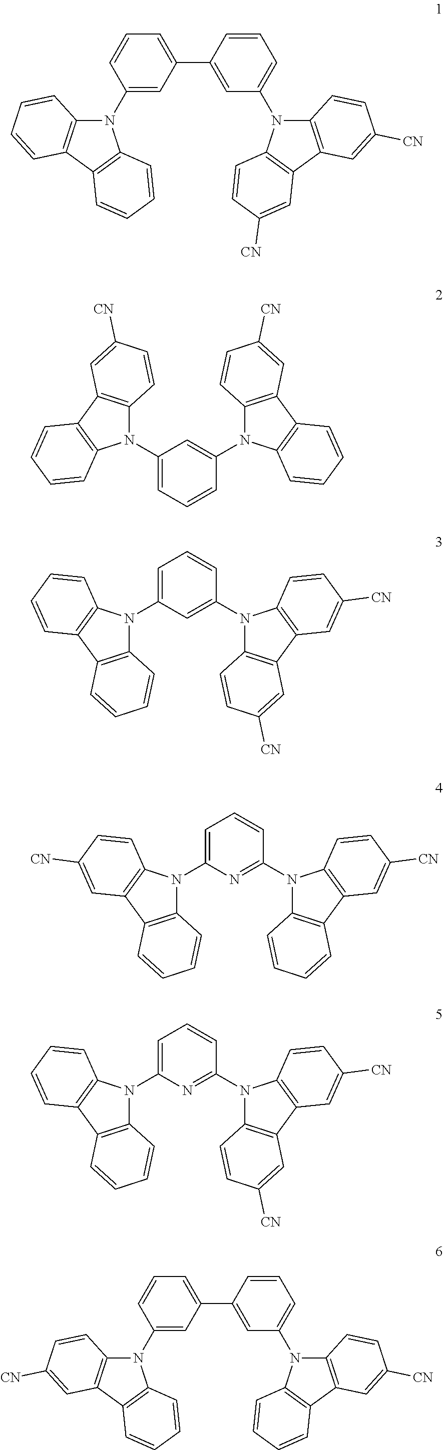

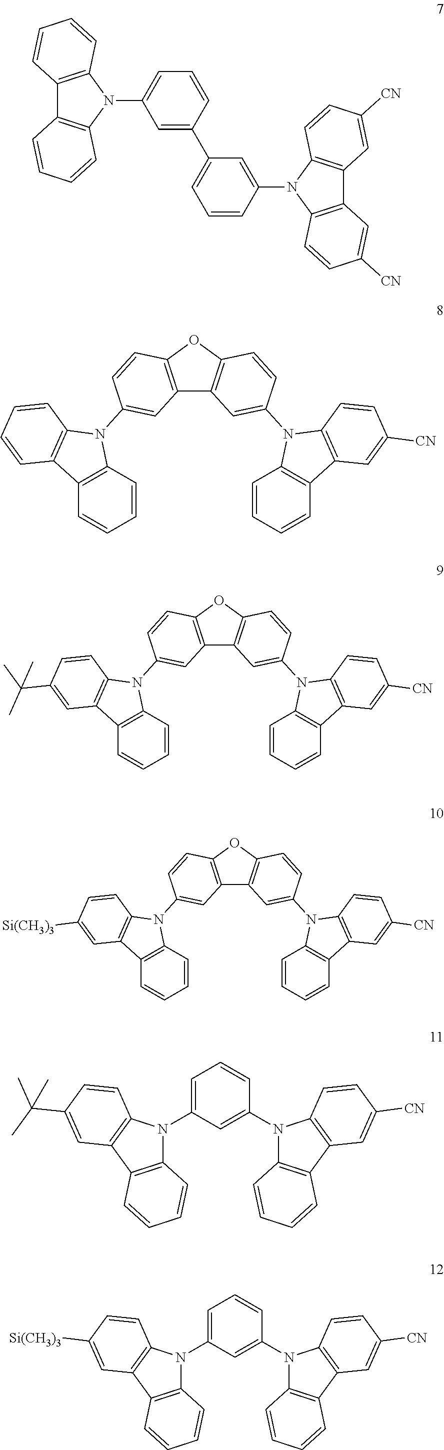

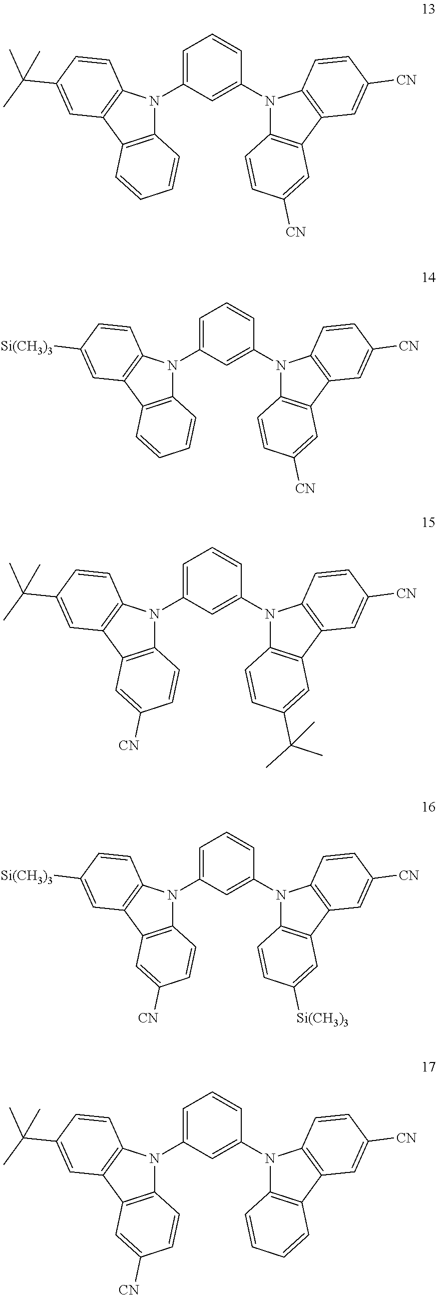

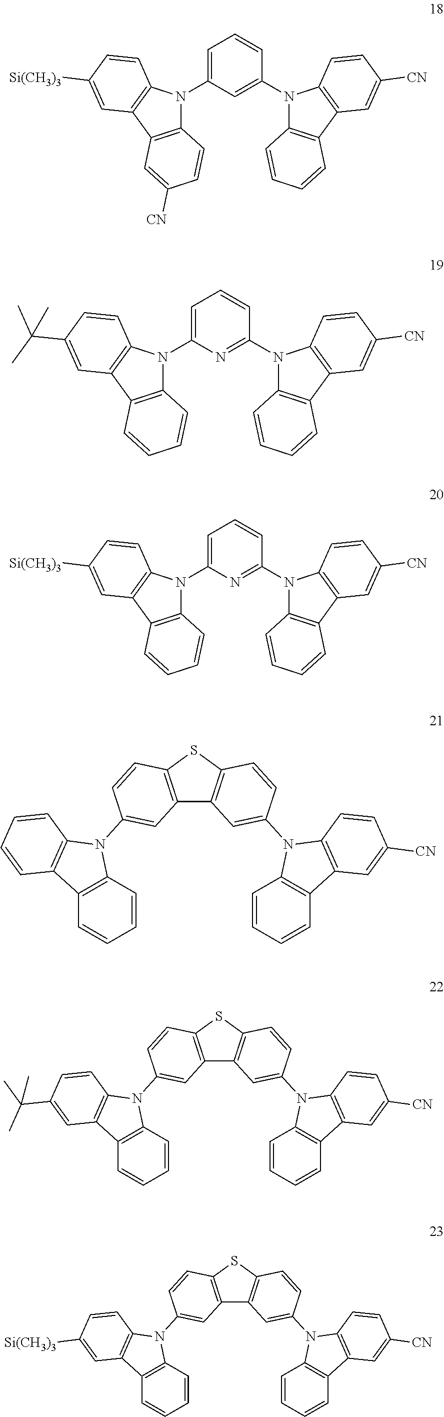

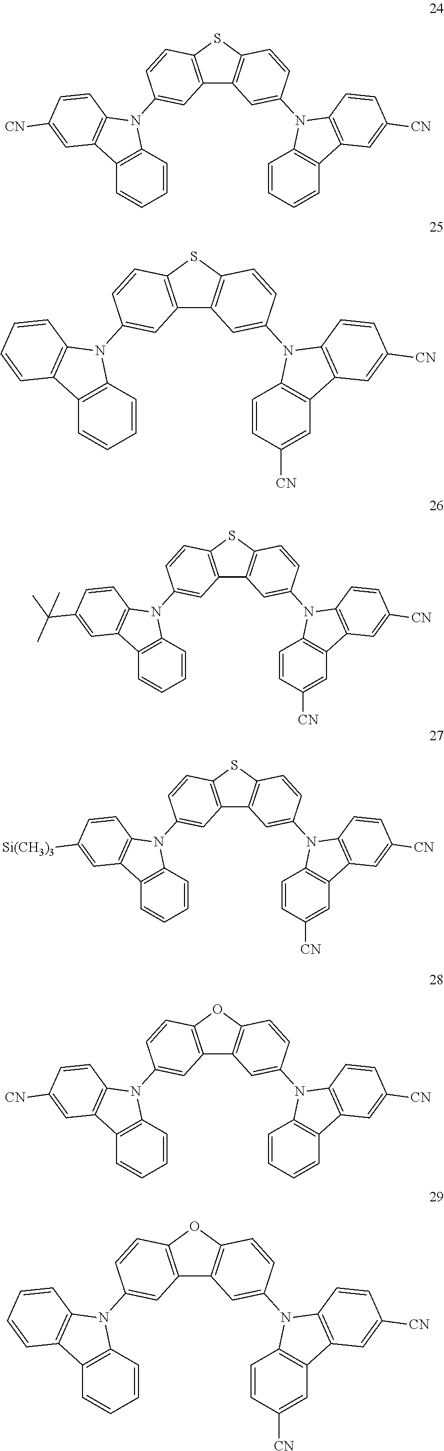

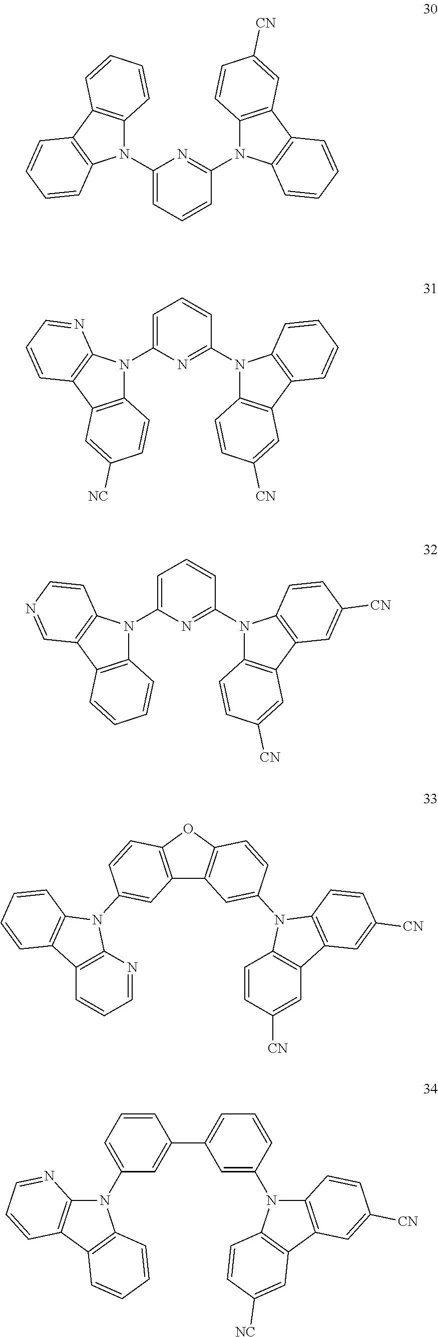 [SMILES: N#Cc1ccc2c(c1)c1ccccc1n2-c1cccc(-n2c3ccccc3c3ccccc32)n1 ; N#Cc1ccc2c(c1)c1ccccc1n2-c1cccc(-n2c3ccc(C#N)cc3c3cccnc32)n1 ; N#Cc1ccc2c(c1)c1cc(C#N)ccc1n2-c1cccc(-n2c3ccccc3c3cnccc32)n1 ; N#Cc1ccc2c(c1)c1cc(C#N)ccc1n2-c1ccc2oc3ccc(-n4c5ccccc5c5cccnc54)cc3c2c1 ; N#Cc1ccc2c(c1)c1cc(C#N)ccc1n2-c1cccc(-c2cccc(-n3c4ccccc4c4cccnc43)c2)c1]

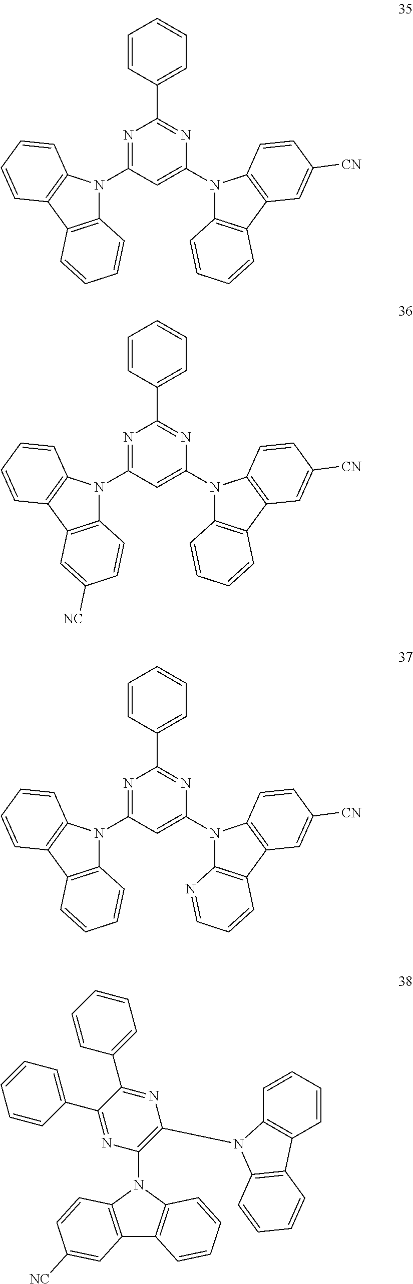 [SMILES: N#Cc1ccc2c(c1)c1ccccc1n2-c1cc(-n2c3ccccc3c3ccccc32)nc(-c2ccccc2)n1 ; N#Cc1ccc2c(c1)c1ccccc1n2-c1cc(-n2c3ccccc3c3cc(C#N)ccc32)nc(-c2ccccc2)n1 ; N#Cc1ccc2c(c1)c1cccnc1n2-c1cc(-n2c3ccccc3c3ccccc32)nc(-c2ccccc2)n1 ; N#Cc1ccc2c(c1)c1ccccc1n2-c1nc(-c2ccccc2)c(-c2ccccc2)nc1-n1c2ccccc2c2ccccc21]

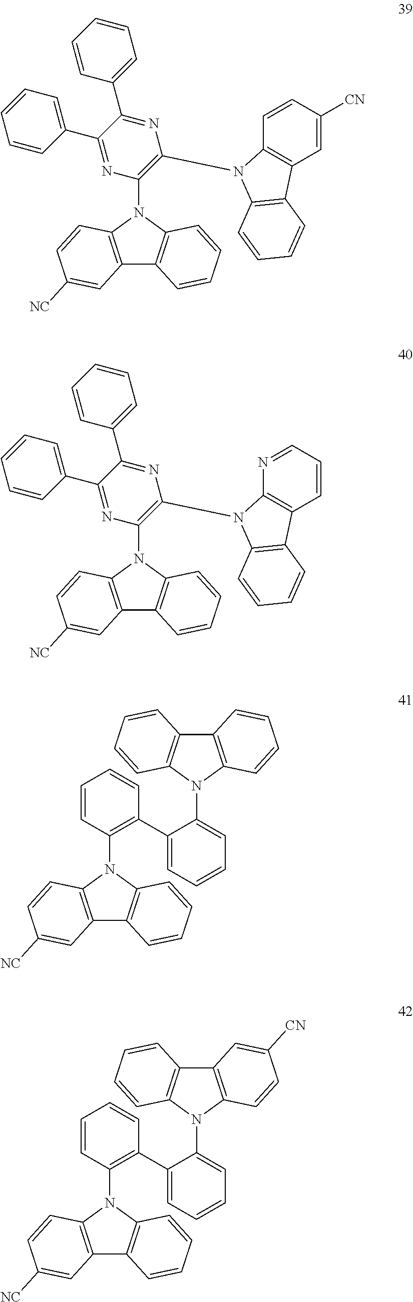 [SMILES: N#Cc1ccc2c(c1)c1ccccc1n2-c1nc(-c2ccccc2)c(-c2ccccc2)nc1-n1c2ccccc2c2cc(C#N)ccc21 ; N#Cc1ccc2c(c1)c1ccccc1n2-c1nc(-c2ccccc2)c(-c2ccccc2)nc1-n1c2ccccc2c2cccnc21 ; N#Cc1ccc2c(c1)c1ccccc1n2-c1ccccc1-c1ccccc1-n1c2ccccc2c2ccccc21 ; N#Cc1ccc2c(c1)c1ccccc1n2-c1ccccc1-c1ccccc1-n1c2ccccc2c2cc(C#N)ccc21]

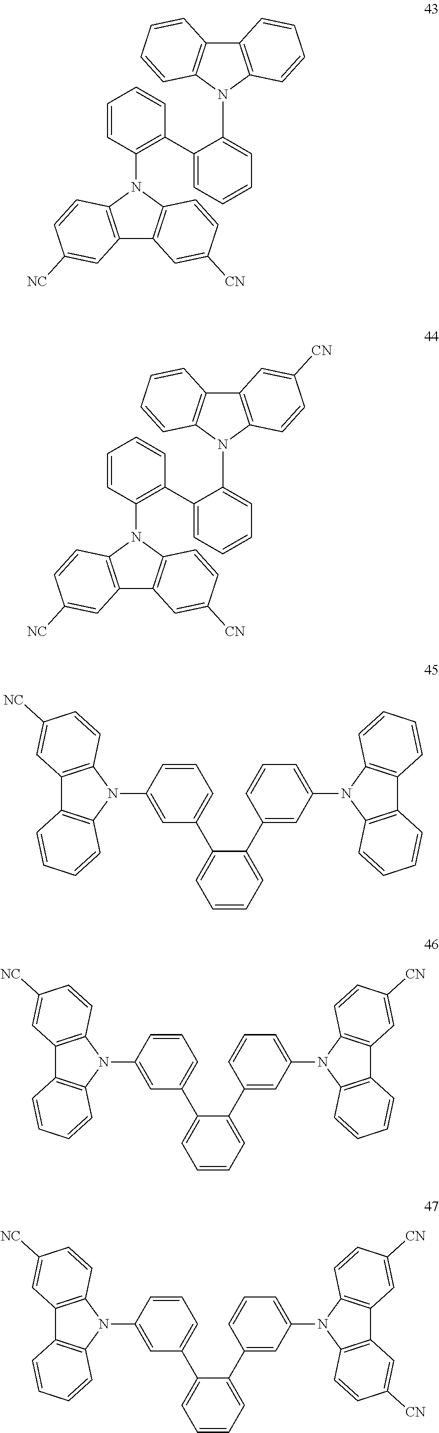 [SMILES: N#Cc1ccc2c(c1)c1cc(C#N)ccc1n2-c1ccccc1-c1ccccc1-n1c2ccccc2c2ccccc21 ; N#Cc1ccc2c(c1)c1ccccc1n2-c1ccccc1-c1ccccc1-n1c2ccc(C#N)cc2c2cc(C#N)ccc21 ; N#Cc1ccc2c(c1)c1ccccc1n2-c1cccc(-c2ccccc2-c2cccc(-n3c4ccccc4c4ccccc43)c2)c1 ; N#Cc1ccc2c(c1)c1ccccc1n2-c1cccc(-c2ccccc2-c2cccc(-n3c4ccccc4c4cc(C#N)ccc43)c2)c1 ; N#Cc1ccc2c(c1)c1ccccc1n2-c1cccc(-c2ccccc2-c2cccc(-n3c4ccc(C#N)cc4c4cc(C#N)ccc43)c2)c1]

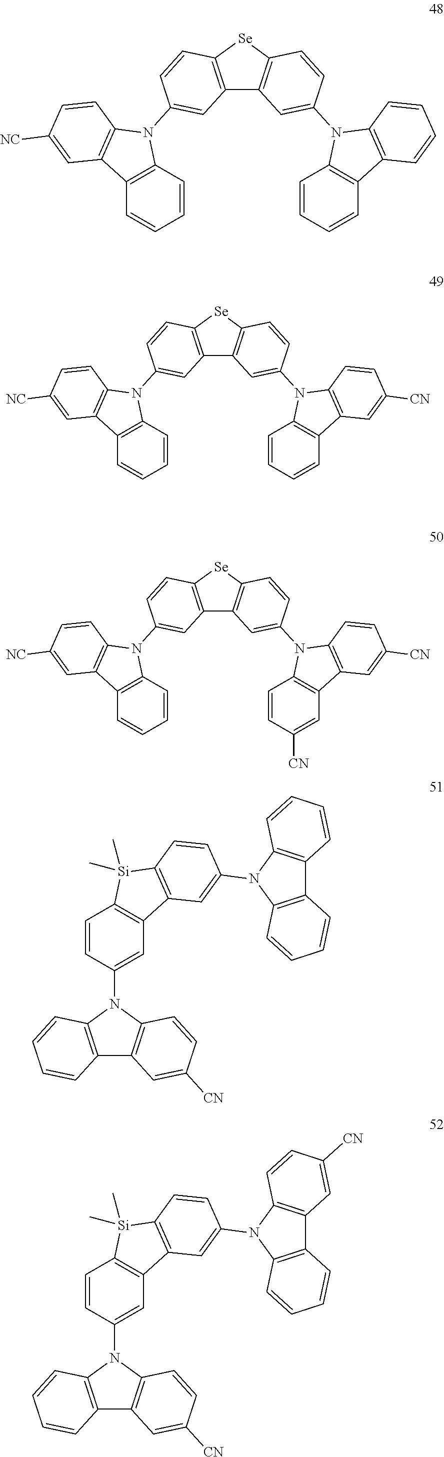

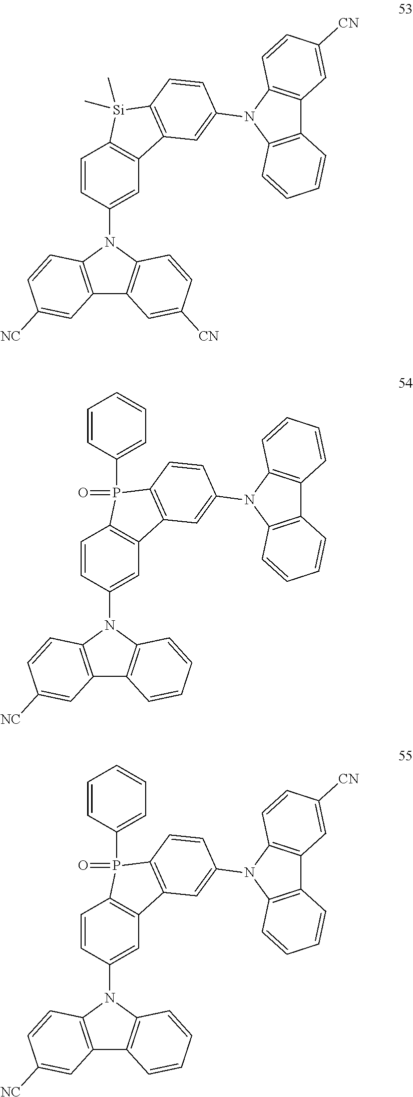 [SMILES: C[Si]1(C)c2ccc(-n3c4ccccc4c4cc(C#N)ccc43)cc2-c2cc(-n3c4ccc(C#N)cc4c4cc(C#N)ccc43)ccc21 ; N#Cc1ccc2c(c1)c1ccccc1n2-c1ccc2c(c1)-c1cc(-n3c4ccccc4c4ccccc43)ccc1P2(=O)c1ccccc1 ; N#Cc1ccc2c(c1)c1ccccc1n2-c1ccc2c(c1)-c1cc(-n3c4ccccc4c4cc(C#N)ccc43)ccc1P2(=O)c1ccccc1]

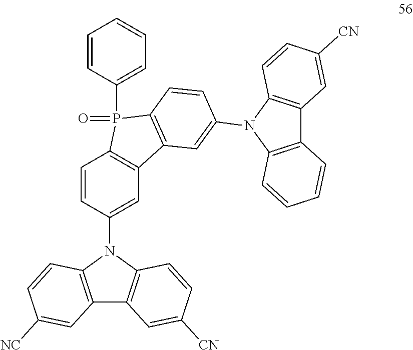 [SMILES: N#Cc1ccc2c(c1)c1ccccc1n2-c1ccc2c(c1)-c1cc(-n3c4ccc(C#N)cc4c4cc(C#N)ccc43)ccc1P2(=O)c1ccccc1]

15. An organic light-emitting device comprising:
a first electrode;
a second electrode; and
an organic layer disposed between the first electrode and the second electrode,
wherein the organic layer comprises an emission layer and at least one of the condensed cyclic compounds represented by Formula 1 of claim 1.

16. The organic light-emitting device of claim 15, wherein
the first electrode is an anode,
the second electrode is a cathode, and
the organic layer comprises:
i) a hole transport region disposed between the first electrode and the emission layer, wherein the hole transport region comprises at least one selected from a hole injection layer, a hole transport layer, and an electron blocking layer; and
ii) an electron transport region disposed between the emission layer and the second electrode, wherein the electron transport region comprises at least one selected from a hole blocking layer, an electron transport layer, and an electron injection layer.

17. The organic light-emitting device of claim 15, wherein
the emission layer comprises the condensed cyclic compound represented by Formula 1.

18. The organic light-emitting device of claim 15, wherein the emission layer comprises the condensed cyclic compound represented by Formula 1 and a phosphorescent dopant, and wherein an amount of the condensed cyclic compound is greater than an amount of the phosphorescent dopant.

19. The organic light-emitting device of claim 17, wherein
the emission layer emits blue light.

20. The organic light-emitting device of claim 15, wherein
the emission layer comprises the condensed cyclic compound represented by Formula 1, and wherein the condensed cyclic compound represented by Formula 1 is a thermally activated delayed fluorescence emitter.
